(19) United States
(12) Patent Application Publication
(10) Pub. No.: US 2023/0357446 A1
(43) Pub. Date: Nov. 9, 2023

(54) COMPOSITIONS AND METHODS FOR UNIVERSAL TUMOR CELL KILLING

(71) Applicant: Regeneron Pharmaceuticals, Inc., Tarrytown, NY (US)

(72) Inventors: Eric Smith, New York, NY (US); Dimitris Skokos, New York, NY (US); Kara Olson, White Plains, NY (US); Lauric Haber, Rye Brook, NY (US); Joyce Wei, New York, NY (US); Chia-yang Lin, Scardale, NY (US)

(21) Appl. No.: 18/133,288

(22) Filed: Apr. 11, 2023

Related U.S. Application Data
(60) Provisional application No. 63/347,330, filed on May 31, 2022.
(60) Provisional application No. 63/329,762, filed on Apr. 11, 2022.

(51) Int. Cl. C07K 16/46; A61P 35/00

(52) U.S. Cl. CPC C07K 16/468; A61P 35/00; C07K 2317/31

(57) ABSTRACT

Provided herein are methods of mediating killing of a tumor cell by administering to the subject a multispecific antigen binding molecule with a first antigen binding region specific for first target antigen and a second antigen binding region specific for a CD28 protein. In some embodiments, the tumor cell does not express the target antigen. In some embodiments, the tumor cell is not predicted to express the target antigen.

### CROSS REFERENCE TO RELATED APPLICATIONS

This application claims the benefit of U.S. Provisional Application No. 63/329,762, filed Apr. 11, 2022, and U.S. Provisional Application No. 63/347,330, filed May 31, 2022, the entire contents of each are hereby incorporated by reference.

### BACKGROUND

Cancer is one of North America's leading causes of death. Despite decades of research, many cancers continue to not respond or develop resistance to traditional chemotherapies and other therapeutics. Recently, advances in immunotherapy have produced some promising therapies, but even these therapeutics tend to prove effective only in specific patients and cancer types. Thus, there is a need for novel treatments for cancer.

### SUMMARY

The methods and compositions provided herein are based, in part, on the unexpected discovery that CD28 bispecific antibodies targeting tumor associated antigens, antigens targeting cells in the tumor microenvironment, or an immune antigen (e.g., an antigen expressed on the surface of an immune cell in the tumor or tumor microenvironment) can induce killing of cancer or tumor cells lacking expression of such antigens.

In certain aspects, provided herein are methods and compositions for mediating killing of a tumor cell in a tumor in a subject by administering to the subject a multispecific (e.g., bispecific) antigen binding molecule with a first antigen binding region specific for a target antigen and a second antigen binding region specific for a CD28 protein, wherein the tumor cell does not express or is not predicted to express the target antigen. In some embodiments, at least a subset of tumor cells in the tumor do not express the target antigen.

In some aspects, provided herein are methods and compositions for inducing killing of tumor cells and/or inducing T cell activation against tumor cells in a tumor in a subject, the method comprising administering to the subject a multispecific antigen binding molecule with a first antigen binding region specific for a target antigen and a second antigen binding region specific for a CD28 protein. In some embodiments, at least a subset of tumor cells in the tumor do not express the target antigen.

In some aspects, also provided herein are methods and compositions for treating cancer in a subject with a tumor, the method comprising administering to the subject a multispecific antigen binding molecule with a first antigen binding region specific for a target antigen and a second antigen binding region specific for a CD28 protein. In some embodiments, at least a subset of tumor cells in the tumor do not express the target antigen.

In some embodiments, the target antigen is a tumor associated antigen (TAA). In some embodiments, the target antigen is an antigen associated with a tumor microenvironment (e.g., the microenvironment of the tumor in the subject). For example, in some embodiments the target antigen is an antigen on an immune cell, on tumor cell stroma, or on the extracellular matrix within the tumor microenvironment. Examples of extracellular matrix antigens include nectin (e.g., nectin-3 or nectin-4), versican (VACN), fibronectin and carcinoembryonic antigen-related cell adhesion molecules (CEACAM) protein antigens.

In some embodiments, the methods provided herein may further comprise determining that at least the subset of the tumor cells in the tumor do not express the target antigen. In some embodiments, the tumor cells do not express the target antigen if the expression of the target antigen is below the level of detection or below signal to noise ratio.

In some aspects, provided herein are methods of treating cancer in a subject, comprising i) determining that the subject comprises a tumor comprising tumor cells that do not express target antigen, and ii) administering to the subject a multispecific antigen binding molecule comprising a first antigen binding region specific for the target antigen and a second antigen binding region specific for a CD28 protein.

In some aspects, provided herein are methods of selecting a subject for cancer therapy, the method comprising: i) determining that the subject comprises a tumor comprising tumor cells that do not express a target antigen; and ii) administering to the subject a multispecific antigen binding molecule with a first antigen binding region specific for the target antigen and a second antigen binding region specific for a CD28 protein, optionally wherein the tumor cells do not express the target antigen if the expression of the target antigen is below the level of detection or below signal to noise ratio, thereby selecting a subject for cancer therapy.

In some embodiments, the target antigen is a tumor associated antigen (TAA). In some embodiments, the tumor may be a heterogeneous tumor further comprising tumor cells that express the TAA. In some embodiments, the tumor microenvironment of the tumor comprises cells that express the TAA. In some embodiments, at least 20%, at least 30%, at least 40%, at least 50%, at least 60%, at least 70%, or at least 80% of the tumor cells in the tumor do not express the TAA. In some embodiments, at least 10%-100%, 20%-100%, 30%-100%, 40%-100%, 50%-100%, 60%-100%, 70%-100%, 80%-100% or 90%-100% of the tumor cells in the tumor do not express the TAA.

In some embodiments, the target antigen is an antigen associated with the tumor microenvironment of the tumor, such as an antigen associated with the tumor stroma, an antigen associated with the extracellular matrix of the tumor, an antigen associated with a blood vessel in the tumor microenvironment, or an antigen associated with a cancer-associated fibroblast

In some embodiments, the target antigen is an antigen associated with the tumor stroma selected from PSA, CEA, CA-125, CA-19, COL10, FAP, B7H3, LRRC15, and fibronectin-EDB isoform.

In some embodiments, the target antigen is an antigen associated with the extracellular matrix of the tumor selected from nectin (e.g., nectin-3 or nectin-4), versican (VACN), fibronectin and a carcinoembryonic antigen-related cell adhesion molecules (CEACAM) protein.

In some embodiments, the tumor microenvironment of the tumor comprises cells that express the target antigen.

In some embodiments, the target antigen is an antigen expressed on the surface of a cancer-associated fibroblast (e.g., α-smooth muscle actin (α-SMA), fibroblast activation protein (FAP), S100A4, platelet-derived growth factor receptors (PDGFRα/β), vimentin, PDPN, CD70, CD10, GPR77, CD10, CD74, CD146, CAV1, Saa3-, or CD49e).

In some embodiments, the target antigen is an antigen expressed on the surface of a blood vessel in the tumor microenvironment, such as DLK1, EphA2, HBB, NG2, NRP1, NRP2, PDGFRβ, PSMA, RGS5, TEM1, VEGFR1 or VEGFR2.

In some embodiments, the target antigen is an immune antigen. In some embodiments, the immune antigen is an antigen expressed on the surface of an immune cell. The immune cell may be a macrophage, a neutrophil, an eosinophil, a basophil, a mast cell, a monocyte, a dendritic cell, a natural killer cell, a T cell or a B cell. In some embodiments, the immune cell has infiltrated the tumor or tumor microenvironment of the tumor. The immune antigen may be selected from any one of the immune antigens listed in Table 4.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the tumor cells in the tumor do not express the target antigen. In some embodiments, at least 10%-100%, 20%-100%, 30%-100%, 40%-100%, 50%-100%, 60%-100%, 70%-100%, 80%-100% or 90%-100% of the tumor cells in the tumor do not express the target antigen.

In some embodiments, the tumor comprises immune cells (e.g., B cells and/or CD20-expressing cells). In some embodiments, the tumor cells that do not express the target antigen also do not express CD20. The target antigen may be selected from CD38, EGFR, CD22, MUC16, PSMA, CA9, FOLR1, HER2, and SLAMF7. The target antigen may be CD22.

In some embodiments, the multispecific antigen binding molecule may be a bispecific antibody (e.g., any one of the bispecific antibodies listed in Table 3) or a bispecific antibody fragment such as a bispecific T-engaging antibody (BiTE), a dual-affinity re-targeting molecule (DART), and a tandem diabody (TandAb). In some embodiments, the multispecific antigen binding molecule is administered to the subject conjointly with a second multispecific antigen binding molecule with a first antigen binding region specific for a second target antigen (e.g., a second tumor associated antigen (TAA2)) and a second antigen binding region specific for a CD3 protein. In some embodiments, the second multispecific antigen binding molecule is a bispecific antibody or a bispecific antibody fragment, such as a bispecific T-engaging antibody (BiTE), a dual-affinity re-targeting molecule (DART), and a tandem diabody (TandAb).

The second target antigen may be selected from any one of the antigens listed in Table 2. The second target antigen may be a CD20 protein. In some embodiments, the second multispecific antigen binding molecule is selected from any one of the multispecific antigen binding molecules in Table 5. The multispecific antigen binding molecule may demonstrate a costimulatory effect when administered conjointly with the second multispecific antigen binding molecule. In some embodiments, the costimulatory effect is one or more of the following: activating T-cells, inducing IL-2 release, inducing CD25+ up-regulation in PBMCs, and increasing T-cell mediated cytotoxicity. In some embodiments, the tumor cells are tumor cells of a B cell cancer. B cell cancers include, but are not limited to, diffuse large B-cell lymphoma (DLBCL), follicular lymphoma, chronic lymphocytic leukemia (CLL), small lymphocytic lymphoma (SLL), mantle cell lymphoma (MCL), marginal zone lymphomas, burkitt lymphoma, lymphoplasmacytic lymphoma (Waldenstrom macroglobulinemia), hairy cell leukemia, primary central nervous system (CNS) lymphoma, or primary intraocular lymphoma (lymphoma of the eye).

In some embodiments, the tumor is a solid tumor. The tumor may be an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngreal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

The multispecific antigen binding molecule may be administered systemically, intravenously, subcutaneously, or intramuscularly. The multispecific antigen binding molecule may be administered to the subject in a pharmaceutically acceptable formulation. In some embodiments, the method further comprises administering an additional anti-cancer agent. The additional anti-cancer agent may be a chemotherapeutic agent, an immune checkpoint inhibitor, CAR-T cells, or a tumor vaccine. The immune checkpoint inhibitor may be an anti-PD-1 antibody, an anti-PDL1 antibody, an anti-CTLA4 antibody, or an anti-LAG3 antibody.

In some embodiments, the subject is afflicted with a refractory cancer.

In some aspects, provided herein are methods of treating cancer in a subject with a tumor, inducing or mediating killing of tumor cells in a tumor in a subject, and/or inducing T cell activation against tumor cells in a tumor in a subject, the method comprising administering to the subject: a first multispecific antigen binding molecule with an antigen binding region specific for a first target antigen) and an antigen binding region specific for a CD28 protein; and a second multispecific antigen binding molecule with an antigen binding region specific for a second target antigen and an antigen binding region specific for a CD3 protein, wherein the first target antigen is not the same antigen as the second target antigen. In some embodiments, the first and/or second target antigens are tumor associated antigens (TAAs).

In some embodiments, the method further comprises determining that the tumor comprises a subset of tumor cells that do not express the first target antigen.

In some aspects, provided herein are methods of treating cancer in a subject with a tumor, inducing or mediating killing of tumor cells in the tumor in a subject, and/or inducing T cell activation against tumor cells in a tumor in a subject, the method comprising determining if the tumor comprises tumor cells that do not express a first target antigen, administering to the subject: a first multispecific antigen binding molecule with an antigen binding region specific for the first target antigen and an antigen binding region specific for a CD28 protein; and a second multispecific antigen binding molecule with an antigen binding region specific for a second target antigen and an antigen binding region specific for a CD3 protein, wherein the first target antigen is not the same antigen as the second target antigen. In some embodiments, the first and/or second target antigens are tumor associated antigens (TAAs).

In some embodiments, at least a subset of the tumor cells in the tumor do not express the first target antigen. In some embodiments, at least 20%, at least 30%, at least 40%, at least 50%, at least 60%, at least 70%, or at least 80% of the tumor cells in the tumor do not express the first target antigen. In some embodiments, at least a subset of the tumor cells in the tumor do not express the second target antigen. In some embodiments, the tumor is a heterogeneous tumor comprising cells that express the first target antigen and cells that express the second target antigen. In some embodiments, the tumor cells in the tumor do not express both the first target antigen and the second target antigen.

In some embodiments, the first target antigen is an antigen associated with the tumor microenvironment of the tumor, such as an antigen associated with the tumor stroma, an antigen associated with the extracellular matrix of the tumor, an antigen associated with a blood vessel in the tumor microenvironment, or an antigen associated with a cancer-associated fibroblast

In some embodiments, the first target antigen is an antigen associated with the tumor stroma selected from PSA, CEA, CA-125, CA-19, COL10, FAP, B7H3, LRRC15, and fibronectin-EDB isoform.

In some embodiments, the first target antigen is an antigen associated with the extracellular matrix of the tumor selected from nectin (e.g., nectin-3 or nectin-4), versican (VACN), fibronectin and a carcinoembryonic antigen-related cell adhesion molecules (CEACAM) protein.

In some embodiments, the tumor microenvironment of the tumor comprises cells that express the target antigen.

In some embodiments, the first target antigen is an antigen expressed on the surface of a cancer-associated fibroblast (e.g., α-smooth muscle actin (α-SMA), fibroblast activation protein (FAP), S100A4, platelet-derived growth factor receptors (PDGFRα/β), vimentin, PDPN, CD70, CD10, GPR77, CD10, CD74, CD146, CAV1, Saa3-, or CD49e).

In some embodiments, the first target antigen is an antigen expressed on the surface of a blood vessel in the tumor microenvironment, such as DLK1, EphA2, HBB, NG2, NRP1, NRP2, PDGFRβ, PSMA, RGS5, TEM1, VEGFR1 and VEGFR2.

In some embodiments, the first target antigen is an immune antigen. In some embodiments, the immune antigen is an antigen expressed on the surface of an immune cell. The immune cell may be a macrophage, a neutrophil, an eosinophil, a basophil, a mast cell, a monocyte, a dendritic cell, a natural killer cell, a T cell or a B cell. In some embodiments, the immune cell has infiltrated the tumor or tumor microenvironment of the tumor. The immune antigen may be selected from any one of the immune antigens listed in Table 4.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the tumor cells in the tumor do not express the first or second target antigen. In some embodiments, at least 10%400%, 20%-100%, 30%400%, 40%-100%, 50%-100%, 60%-100%, 70%-100%, 80%-100% or 90%-100% of the tumor cells in the tumor do not express the first and/or second target antigen.

The first target antigen may be selected from any one of the antigens listed in Table 2. The first target antigen may be CD22. The first target antigen may be a non-immune antigen. The non-immune antigen may be a CD38, EGFR, MUC16, PSMA, CA9, FOLR1, HER2, or SLAMF7. In some embodiments, the first multispecific antigen binding molecule is a bispecific antibody or a bispecific antibody fragment, such as a bispecific T-engaging antibody (BiTE), a dual-affinity re-targeting molecule (DART), and a tandem diabody (TandAb). In some embodiments, the first multispecific antigen binding molecule is selected from the bispecific antibodies listed in Table 3. In some embodiments, the second target antigen is selected from any one of the antigens listed in Table 2. The second target antigen may be CD20. The second target antigen may be an immune antigen. In some embodiments, the immune antigen is CD22, CD20, CD72, CD19, CD21, CD24, or CD79. The second multispecific antigen binding molecule may be a bispecific antibody or a bispecific antibody fragment. In some embodiments, the second multispecific antigen binding molecule is selected from the bispecific antibodies listed in Table 5. In some embodiments, the second multispecific antigen binding molecule is a bispecific antibody or a bispecific antibody fragment, such as a bispecific T-engaging antibody (BiTE), a dual-affinity re-targeting molecule (DART), and a tandem diabody (TandAb).

In some embodiments, the first multispecific antigen binding molecule demonstrates a costimulatory effect when administered conjointly with the second multispecific antigen binding molecule. The costimulatory effect may be one or more of the following: activating T-cells, inducing IL-2 release, inducing CD25+ up-regulation in PBMCs, and increasing T-cell mediated cytotoxicity.

The tumor may be from a B cell cancer, such as diffuse large B-cell lymphoma (DLBCL), follicular lymphoma, chronic lymphocytic leukemia (CLL), small lymphocytic lymphoma (SLL), mantle cell lymphoma (MCL), marginal zone lymphomas, burkitt lymphoma, lymphoplasmacytic lymphoma (Waldenstrom macroglobulinemia), hairy cell leukemia, primary central nervous system (CNS) lymphoma, or primary intraocular lymphoma (lymphoma of the eye).

In some embodiments, the tumor is a solid tumor. The tumor may be an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngreal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

In some embodiments, the first multispecific antigen binding molecule is administered systemically, intravenously, subcutaneously, or intramuscularly. The second multispecific antigen binding molecule may be administered systemically, intravenously, subcutaneously, or intramuscularly. The first multispecific antigen binding molecule and/or the second multispecific antigen binding molecule may be administered to the subject in a pharmaceutically acceptable formulation.

In some embodiments, the method further comprises administering an additional anti-cancer agent. In some embodiments, the additional anti-cancer agent is a chemotherapeutic agent, an immune checkpoint inhibitor, CAR-T cells, or a tumor vaccine. In some embodiments, the additional anti-cancer agent is an immune checkpoint inhibitor. The immune checkpoint inhibitor is an anti-PD-1 antibody, an anti-PDL1 antibody, an anti-CTLA4 antibody, or an anti-LAG3 antibody.

In some embodiments, the subject may be afflicted with a refractory cancer.

### BRIEF DESCRIPTION OF THE DRAWINGS

FIG. 1A-FIG. 1C show CD28 is expressed on intratumoral CD8 T cells from r/r NHL patients both prior to and post odronextamab treatment. FIG. 1A shows a representative image of multiplex IHC staining (CD3, CD8, CD28, and DAPI) of a baseline DLBCL patient sample from a odronextamab phase I study. FIG. 1B shows density of CD4+CD28+ and CD8+CD28+ cells from baseline DLBCL and FL samples. N=64. FIG. 1C shows representative images of DLBCL patient samples from baseline (top left) and 5 weeks post the start of odronextamab treatment (top right). Density of CD8+Cd28+ and CD8+CD28+ cells from paired DLBCL and FL sample at baseline and 5 weeks on treatment.

FIG. 2A-FIG. 2H shows REGN5837 bispecific antibody enhances odronextamab mediated T cell activation, cytotoxicity, and effector function in vitro. WSU-DLCL2 cells were incubated with lymphocyte-enriched human PBMC with a dose titration of odronextamab and a fixed concentration of REGN5837 (ranging from 7.72×10-″ to 1.00×10⁻⁷ M). FIG. 2A shows tumor cell killing was calculated by percent dead cells. FIG. 2B and FIG. 2D shows activation of CD4 and CD8 T cells was represented by CD25 upregulation. FIG. 2C and FIG. 2E shows proliferation was calculated by percent divided of CD4 and CD8 T cells. FIG. 2F shows supernatants were assessed for cytokine release of IL-2, IL-4, IL-6, IL-10, TNF-α, IFN-γ, and IL-17A. Arrows indicate fold change of EC50 or fold change of max cytokine concentration between highest concentration of REGN5837 (1.00×10⁻⁷M) to no REGN5837. FIG. 2G shows REGN5837 (R5837) augmentation of T cell killing of CD22 negative cells was determined by incubating purified human T cells with varying ratios of mixed CD22+ and CD22-WSU-DLCL2 tumor cells along with a fixed concentration of REGN5837 (ranging from 4.63×10⁻¹⁰ to 1.67×10⁻⁸M) and 5 pM of odronextamab. Killing of CD22+ targets is depicted in shades of red while killing of the CD22−targets is depicted in shades of blue. FIG. 2H shows activation of T cells in the mixed WSU-DLCL2 CD22+ and CD22-cultures was determined by upregulation of CD25.

FIG. 3A-FIG. 3F show REGN5837 enhances odronextamab anti-tumor efficacy and expands intratumoral CD8 T cells in a WSU-DLCL2 tumor model in a prophylactic treatment setting. FIG. 3A shows treatment schema for WSU-DLCL2 tumors implanted into NSG animals. FIG. 3B shows individual tumor volumes are plotted. Ratios indicate the number of tumor free mice. FIG. 3C shows average tumor growth (left) and survival (right). Statistical significance for average tumor growth was calculated with 2 way ANOVA and Tukey's multiple comparisons. Statistical significance for survival was calculated using the Kaplan-Meier method with log-rank test. *P<0.05, “P<0.01, ***P<0.001. WSU-DLCL2 tumor bearing animals were sacrificed 26 days post implantation to immunophenotype intratumoral T cell responses. UMAP plot of all live cells from the tumor was overlaid with color-coded immune cell subsets identified by FlowSOM (FIG. 3D left) and density UMAP plots (FIG. 3D right) revealed skewing of certain populations in response to combination treatment. FIG. 3E shows density of WSU-DLCL2 cells (left) and density of intratumoral CD8+ T cells (right). FIG. 3F shows pie charts depicting proportions of activated and memory subsets for intratumoral CD8 T cells in response to treatment (left). Density of effector memory and central memory intratumoral CD8+ T cells (right). Statistics were calculated with 1 way ANOVA with Tukey's test. ***P<0.001, ****P<0.0001

FIG. 4A-FIG. 4F show REGN5837 mediated co-stimulation enhances odronextamab anti-tumor efficacy against B cells malignancies in a therapeutic treatment setting. FIG. 4A shows treatment schema for therapeutic treatment of WSU-DLCL2 tumors implanted into NSG animals. FIG. 4B shows individual tumor volumes are plotted. Ratios indicate the number of tumor free mice. FIG. 4C shows average tumor growth (left) and Survival (right). Statistical significance for average tumor growth was calculated with 2 way ANOVA and Tukey's multiple comparisons. Statistical significance for survival was calculated using the Kaplan-Meier method with log-rank test. *P<0.05, **P<0.01. FIG. 4D shows treatment schema for therapeutic treatment of NALM6-luc tumors implanted into PBMC engrafted NSG animals. FIG. 4E shows BLI showing tumor burden in individual mice. FIG. 4F shows average NALM6-luc tumor growth. Significance was calculated with 2way ANOVA and Tukey's multiple comparisons. *P<0.05, ***P<0.001.

FIG. 5A-FIG. 5J shows a combination of REGN5837 with odronextamab enhances peripheral and intratumoral T cell responses in human immune system reconstituted animals bearing WSU-DLCL2 tumors. FIG. 5A shows treatment schema for WSU-DLCL2 tumors implanted into human immune system reconstituted animals. FIG. 5B shows individual tumor volumes are plotted. FIG. 5C shows average tumor growth (left) and survival (right). Statistical significance for average tumor growth was calculated with 2 way ANOVA and Tukey's multiple comparisons. Statistical significance for survival was calculated using the Kaplan-Meier method with log-rank test. *P<0.05, “P<0.01. FIG. 5D shows time course of peripheral CD8 T cell counts (left) and peripheral B cell counts (right) in response to treatment. FIG. 5E shows time course of serum cytokines induced in response to treatment. WSU-DLCL2 tumor bearing human immune system animals were sacrificed 30 days post implantation to immunophenotype intratumoral T cell responses. UMAP plot of all live cells from the blood, spleen, and tumor was overlaid with color-coded immune cell subsets identified by FlowSOM (FIG. 5F left) and density UMAP plots (FIG. 5F right) revealed skewing of certain populations in response to combination treatment. FIG. 5G shows density of intratumoral CD4 T cells (top right), CD8 T cells (top left), and WSU-DLCL2 cells (bottom). FIG. 5H shows UMAP plot of all intratumoral T cells was overlaid with color-coded metaclusters identified by FlowSOM (left) and density UMAP plots (right) revealed skewing of certain metaclusters. FIG. 5I shows a heat map of T cell activation, memory, dysfunction markers used by FlowSOM to identify T cell metaclusters. FIG. 5I shows frequencies of selected T cell clusters that are enriched or decreased in response to combination treatment. Statistics were calculated with 1 way ANOVA with Tukey's test. *P<0.05, “P<0.01, ****P<0.0001.

FIG. 6A-FIG. 6D show synergistic activation of CD8 T cells in the peripheral blood of cynomolgus monkeys when REGN5837 is combined with odronextamab. Cynomolgus monkeys received a single dose of REGN5837 at either 1 or 10 mg/kg (indicated in parentheses) in combination with a dose titration of Odronextamab. Blood was collected at the indicated times after dose (hours). FIG. 6A shows B cell counts at 5 hrs post dosing (left) and for duration of experiment (right). FIG. 6B shows peripheral CD8+ T cell counts at 5 hrs post dosing (left) and for the duration of the experiment (right). FIG. 6C shows ICOS upregulation on peripheral CD8+ T cells at 5 hrs post dosing (left) and proliferation (right) at 4 days post dosing. FIG. 6D shows serum cytokines induced 5 hours post dosing. Statistics were calculated with 1 way ANOVA with Tukey's test. Black stars indicates significance in comparison to placebo. n=3 animals/group. *P<0.05, **13<0.01, ***13<0.001, ****P<0.0001.

FIG. 7A-FIG. 7C shows expression of CD22 expression is variable on resected DLBCL patient samples. FIG. 7A shows chromogenic immunohistochemical staining for CD20 and CD22 on resected treatment naïve DLBCL patient samples. Representative images of high (left), medium (middle), and low (right) CD22 expression. FIG. 7B shows representative images of DLBCL patient samples with high (top) and low (bottom) % of CD22 expression from multiplex IHC staining for PAX5, CD20, and CD22. FIG. 7C shows percentage of B cell marker positive cells from treatment naïve DLBCL patient samples.

FIG. 8A-FIG. 8B show CD28, CTLA4, CD80 and CD86 are detectable DLBCL patient samples by chromogenic IHC. FIG. 8A shows representative images of chromogenic staining for CD28, CTLA4, CD80, and CD86 on treatment naïve resected DLBCL patient samples (Tristar). FIG. 8B shows density of CD28′, CTLA4′, CD86′, and CD80′ cells from 25 DLBCL patient samples.

FIG. 9A-FIG. 9D show REGN5837 bispecific antibody potentiates odronextamab mediated T cell cytotoxicity and proliferation. FIG. 9A shows REGN5837, non-targeted control antibodies, or a CD28 superagonist (REGN2329) were anchored to assay plates using the wet-coating method. Human PBMCs were incubated in the antibody coated assay plates and cytokine release measured after 50-54 hours. Data shown are from 4 individual donors. Cytokines for which a significant release response is observed compared with the non-binding control group are indicated (Tukey's post hoc test). FIG. 9B shows a number of CD22 (left) and CD20 (right) epitopes per cell on the WSU-DLCL2 cell line reported as antibody binding capacity. FIG. 9C shows a summary of REGN5837 mediated human CD4 and CD8 T cell proliferation in the presence or absence of odronextamab when cultured with NALM-6 or Raji CD80/CD86 DKO cells. NC, not calculated. FIG. 9D shows CD22 (left) and CD20 (right) expression by flow cytometry on WSU-DLCL2 CD22 ko (red) and WSU-DLCL2 CD22 wt (green) lines.

FIG. 10A-FIG. 10D show odronextamab monotherapy treatment suppresses tumor growth but does not mediate complete tumor rejection in a WSU-DLBCL (DLBCL) tumor model. FIG. 10A shows treatment schema for WSU-DLCL2 tumors implanted into NSG animals. FIG. 10B shows individual tumor growth curves in response to dose titration of odronextamab. FIG. 10C shows average tumor growth and FIG. 10D shows survival. Statistical significance for survival was calculated using the Kaplan-Meier method with log-rank test between isotype treatment and R1979 titration. ***P<0.001, ****P<0.0001.

FIG. 11A-FIG. 11E show REGN5837 in combination with odronextamab promotes intratumoral T cell expansion and activation and the killing of WSU-DLBCL2 tumor cells in vivo. FIG. 11A shows treatment schema for immunophenotyping of WSU-DLCL2 tumors in NSG mice at 26 days post implantation. FIG. 11B shows tumor mass plotted for each treatment group at 26 days post implantation. FIG. 11C shows the percentage of indicated cell population from total live cells demonstrating an expansion of CD4 and CD8 T cells and a skewing away from WSU-DLCL2 cells. FIG. 11D shows the density of intratumoral CD4 T cells plotted. FIG. 11E shows percentage of memory subsets plotted for intratumoral CD4 T cells. Statistics were calculated with 1 way ANOVA with Tukey's test. *P<0.05, **P<0.01, ***P<0.001, ****P<0.0001.

FIG. 12A-FIG. 12B show REGN5837 augments IL-2 production and enhancement of T cell proliferation in the presence of signal 1 mediated by an allogenic response of by odronextamab. The capacity of REGN5837 to mediate IL-2 release and T-cell proliferation in the presence of a human B-cell leukemia cell line was determined using enriched human primary T cells and allogeneic NALM-6 cells. FIG. 12A shows assays were performed in the presence of odronextamab (500 pM) to provide signal 1, or in the absence of odronextamab where signal 1 was provided solely by the allogeneic response and a dose titration of REGN5837 or a non-binding CD28 bispecific control antibody. Plates were incubated for 72 hours at which time the culture supernatant was collected for IL-2 analysis. To assess proliferation, tritium was added and the cells incubated for an additional 16 hours. FIG. 12B shows a summary of REGN5837 mediated concentration-dependent increases in IL-2 release and T-cell proliferation in the presence and absence of odronextamab.

FIG. 13A-FIG. 13H show that a combination of REGN5837 with odronextamab expands peripheral and intratumoral T cells in human immune system reconstituted animals bearing DLBCL tumors. FIG. 13A shows a time course of peripheral CD4 T cell counts. FIG. 13B shows a time course of serum IL-10 induced in response to treatment. FIG. 13C shows average WSU-DLCL2 tumor growth prior to sacrifice for immunophenotyping. Statistical significance was calculated with 2 way ANOVA and Tukey's multiple comparisons between combination treatment and isotype (black stars) or combination treatment and REGN5837 monotherapy (red stars). FIG. 13D shows a percentage of WSU-DLCL2 cells (left) and T cells (right) from all live intratumoral cells. FIG. 13E and FIG. 13F shows the percentage of memory and activated subsets of intratumoral CD8 (FIG. 13E) and CD4 (FIG. 13F) T cells. FIG. 13G and FIG. 13H show the intratumoral density of memory CD8 (FIG. 13G) and CD4 (FIG. 13H) T cells. Statistics were calculated with 1 way ANOVA with Tukey's test. *P<0.05, **P<0.01, ***1)<0.001, ****P<0.0001.

FIG. 14A-FIG. 14E show odronextamab efficiently depletes splenic and blood B cells while promoting effector memory T cell induction in human immune system reconstituted animals bearing DLBCL tumors. FIG. 14A and FIG. 14B show a percentage of B cells (left) and T cells (right) from spleen (FIG. 14A) or blood (FIG. 14B) at 30 days post implantation of WSU-DLCL2 tumors. FIG. 14C shows enumeration of B and T cells subsets in the spleen. FIG. 14D shows density UMAP plots (right) of all live cells in the blood. FIG. 14E shows enumeration of B and T cells subsets in the blood.

FIG. 15A-FIG. 15B show synergistic activation of CD4 T cells in peripheral blood of cynomolgus monkeys when REGN5837 is combined with odronextamab. FIG. 15A shows peripheral CD4 T cell counts at 4 days post dosing (left) and for the duration of the experiment. FIG. 15B shows ICOS upregulation on peripheral CD4 T cells at 5 hours post dosing (left) and proliferation (right) at 4 days post dosing. Statistics were calculated with one-way ANOVA with Tukey's test. Black stars indicates significance in comparison to placebo. n=3 animals/group. *P<0.05, “P<0.01, ***P<0.001, ****P<0.0001.

FIG. 16 shows CD22 expression is more variable than CD20 expression in DLBCL patient samples.

FIG. 17 shows that, in a mixed culture of CD22 wt and CD22ko tumor cells, CD22×CD28 combined with CD20×CD3 can mediate enhanced T cell killing of bystander CD22ko target cells.

FIG. 18 shows an exemplary expression profile for EGFR target antigen in several cancers.

FIG. 19 shows an exemplary expression profile for PMSA target antigen in several cancers.

FIG. 20 shows an exemplary expression profile for CA9 target antigen in several cancers.

FIG. 21 shows an exemplary expression profile for HER2 target antigen in several cancers.

FIG. 22 shows an exemplary expression profile for SLAMF7 target antigen in several cancers.

FIG. 23 shows an exemplary expression profile for MUC16 target antigen in several cancers.

FIG. 24 shows an exemplary expression profile for FOLR1 target antigen in sever cancers.

FIG. 25 shows an exemplary expression profile for CD38 target antigen in several cancers.

FIG. 26 shows an exemplary expression profile for CD22 target antigen in several cancers.

### DETAILED DESCRIPTION

As shown herein, culturing T cells with target cells expressing CD22⁺ and an anti-CD28/anti-CD22 bispecific antibody augmented CD3 bispecific-mediated tumor cell lysis. The disclosure herein is based, in part, on the discovery that combination treatment with anti-CD28/anti-CD22 bispecific antibody increased CD3 bispecific-mediated tumor cell lysis of both the CD22⁺ and CD22⁻ target populations. Similar methods also increased CD4 and CD8 T cell activation in both the CD22+ and CD22⁻ target populations. Therefore, Applicant shows that anti-CD28 bispecific antibodies targeting a tumor associated antigen can increase killing of tumor cells lacking the tumor associated antigen.

Provided herein are methods and compositions for mediating killing of a tumor cell in a tumor in a subject by administering to the subject a multispecific antigen binding molecule with a first antigen binding region specific for a target antigen and a second antigen binding region specific for a CD28 protein, wherein the tumor cell does not express or is not predicted to express the target antigen. In some embodiments, at least a subset of tumor cells in the tumor do not express the target antigen.

In some aspects, provided herein are methods and compositions for inducing killing of tumor cells and/or inducing T cell activation against tumor cells in a tumor in a subject, the method comprising administering to the subject a multispecific antigen binding molecule with a first antigen binding region specific for a target antigen and a second antigen binding region specific for a CD28 protein. In some embodiments, at least a subset of tumor cells in the tumor do not express the target antigen.

Also provided herein are methods and compositions for treating cancer in a subject with a tumor, the method comprising administering to the subject a multispecific antigen binding molecule with a first antigen binding region specific for a target antigen and a second antigen binding region specific for a CD28 protein. In some embodiments, at least a subset of tumor cells in the tumor do not express the target antigen.

As used herein, when referring to tumor cells in the tumor that “do not express the target antigen”, the phrase includes experimental detection or confirmation that at least a subset of tumor cells do not express the target antigen, as well as methods involved predicting (i.e., with or without further experimental confirmation) that the tumor cells do not express the target antigen.

In some embodiments, the target antigen is a tumor associated antigen (TAA). In some embodiments, the target antigen is an antigen associated with a tumor microenvironment (e.g., the microenvironment of the tumor in the subject). For example, in some embodiments the target antigen is an antigen on an immune cell, on tumor cell stroma, or on the extracellular matrix within the tumor microenvironment. Examples of extracellular matrix antigens include nectin (e.g., nectin-3 or nectin-4), versican (VACN), fibronectin and carcinoembryonic antigen-related cell adhesion molecules (CEACAM).

### Definitions

The articles “a” and “an” are used herein to refer to one or to more than one (i.e., to at least one) of the grammatical object of the article. By way of example, “an element” means one element or more than one element.

As used herein, the term “administering” or “administration” means providing a pharmaceutical agent or composition to a subject, and includes, but is not limited to, administering by a medical professional and self-administering. Such an agent can contain, for example, a bispecific antibody or a bispecific antibody fragment provided herein.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies, trispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F(ab′)2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins, Fynomers®, Centyrins®, and KALBITOR®s, CARs, engineered TCRs, and antigen-binding fragments of any of the above.

An antibody for use in the instant invention may be a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences.

Antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the disclosure may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

As used herein, a “cancer-associated fibroblasts” (CAFs) include fibroblasts found within and surrounding tumor tissues, which are activated from normal resident tissue fibroblasts or transdifferentiated from non-fibroblastic lineage such as epithelial cells and adipocytes due to the stimulation of tumor microenvironment. Exemplary cancer associated fibroblast antigens include α-smooth muscle actin (α-SMA), fibroblast activation protein (FAP), S100A4, platelet-derived growth factor receptors (PDGFRα/β), vimentin, PDPN, CD70, Cd10, GPR77, CD10, CD74, CD146, CAV1, Saa3-, and CD49e. More details regarding antigens and biomarkers can be found in Han, C., Liu, T. & Yin, R. Biomarkers for cancer-associated fibroblasts. Biomark Res 8, 64 (2020).

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s) and” “neoplasm(s)”” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

The term “chimeric antigen receptor” (CAR) refers to molecules that combine a binding domain against a component present on the target cell, for example an antibody-based specificity for a desired antigen (e.g., a tumor antigen) with a T cell receptor-activating intracellular domain to generate a chimeric protein that exhibits a specific anti-target cellular immune activity. Generally, CARs consist of an extracellular single chain antigen-binding domain (scFv) fused to the intracellular signaling domain of the T cell antigen receptor complex zeta chain, and have the ability, when expressed in T cells, to redirect antigen recognition based on the monoclonal antibody's specificity.

As used herein, the phrase “conjoint administration” or “administered conjointly” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

A “costimulatory domain” or “costimulatory molecule” refers to the cognate binding partner on a T-cell that specifically binds with a costimulatory ligand, thereby mediating a costimulatory response by the cell, such as, but not limited to proliferation. The costimulatory domain may be a human costimulatory domain. Exemplary costimulatory molecules include, CD28, 4-1BB, CD27, CD8, 4-1BB (CD137), OX40, CD30, CD40, PD-1, ICOS, lymphocyte function-associated antigen-1 (LFA-1), CD2, CD7, LIGHT, NKG2C, and B7-H3.

A “costimulatory ligand” refers to a molecule on an antigen presenting cell that specifically binds a cognate costimulatory molecule on a T-cell, thereby providing a signal which mediates a T cell response, including, but not limited to, proliferation activation, differentiation and the like. A costimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3.

A “costimulatory signal” refers to a signal, which in combination with a primary signal, leads to T cell proliferation and/or upregulation or downregulation of key molecules.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

As used herein, “immune cell” includes any white blood cell that developed from stem cells in bone marrow. Examples include macrophages, neutrophils, eosinophils, basophils, mast cells, monocytes, dendritic cells, natural killer cells, T cells or B cells. Immune cells can be present in the tumor or tumor microenvironment.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

The term “specifically binds” or “specific binding”, as used herein, when referring to a polypeptide refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M¹, 10⁷ M⁻¹, 10⁸ M⁻, 10⁹ M⁻¹, 10¹⁰ M⁻¹, 10¹¹ M⁻¹, and 10¹² M⁻¹ or more) with that second molecule. For example, in the case of the ability of a PIG-specific CAR to bind to a peptide presented on an MHC (e.g., class I MHC or class II MHC); typically, a CAR specifically binds to its peptide/MHC with an affinity of at least a KD of about 10-4 M or less, and binds to the predetermined antigen/binding partner with an affinity (as expressed by KD) that is at least 10 fold less, at least 100 fold less or at least 1000 fold less than its affinity for binding to a non-specific and unrelated peptide/MHC complex (e.g., one comprising a BSA peptide or a casein peptide).

As used herein, the term “subject” means a human or non-human animal selected for treatment or therapy. In certain embodiments provided herein the subject is a human subject. In some embodiments provided herein, the subject is a subject in need of a method provided herein, such as a subject who has cancer.

The “tumor microenvironment” refers to the cellular environment in which the tumor exists, and includes, for example, the stroma, interstitial fluids surrounding the tumor, surrounding blood vessels, immune cells, other cells, fibroblasts, signaling molecules, and the extracellular matrix.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

### Therapeutic Antibodies

### General

In certain aspects, the methods and compositions provided herein relate to the use of therapeutic antibodies (e.g., bispecific antibody disclosed herein, such as a bispecific antibody with an antigen binding region specific for a first target antigen and an antigen binding region specific for a CD28 protein, optionally administered conjointly with a second anti-CD3 bispecific antibody and/or an immune checkpoint inhibitor).

As set forth above, as used herein, the term “antibody” encompasses both full antibody molecules and antigen-binding fragments of full antibody molecules. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody disclosed herein include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody antibody disclosed herein may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific or trispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody disclosed herein using routine techniques available in the art.

In certain embodiments provided herein, at least one variable domain of a multispecific antibody is capable of specifically binding to a T cell co-stimulatory domain, such as CD28. In certain embodiments provided herein, at least one variable domain of a multispecific antibody disclosed herein is capable of specifically binding to CD3.

In some embodiments, the antibodies provided herein may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody disclosed herein in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments provided herein, the multispecific (e.g., bispecific or trispecific) antibodies provided herein are human antibodies. The term “human antibody,” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies provided herein, in some embodiments, may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, CH2 or CH3 region which may be desirable, for example, in production, to improve the yield of the desired antibody form.

### Sequence Variants

In some embodiments, the monospecific or multispecific (e.g., bispecific or trispecific) antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies disclosed herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding (e.g., as measured by cell binding titration or FACS binding) or binding affinity (e.g., KD), improved or enhanced antagonistic or agonistic biological properties (as the case may be), reduced immunogenicity, etc. Antibodies and antigen-binding fragments obtained in this general manner are encompassed within the present disclosure.

In some embodiments, the multispecific (e.g., bispecific or trispecific) antibodies provided herein comprise variants of any of the HCVR, LCVR, and/or CDR amino acid sequences disclosed herein having one or more conservative substitutions. For example, in certain embodiments the anti-CD40 antagonist antibodies or CD3 multispecific (e.g., bispecific or trispecific) antibodies provided herein have HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

### Fc Variants

According to certain embodiments provided herein, antibodies and multispecific antigen-binding molecules are provided comprising an Fc domain comprising one or more mutations which enhance or diminish antibody binding to the FcRn receptor, e.g., at acidic pH as compared to neutral pH. For example, the present disclosure includes antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the present disclosure includes multispecific antigen-binding molecules (e.g., anti-CD28/anti-TAA bispecific or anti-CD3/anti-TAA bispecific antibodies), comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated herein.

### Bioequivalents

Provided herein antigen-binding molecules having amino acid sequences that vary from those of the exemplary molecules disclosed herein but that retain the ability to bind the same antigen or antigens. Such variant molecules may comprise one or more additions, deletions, or substitutions of amino acids when compared to parent sequence, but exhibit biological activity that is essentially equivalent to that of the described bispecific antigen-binding molecules.

The present disclosure includes antigen-binding molecules that are bioequivalent to any of the exemplary antigen-binding molecules set forth herein. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

### Antibody Binding

As used herein, the term “binding” in the context of the binding of an antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a KD value of about 10-7 M or less, such as about 10-8 M or less, such as about 10-9 M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, CA, J Immunol Methods. 1997, 201(2):223-31; Geuijen, C A, et al. J Immunol Methods. 2005, 302(1-2):68-77).

Accordingly, the antibody or antigen-binding protein provided herein binds to the predetermined antigen or cell surface molecule (receptor) having an affinity corresponding to a KD value that is at least ten-fold lower than its affinity for binding to a non-specific antigen (e.g., BSA, casein). According to the present disclosure, the affinity of an antibody corresponding to a KD value that is equal to or less than ten-fold lower than a non-specific antigen may be considered non-detectable binding, however such an antibody may be paired with a second antigen binding arm for the production of a bispecific antibody disclosed herein.

The term “KD” (M) refers to the dissociation equilibrium constant of a particular antibody-antigen interaction, or the dissociation equilibrium constant of an antibody or antibody-binding fragment binding to an antigen. There is an inverse relationship between KD and binding affinity, therefore the smaller the KD value, the higher, i.e. stronger, the affinity. Thus, the terms “higher affinity” or “stronger affinity” relate to a higher ability to form an interaction and therefore a smaller KD value, and conversely the terms “lower affinity” or “weaker affinity” relate to a lower ability to form an interaction and therefore a larger KD value. In some circumstances, a higher binding affinity (or KD) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger KD value (lower, or weaker, affinity) by the smaller KD (higher, or stronger, affinity), for example expressed as 5-fold or 10-fold greater binding affinity, as the case may be.

The term “kd” (sec −1 or 1/s) refers to the dissociation rate constant of a particular antibody-antigen interaction, or the dissociation rate constant of an antibody or antibody-binding fragment. Said value is also referred to as the koff value.

The term “ka” (M−1×sec−1 or 1/M) refers to the association rate constant of a particular antibody-antigen interaction, or the association rate constant of an antibody or antibody-binding fragment.

The term “KA” (M−1 or 1/M) refers to the association equilibrium constant of a particular antibody-antigen interaction, or the association equilibrium constant of an antibody or antibody-binding fragment. The association equilibrium constant is obtained by dividing the ka by the kd.

The term “EC50” or “EC50” refers to the half maximal effective concentration, which includes the concentration of an antibody which induces a response halfway between the baseline and maximum after a specified exposure time. The EC50 essentially represents the concentration of an antibody where 50% of its maximal effect is observed. In certain embodiments, the EC50 value equals the concentration of an antibody disclosed herein that gives half-maximal binding to cells expressing CD28 or tumor-associated antigen (e.g., a antigen disclosed in Table 2), as determined by e.g. a FACS binding assay. Thus, reduced or weaker binding is observed with an increased EC50, or half maximal effective concentration value.

In one embodiment, decreased binding of CD28 multispecific antibodies can be defined as an increased EC50 antibody concentration which enables binding to the half-maximal amount of target cells.

In another embodiment, the EC50 value represents the concentration of a CD28 multispecific antibody disclosed herein that elicits half-maximal depletion of target cells by T cell cytotoxic activity. Thus, increased cytotoxic activity (e.g. T cell-mediated tumor cell killing) is observed with a decreased EC50, or half maximal effective concentration value.

### Preparation of Antigen-Binding Domains and Construction of Multispecific Molecules

Antigen-binding domains specific for particular antigens can be prepared by any antibody generating technology known in the art. Once obtained, two different antigen-binding domains, specific for two different antigens (e.g., CD28 and a human tumor antigen), can be appropriately arranged relative to one another to produce a bispecific antigen-binding molecule disclosed herein using routine methods. In certain embodiments, one or more of the individual components (e.g., heavy and light chains) of the multispecific antigen-binding molecules disclosed herein are derived from chimeric, humanized or fully human antibodies. Methods for making such antibodies are well known in the art. For example, one or more of the heavy and/or light chains of the bispecific antigen-binding molecules disclosed herein can be prepared using VELOCIMMUNE™ technology. Using VELOCIMMUNE™ technology (or any other human antibody generating technology), high affinity chimeric antibodies to a particular antigen (e.g., CD28 or human tumor associated antigen) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules disclosed herein.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454). Fully human refers to an antibody, or antigen-binding fragment or immunoglobulin domain thereof, comprising an amino acid sequence encoded by a DNA derived from a human sequence over the entire length of each polypeptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g. compared to any wild-type human immunoglobulin regions or domains.

### CD28 Multispecific Antigen-Binding Molecules

In certain embodiments, the methods and compositions provided herein relate to CD28 antigen-binding molecules (i.e., antigen binding molecules that comprise at least one antigen binding region that binds to CD28). In certain embodiments, the CD28 multispecific antigen-binding molecules provided herein further comprise an antigen binding domain that binds to a target antigen (e.g., an antigen expressed on a cancer cell, such as a tumor associated antigen (TAA)).

In certain embodiments, the provided herein are second antigen binding molecules that are CD3 multispecific antigen-binding molecules provided herein further comprise an antigen binding domain that binds to a target antigen (e.g., an antigen expressed on a cancer cell, such as a tumor associated antigen (TAA)).

In some embodiments, the target antigen is a tumor associated antigen (TAA). In some embodiments, the target antigen is an antigen associated with a tumor microenvironment (e.g., the microenvironment of the tumor in the subject). For example, in some embodiments the target antigen is an antigen on an immune cell, on tumor cell stroma, or on the extracellular matrix within the tumor microenvironment. Examples of extracellular matrix antigens include nectin (e.g., nectin-3 or nectin-4), versican (VACN), fibronectin and carcinoembryonic antigen-related cell adhesion molecules (CEACAM).

As used herein, the expression “multispecific antigen-binding molecule” refers to a protein, polypeptide or molecular complex comprising at least a first antigen-binding region and a second antigen-binding region. In some embodiments, each antigen-binding domain within the multispecific antigen-binding molecule may comprises at least one CDR that alone, or in combination with one or more additional CDRs and/or FRs, specifically binds to a particular antigen. In certain embodiments, the first antigen-binding domain specifically binds a first antigen (e.g., CD28), and the second antigen-binding domain specifically binds a second, distinct antigen (e.g., a tumor associated antigen).

In some embodiments, the CD28 multispecific antigen-binding molecule is an CD28 multispecific antibody, such as a CD28 bispecific antibody. The CD28 multispecific antibodies of provided herein may be, for example, bi-specific, or tri-specific. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The CD28 bispecific antibodies provided herein can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second or additional binding specificity.

The term “CD28,” as used herein, refers to an Cluster of Differentiation 28, which is one of the proteins expressed on T cells that provide co-stimulatory signals required for T cell activation and survival. T cell stimulation through CD28 in addition to the T-cell receptor (TCR) can provide a potent signal for the production of various interleukins. Human CD28 comprises the amino acid sequence as set forth below.

TABLE 1
Human Protein Sequences of CD28

>NP_001230006.1; GeneID = 940; isoform = 2 precursor
MLRLLLALNLFPSIQVTGNKILVKQSPMLVAYDNAVNLSWKHLCPSPLFPGPSKPFWVLVVVGGVLACYSLL
VTVAFIIFWVRSKRSRLLHSDYMNMTPRRPGPTRKHYQPYAPPRDFAAYRS
>NP_001230007.1; GeneID = 940; isoform = 3 precursor
MLRLLLALNLFPSIQVTGKHLCPSPLFPGPSKPFWVLVVVGGVLACYSLLVTVAFIIFWVRSKRSRLLHSDY
MNMTPRRPGPTRKHYQPYAPPRDFAAYRS
>NP_006130.1; GeneID = 940; isoform = 1 precursor
MLRLLLALNLFPSIQVTGNKILVKQSPMLVAYDNAVNLSCKYSYNLFSREFRASLHKGLDSAVEVCVVYGNY
SQQLQVYSKTGFNCDGKLGNESVTFYLQNLYVNQTDIYFCKIEVMYPPPYLDNEKSNGTIIHVKGKHLCPSP
LFPGPSKPFWVLVVVGGVLACYSLLVTVAFIIFWVRSKRSRLLHSDYMNMTPRRPGPTRKHYQPYA
PPRDFAAYRS
>XP_011510496.1; GeneID = 940; isoform = X1
MPCGLSALIMCPKGMVAVVVAVDDGDSQALAGNKILVKQSPMLVAYDNAVNLSCKYSYNLFSREFRASLHKG
LDSAVEVCVVYGNYSQQLQVYSKTGFNCDGKLGNESVTFYLQNLYVNQTDIYFCKIEVMYPPPYLDNEKSNG
TIIHVKGKHLCPSPLFPGPSKPFWVLVVVGGVLACYSLLVTVAFIIFWVRSKRSRLLHSDYMNMTP
RRPGPTRKHYQPYAPPRDFAAYRS
>XP_011510497.1; GeneID = 940; isoform = X2
MPCGLSALIMCPKGMVAVVVAVDDGDSQALAGNKILVKQSPMLVAYDNAVNLSYNEKSNGTIIHVKGKHLCP
SPLFPGPSKPFWVLVVVGGVLACYSLLVTVAFIIFWVRSKRSRLLHSDYMNMTPRRPGPTRKHYQPYAPPRD
FAAYRS
>XP_011510499.1; GeneID = 940; isoform = X3
MLRLLLALNLFPSIQVTGNKILVKQSPMLVAYDNAVNLSYNEKSNGTIIHVKGKHLCPSPLFPGPSKPFWVL
VVVGGVLACYSLLVTVAFIIFWVRSKRSRLLHSDYMNMTPRRPGPTRKHYQPYAPPRDFAAYRS

All references to proteins, polypeptides and protein fragments herein are intended to refer to the human version of the respective protein, polypeptide or protein fragment unless explicitly specified as being from a non-human species. Thus, the expression “CD28” means human CD28 unless specified as being from a non-human species, e.g., “mouse CD28,” “monkey CD28,” etc.

As used herein, the expression “cell surface-expressed CD28” means one or more CD28 protein(s) that is/are expressed on the surface of a cell in vitro or in vivo, such that at least a portion of a CD28 protein is exposed to the extracellular side of the cell membrane and is accessible to an antigen-binding portion of an antibody. Cell surface-expressed CD28 includes CD28 proteins contained within the context of a functional T cell receptor in the membrane of a cell. A cell surface-expressed CD28 can comprise or consist of a CD28 protein expressed on the surface of a cell which normally expresses CD28 protein. Alternatively, cell surface-expressed CD28 can comprise or consist of CD3 protein expressed on the surface of a cell that normally does not express human CD28 on its surface but has been artificially engineered to express CD28 on its surface.

In some embodiments, the present disclosure includes bispecific antibodies wherein one arm of an immunoglobulin binds CD28, and the other arm of the immunoglobulin is specific for a target antigen (e.g., a tumor antigen, or “TAA”). In some embodiments, the present disclosure includes trispecific antibodies wherein a first arm of an immunoglobulin binds CD28, a second arm of the immunoglobulin is specific for a tumor antigen, and a third arm of the immunoglobulin binds an additional T cell antigen (e.g., CD3) or an additional tumor antigen.

In some embodiments, the CD28 multispecific antibody may comprise any of the antibodies disclosed in US 2020/0239576. In some embodiments, the CD28-binding arm may comprise any of the HCVR/LCVR or CDR amino acid sequences as disclosed in US 2020/0239576. In certain embodiments, the CD28-binding arm binds to human CD28 and induces human T cell activation. In certain embodiments, the CD28-binding arm binds weakly to human CD28 and induces human T cell activation. In other embodiments, the CD28-binding arm binds weakly to human CD28 and induces tumor-associated antigen-expressing cell killing in the context of a bispecific or multispecific antibody.

In certain embodiments, the multispecific antibodies or antigen-binding fragments for use in the present disclosure comprise an antigen-binding arm that binds to ICOS, HVEM, CD27, 4-1BB, OX40, DR3, GITR, CD30, SLAM, CD2, 2B4, CD226, TIM1, or TIM2 to induce T cell activation.

In certain embodiments, the CD28 multispecific antigen-binding molecule comprises an antigen-binding domain specific for a tumor associated antigen.

In certain embodiments, the tumor associated antigen is an immune tumor antigen. In certain embodiments, the tumor associated antigen is an non-immune tumor antigen.

The tumor associated antigen can be any one of the antigens selected from Table 2 below.

TABLE 2
Exemplary Tumor Associated Antigens

| | | | | |
|---|---|---|---|---|
| AIM-2 | ALDH1A1 | alpha-actinin-4 | alpha- | ARTC1 |
| | | | fetoprotein | |
| | | | (“AFP”) | |
| B-RAF | BAGE-1 | BCLX (L) | BCMA | BCR-ABL |
| | | | | fusion protein |
| | | | | b3a2 |
| beta-catenin | BING-4 | CA-125 | CALCA | carcinoembryon |
| | | | | ic antigen |
| | | | | (“CEA”) |
| CD9 | CASP-5 | CASP-8 | CD22 | CD20 |
| CD72 | CD19 | CD21 | CD24 | CD38 |
| CD79 | CD274 | CD45 | Cdc27 | CDK12 |
| CDK4 | CDKN2A | CEA | CLPP | COA-1 |
| CPSF | CSNK1A1 | CTAG1 | CTAG2 | cyclin D1 |
| Cyclin-A1 | dek-can fusion | DKK1 | EFTUD2 | Elongation |
| | protein | | | factor 2 |
| EGFR | ENAH (hMena) | Ep-CAM | EpCAM | EphA3 |
| epithelial | ETV6-AML1 | EZH2 | FOLR1 | FGF5 |
| tumor antigen | fusion protein | | | |
| (“ETA”) | | | | |
| FLT3-ITD | FN1 | G250/MN/CAIX | GAGE-1 | GAGE-2 |
| GAGE-8 | GAGE-3 | GAGE-4 | GAGE-5 | GAGE-6 |
| GAGE-7 | GAS7 | glypican-3 | GnTV | gp100/Pmel17 |
| GPNMB | HAUS3 | Hepsin | HER-2/neu | HERV-K-MEL |
| HLA-A11 | HLA-A2 | HLA-DOB | hsp70-2 | IDO1 |
| IGF2B3 | IL13Ralpha2 | Intestinal | K-ras | Kallikrein 4 |
| | | carboxyl | | |
| | | esterase | | |
| KIF20A | KK-LC-1 | KKLC1 | KM-HN-1 | KMHN1 also |
| | | | | known as |
| | | | | CCDC110 |
| LAGE-1 | LDLR- | Lengsin | M-CSF | MAGE-A1 |
| | fucosyltransferase | | | |
| | AS fusion protein | | | |
| MAGE-A10 | MAGE-A12 | MAGE-A2 | MAGE-A3 | MAGE-A4 |
| MAGE-A6 | MAGE-A9 | MAGE-C1 | MAGE-C2 | malic enzyme |
| mammaglobin-A | MART2 | MATN | MC1R | MCSP |
| mdm-2 | ME1 | Melan- | Meloe | Midkine |
| | | A/MART-1 | | |
| MMP-2 | MMP-7 | MUC1 | MUC5AC | MUC16 |
| mucin | MUM-1 | MUM-2 | MUM-3 | Myosin |
| Myosin class I | N-raw | NA88-A | neo-PAP | NFYC |
| NY-BR-1 | NY-ESO-1/ | OA1 | OGT | OS-9 |
| | LAGE-2 | | | |
| P polypeptide | p53 | PAP | PAX5 | PBF |
| pml- | polymorphic | PPP1R3B | PRAME | PRDX5 |
| RARalpha | epithelial mucin | | | |
| fusion protein | (“PEM”) | | | |
| PSA | PSMA | PTPRK | RAB38/N | RAGE-1 |
| | | | Y-MEL-1 | |
| RBAF600 | RGS5 | RhoC | RNF43 | RU2AS |
| SAGE | secernin 1 | SIRT2 | SNRPD1 | SOX10 |
| Sp17 | SPA17 | SLAMF7 | SSX-2 | SSX-4 |
| STEAP1 | STEAP2 | survivin | SYT-SSX1 | TAG-1 |
| | | | or -SSX2 | |
| | | | fusion | |
| | | | protein | |
| TAG-2 | Telomerase | TGF-betaRII | TPBG | TRAG-3 |
| Triosephosphate | TRP-1/gp75 | TRP-2 | TRP2- | tyrosinase |
| isomerase | | | INT2 | |
| tyrosinase | VEGF | WT1 and | | |
| (“TYR”) | | XAGE- | | |
| | | 1b/GAGED2a | | |

In certain embodiments, the tumor associated antigen is selected from AIM-2, ALDH1A1, alpha-actinin-4, alpha-fetoprotein (“AFP”), ARTC1, B-RAF, BAGE-1, BCLX (L), BCMA, BCR-ABL fusion protein b3a2, beta-catenin, BING-4, CA-125, CALCA, carcinoembryonic antigen (“CEA”), CASP-5, CASP-8, CD20, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUCSAC, MUC16, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, STEAP2, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1 and XAGE-1b/GAGED2a.

In some embodiments, the tumor associated antigen can include ADAM 17, BCMA, CA-IX, CD19, CD20, CD21, CD22, CD24, CD30, CD33, CD38, CD52, CD56, CD70, CD72, CD74, CD79b, CD123, CD138, CDH3, CEA, EphA2, EpCAM, ERBB2, ENPP3, EGFR, EGFR-vIII, FLT3, FOLR1, GD-2, glypican-3, gpA33, GPNMB, GPRC5D, HER2, HER3, LMP1, LMP2A, MUC16, Mesothelin, PSMA, PSCA, RON, ROR1, ROR2, STEAP1, STEAP2, SSTR2, SSTR5, 5T4, and Trop-2. In some embodiments, the tumor antigen may be CD19, CD123, STEAP2, CD20, SSTR2, CD38, STEAP1, 5T4, ENPP3, PSMA, MUC16, GPRC5D, or BCMA.

In some embodiments, the tumor associated antigen may be an non-immune associated antigen selected from CD38, EGFR, MUC16, PSMA, CA9, FOLR1, HER2, and SLAMF7.

In some embodiments, the tumor associated antigen may be an immune associated antigen selected from CD22, CD20, CD72, CD19, CD21, CD24, and CD79.

CD22 (Siglec 2) is a receptor expressed on the cell membranes of B cells. CD22 mediates B-cel/B-cell interactions. It also may be involved in the localization of B-cells in lymphoid tissues. CD22 binds sialylated glycoproteins; one of which is CD45. It also preferentially binds to alpha-2,6-linked sialic acid. It also plays a role in positive regulation through interaction with Src family tyrosine kinases and may also act as an inhibitory receptor by recruiting cytoplasmic phosphatases via their SH2 domains that block signal transduction through dephosphorylation of signaling molecules.

CD20 is a non-glycosylated phosphoprotein expressed on the cell membranes of mature B cells. CD20 is considered a B cell tumor-associated antigen because it is expressed by more than 95% of B-cell non-Hodgkin lymphomas (NHLs) and other B-cell malignancies, but it is absent on precursor B-cells, dendritic cells and plasma cells. The human CD20 protein has the amino acid sequence shown in SEQ ID NO: 5 of U.S. Patent Application Publication No. US 2020/0129617, the content of which is incorporated by reference herein in its entirety.

MUC16 refers to mucin 16. MUC16 is a single transmembrane domain highly glycosylated integral membrane glycoprotein that is highly expressed in ovarian cancer. The amino acid sequence of human MUC16 is set forth in SEQ ID NO:1899 of U.S. Patent Application Publication No. US 2018/0118848A1, the content of which is incorporated by reference herein in its entirety.

BCMA refers to B-cell maturation antigen. BCMA (also known as TNFRSF17 and CD269) is a cell surface protein expressed on malignant plasma cells, and plays a central role in regulating B cell maturation and differentiation into immunoglobulin-producing plasma cells. The amino acid sequence of human BCMA is shown in SEQ ID NO: 115 of U.S. Patent Application Publication No. US 2020/0024356, the content of which is incorporated by reference herein in its entirety. It can also be found in GenBank accession number NP_001183.2.

PSMA refers to prostate-specific membrane antigen, also known as folate hydrolase 1 (FOLH1). PSMA is an integral, non-shed membrane glycoprotein that is highly expressed in prostate epithelial cells and is a cell-surface marker for prostate cancer. The amino acid sequence of human PSMA is set forth in SEQ ID NO: 7 of U.S. Patent Application Publication No. US 2020/0129617, the content of which is incorporated by reference herein in its entirety.

In some embodiments, the CD28 multispecific antibody may be a bispecific CD28×CD19 antibody, a bispecific CD28×CD22 antibody, a bispecific CD28×CD20 antibody, a bispecific CD28×CD72 antibody, a bispecific CD28×CD20 antibody, a bispecific CD28×CD19 antibody, a bispecific CD28×CD21 antibody, a bispecific CD28×CD24 antibody, or a bispecific CD28×CD79 antibody.

In some embodiments, the CD28 multispecific antibody may be a bispecific CD28×CD38 antibody, a bispecific CD28×EGFR antibody, a bispecific CD28×MUC16 antibody, a bispecific CD28×PSMA antibody, a bispecific CD28×CA9 antibody, a bispecific CD28×CD20 antibody, a bispecific CD28×FOLR1 antibody, a bispecific CD28×HER2 antibody, and a bispecific CD28×SLAM7 antibody.

In certain embodiments, the mulitispecific antigen-binding molecule is a mulitispecific antibody or antigen-binding fragment thereof. Each antigen-binding domain of a mulitispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A 1-HCDR1, A 1-HCDR2, and A 1-HCDR3; and the CDRs of the second antigen-binding domain may be referred to herein as A2-HCDR1, A2-HCDR2, and A2-HCDR3. In the context of a trispecific antigen-binding molecule comprising a first, a second, and a third antigen-binding domain (e.g., a trispecific antibody), the CDRs of the first antigen-binding domain may be designated with the prefix “A1”, the CDRs of the second antigen-binding domain may be designated with the prefix “A2”, and the CDRs of the third antigen-binding domain may be designated with the prefix “A3”. Thus, the CDRs of the first antigen-binding domain may be referred to herein as A1-HCDR1, A1-HCDR2, and A 1-HCDR3; the CDRs of the second antigen-binding domain may be referred to herein as A2-HCDR1, A2-HCDR2, and A2-HCDR3; and the CDRs of the third antigen-binding domain may be referred to herein as A3-HCDR1, A3-HCDR2, and A3-HCDR3.

The bispecific antigen-binding molecules discussed above or herein may be bispecific antibodies. In some cases, the bispecific antibody comprises a human IgG heavy chain constant region. In some cases, the human IgG heavy chain constant region is isotype IgG1. In some cases, the human IgG heavy chain constant region is isotype IgG4. In various embodiments, the bispecific antibody comprises a chimeric hinge that reduces Fc [symbol] receptor binding relative to a wild-type hinge of the same isotype.

In some embodiments, the CD28 multispecific antibody is any one of the multispecific antibodies in Table 3.

TABLE 3
CD28 multispecific antibodies

| Name | Targets |
|---|---|
| REGN7075 | EGFR/CD28 |
| REGN5678 | PSMA/CD28 |
| SAR442257 | CD38/CD28 × CD3 |
| REGN5668 | MUC16/CD28 |
| REGN7945 | CD38/CD28 |
| REGN5837 | CD22/CD28 |

In some embodiments, the target antigen is an antigen associated with the tumor microenvironment of the tumor. As used herein, an antigen associated with the tumor microenvironment includes any antigen on a cell within the stroma, the interstitial fluids surrounding the tumor, blood vessels surrounding the tumor, and the extracellular matrix. Also included are antigens associated with immune cells, other cells, fibroblasts, or signaling molecules in the tumor microenvironment.

In some embodiments, the target antigen is an antigen associated with the tumor stroma selected from PSA, CEA, CA-125, CA-19, COL10, FAP, B7H3, LRRC15, and fibronectin-EDB isoform.

In some embodiments, the target antigen is an antigen associated with the extracellular matrix of the tumor selected from nectin (e.g., nectin-3 or nectin-4), versican (VACN), fibronectin and a carcinoembryonic antigen-related cell adhesion molecules (CEACAM) protein.

In some embodiments, the target antigen is an antigen expressed on the surface of a cancer-associated fibroblast (e.g., α-smooth muscle actin (α-SMA), fibroblast activation protein (FAP), S100A4, platelet-derived growth factor receptors (PDGFRα/β), vimentin, PDPN, CD70, CD10, GPR77, CD10, CD74, CD146, CAV1, Saa3-, or CD49e).

In some embodiments, the target antigen is an antigen expressed on the surface of a blood vessel in the tumor microenvironment, such as DLK1, EphA2, HBB, NG2, NRP1, NRP2, PDGFRβ, PSMA, RGS5, TEM1, VEGFR1 and VEGFR2.

In some embodiments, the target antigen is an immune antigen. In some embodiments, the immune antigen is an antigen expressed on the surface of an immune cell. The immune cell may be a macrophage, a neutrophil, an eosinophil, a basophil, a mast cell, a monocyte, a dendritic cell, a natural killer cell, a T cell or a B cell. In some embodiments, the immune cell has infiltrated the tumor or tumor microenvironment of the tumor. The immune antigen may be selected from any one of the immune antigens listed in Table 4.

TABLE 4
Exemplary Immune Antigens

| Immune Cell Type | Target Antigen |
|---|---|
| macrophages | CD11b/Integrin alpha M |
| | CD14 |
| | CD68 |
| | Fc gamma RIII/CD16, |
| | Fc gamma RI/CD64 |
| | CCR5 |
| B cells | IgM |
| | CD19 |
| | CD80⁺ |
| | CD86⁺ |
| | CD44⁺ |
| | CD69⁺ |
| | PD-L1⁺ |
| | CD27⁺ |
| | CD5⁺ |
| T cells | CD27 |
| | CD28 |
| | CD127 |
| | PD-1 |
| | CD122 |
| | CD132 |
| | KLRG-1 |
| | CD58 |
| | CD99 |
| | CD62L |
| | CD103 |
| | CCR4 |
| | CCR5 |
| | CCR6 |
| | CCR9 |
| | CCR10 |
| | CXCR3 |
| | CXCR4 |
| | CLA |
| | Granzyme A |
| | Granzyme B |
| | Perforin |
| | CD161 |
| | IL-18Ra |
| | c-Kit |
| | CD130 |
| dendritic cells | BDCA-1 |
| | CD8 |
| | CD8alpha |
| | CD11b |
| | CD11c |
| | CD103 |
| | CD205 |
| neutrophils | CD11b |
| | CD16 |
| | CD32 |
| | CD44 |
| | CD55 |
| | CD15 |
| | CD33 |
| | CD45 |
| | CD66b+ |
| | CD18 |
| | CD62L |
| mast cell | CD33 |
| | CD45 |
| | CD117 |
| | CD203c |
| | CD32 |
| | Fc epsilon RO alpha+ |
| | IL-3 R alpha |
| | CD63 |
| | CD203 |
| eosinophil | CD11b+ |
| | CD62L+ |
| | CDR3 |
| | CD11b |
| | CD14 |
| | CD15 |
| | CD16 |
| | CD45 |
| | CD66b |
| | EMR1 |
| | HLA-DR |
| | CD125 |
| | CD49d |
| | Siglec-8 |
| basophil | CCR3 |
| | CD11c |
| | CD22 |
| | CD45 |
| | CD69 |
| | CD117 |
| | CRTH-2 |
| | ENPP-3 |
| | Fc epsilon RI alpha |
| | HLA-DR |
| | IL-3 R alpha |
| | CD49b |
| | CD13 |
| | CD107a |
| | CD164 |
| monocytes | CD14 |
| | CD11b |
| | CCR2 |
| | CD16 |
| | CD141 |
| | CD11c |
| | HLA-DR |
| | CCR7 |
| | CCR5 |
| | CD62L |
| | CX3CR1 |
| | CD68 |
| natural killer cells | KLRK1 |
| | NCR1 |
| | NCR2 |
| | NCR3 |
| | KLRB1 |
| | CD122 |
| | KLRD1 |
| | ITGB2 |
| | KIR receptors |
| | PRF1 |
| | IFNG |
| | CD56 |
| | CD16 |
| | CCR7 |
| | CSF2 |
| | CXCR3 |
| | IL2RB |
| | KLRC1 |
| | SELL |

The first antigen-binding domain and the second antigen-binding domain may be directly or indirectly connected to one another to form a bispecific antigen-binding molecule disclosed herein. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin CH3 domain. A non-limiting example of a multimerizing component is an Fc portion of an immunoglobulin (comprising a CH2-CH3 domain), e.g., an Fc domain of an IgG selected from the isotypes IgG1, IgG2, IgG3, and IgG4, as well as any allotype within each isotype group.

Bispecific antigen-binding molecules disclosed herein will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules disclosed herein. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the methods provided herein include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats).

In the context of bispecific antigen-binding molecules provided herein, the multimerizing domains, e.g., Fc domains, may comprise one or more amino acid changes (e.g., insertions, deletions or substitutions) as compared to the wild-type, naturally occurring version of the Fc domain. For example, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a CH2 or a CH3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

In certain embodiments, provided herein are bispecific antigen-binding molecules comprising a first CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). See, for example, U.S. Pat. No. 8,586,713. Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a CH2 sequence derived from a human IgG1, human IgG2 or human IgG4 CH2 region, and part or all of a CH3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 CH1]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 CH1]-[IgG1 upper hinge]-[IgG2 lower hinge]-[IgG4 CH2]-[IgG1 CH3]. These and other examples of chimeric Fc domains that can be included in any of the antigen-binding molecules disclosed herein are described in US Publication 2014/0243504, published Aug. 28, 2014, which is herein incorporated in its entirety. Chimeric Fc domains having these general structural arrangements, and variants thereof, can have altered Fc receptor binding, which in turn affects Fc effector function.

The CD28 multispecific (e.g., bispecific or trispecific) antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies disclosed herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding (e.g., as measured by cell binding titration or FACS binding) or binding affinity (e.g., KD), improved or enhanced antagonistic or agonistic biological properties (as the case may be), reduced immunogenicity, etc. Antibodies and antigen-binding fragments obtained in this general manner are encompassed within the present disclosure.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the tumor cells in the tumor do not express the target antigen. In some embodiments, less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the tumor cells in the tumor do not express the target antigen. In some embodiments, a percentage range of tumor cells in the tumor do not express the target antigen, wherein the upper and lower percentage range are disclosed herein.

Measurement of expression or level of tumor antigens can be accomplished through any method known in the art, including direct detection of antigen surface expression through flow cytometry. Therefore, as used herein, the phrase “cells in the tumor that do not express the target antigen” may refer to the surface protein expression of the antigen. For example, tumor antigens can be identified by direct interrogation of the tumor immunopeptidome; i.e., all endogenous peptides that are presented by MHC molecules on the cell surface. In this approach, after extraction from tumor cells, peptides are eluted from their complexes with MHC molecules and then subjected to liquid chromatography coupled with tandem mass spectrometry (LC-MS/MS). MS spectra may be compared with customized databases, which are generated by combining sequencing data from patients' tumors with the reference protein sequences. In some embodiments, quantification of tumor antigens m a platform termed SureQuant-IsoMHC, which utilizes a series of pMHC isotopologues and internal standard-triggered targeted mass spectrometry to generate an embedded multipoint calibration curve to determine endogenous pMHC concentrations. For more details see Stopfer L E, Gajadhar A S, Patel B, Gallien S, Frederick D T, Boland G M, Sullivan R J, White F M. Absolute quantification of tumor antigens using embedded MHC-I isotopologue calibrants. Proc Natl Acad Sci USA. 2021 Sep. 14; 118(37), hereby incorporated by reference in it is entirety. Additionally, T cell receptor (TCR)-mimetic antibodies can be used to estimate antigen copy number. Alternatively, measurement of expression of a target antigen (e.g., any target antigen disclosed herein) may be accomplished by measurement of nucleic acid expression (e.g., RNA, such as mRNA expression) in a tumor cell. Nucleic acid expression can be accomplished through nucleic acid amplification and related techniques.

### CD3 Multispecific Antibodies

In some embodiments, the CD28 multispecific antibodies disclosed herein may be administered conjointly with a CD3 antibody (e.g., a CD3 multispecific antibody).

In some embodiments, the CD3 multispecific antibody is any one of the CD3 multispecific antibodies listed in Table 5.

TABLE 5
CD3 multispecific antibodies

| Name | Targets |
|---|---|
| Odronextamab | CD20 × CD3 |
| (REGN1979) | |
| REGN2281 | CD20 × CD3 |
| REGN5458 | BCMA × CD3 |
| REGN1979 | CD20 × CD3 |
| AMV-564 | CD33 × CD3 |
| GBR 1342 | CD38 × CD3 |
| XmAb18968 | CD3 × CD38 |
| SAR443216 | CD3 × CD28 × HER2 |
| A-319 | CD19 × CD3 |
| AMG 330/eluvixtamab | CD33/CD3 |
| emerfetamab | CD33/CD3 |
| ertumaxomab | CD3-HER2neu |
| REGN5459 | BCMA × CD3 |
| REGN4336 | PSMA × CD3 |
| REGN4018 | MUC16 × CD3 |

In some embodiments, the present disclosure includes antibodies having the HCVR, LCVR and/or CDR amino acid sequences of the antibodies set forth herein, the anti-CD3 antibodies disclosed in WO 2014/047231 or WO 2017/053856, the bispecific anti-CD20×anti-CD3 antibodies disclosed in WO 2014/047231, the bispecific anti-PSMA×anti-CD3 antibodies disclosed in WO 2017/023761, the bispecific anti-MUC16×anti-CD3 antibodies disclosed in WO 2018/067331 or WO2018/058003, the bispecific anti-STEAP2×anti-CD3 antibodies disclosed in WO 2018/058001, or the bispecific anti-BCMA×anti-CD3 antibodies disclosed in WO 2020/018820, each of which is incorporated herein by reference.

Additional exemplary CD3 multispecific antibodies that can be used in the compositions and methods disclosed herein include but are not limited to, e.g., bispecific CD3×CD123 antibodies disclosed in U.S. Pat. No. 10,787,521B2, U.S. Patent Application Publication Nos. 2018/0222987A1 and US 2019/0241657A1, and International Application Publication Nos. WO 2016/036937A1, WO 2017/210443A1, WO 2019/050521A1, WO 2019/210147A1, WO 2019/232528A1, and WO 2020/092404A1; bispecific CD3×STEAP2 antibodies disclosed in International Application Publication Nos. WO 2018/058001A1; bispecific CD3×CD20 antibodies disclosed in WO 2014/047231A1, WO 2015/143079A1, WO 2016/081490A1, WO 2017/112775A1, WO 2017/210485A1, WO 2018/114748A1, WO 2018/093821A8, WO 2018/223004A1, WO 2018/188612A1, WO 2019/155008A1, WO 2019/228406A1, WO 2020/088608A1, WO 2020/156405A1, and U.S. Patent Application Publication Nos. US 2020/0199231A1, and US 2020/0172627A1; bispecific CD3×SSTR 2 antibodies disclosed in International Application Publication No. WO 2018/005706A1; bispecific CD3×CD38 antibodies disclosed in International Application Nos. WO 2015/149077A1 and WO 2020/018556A1, and U.S. Patent Application Publication Nos. US 2018/0305465A1 and US 2020/0102403A1; bispecific CD3×STEAP1 antibodies disclosed in Olivier Nolan-Stevaux (2020) Abstract at Proceedings of the Annual Meeting of the American Association for Cancer Research 2020; bispecific CD3×5T4 antibodies disclosed in International Application Publication No. WO 2013/041687A1, U.S. Patent Application Publication Nos. US 2017/0342160A1, US 20200277397A1; bispecific CD3×ENPP3 antibodies as descried in International Application Publication No. WO 2020/180726A1; bispecific CD3×MUC16 antibodies disclosed in International Application Publication Nos. WO 2018/067331A9 and WO 2019/246356A1; bispecific CD3×BCMA antibodies disclosed in International Application Publication Nos. WO 2013/072406A1, WO 2014/140248A1, WO 2016/166629A1, WO 2017/031104A1, WO 2017/134134A1, WO 2017/095267A1, WO 2019/220369A3, WO 2019/075359A1, WO 2019/226761A1, WO 2020/025596A1, WO 2020/191346A1, WO 2020018820A1, U.S. Patent Application Publication Nos. US 2013/0273055A1, US 2019/0263920A1; bispecific CD3×CD19 antibodies disclosed in International Application Publication Nos. WO 2012/055961A1, WO 2016/048938A1, WO 2017/087603A1, WO 2017/096368A1, WO 2018/188612A1, WO 2019/237081A1, WO 2020/048525A1, WO 2020/135335A1, U.S. Patent Application Publication Nos. US 2016/0326249A1, US 2020/0283523A1, US 2019/0284279A1, U.S. Pat. No. 9,315,567B2, U.S. Pat. No. 7,575,923B2, U.S. Pat. No. 7,635,472B2; bispecific CD3×GPRC5D antibodies disclosed in International Application Publication Nos. WO 2018/017786A3, WO 2019/220369A3; bispecific CD3×PSMA antibodies disclosed in U.S. Patent Application Publication No. US 2017/0320947A1; trispecific CD3×CD28×CD38 antibodies disclosed in U.S. Patent Application Publication No. US 2020/0140552A1; or other CD3 multispecific antibodies disclosed in International Application Publication Nos. WO 2016/086189A2, WO 2020/088608A1, WO2019191120A1, and WO 2016/105450A3, the contents of each of which is incorporated by reference herein in its entirety.

In some embodiments, the aforementioned multispecific (e.g., bispecific or trispecific) antigen-binding molecules that specifically bind CD3 and a tumor antigen may comprise an anti-CD3 antigen-binding molecule which binds to CD3 with a weak binding affinity such as exhibiting a KD of greater than about 40 nM, as measured by an in vitro affinity binding assay. The aforementioned bispecific antigen-binding molecules may comprise an anti-CD3 antigen-binding molecule which binds to CD3 and exhibits an EC50 of greater than about 100 nM, as measured by a FACS titration assay. The aforementioned bispecific antigen-binding molecules may comprise an anti-CD3 antigen-binding molecule which exhibits no measurable or observable binding to CD3, as measured by an in vitro affinity binding assay or a FACS titration assay, yet retains ability to activate human PBMC cells and/or induce cytotoxic activity on tumor antigen-expressing cell lines.

### Therapeutic Methods

Provided herein are methods and compositions for mediating killing of a tumor cell in a tumor in a subject by administering to the subject a multispecific antigen binding molecule with a first antigen binding region specific for a target antigen and a second antigen binding region specific for a CD28 protein, wherein the tumor cell does not express or is not predicted to express the target antigen. In some embodiments, at least a subset of tumor cells in the tumor do not express the target antigen.

In some aspects, provided herein are methods and compositions for inducing killing of tumor cells and/or inducing T cell activation against tumor cells in a tumor in a subject, the method comprising administering to the subject a multispecific antigen binding molecule with a first antigen binding region specific for a target antigen and a second antigen binding region specific for a CD28 protein. In some embodiments, at least a subset of tumor cells in the tumor do not express the target antigen.

Also provided herein are methods and compositions for treating cancer in a subject with a tumor, the method comprising administering to the subject a multispecific antigen binding molecule with a first antigen binding region specific for a target antigen and a second antigen binding region specific for a CD28 protein. In some embodiments, at least a subset of tumor cells in the tumor do not express the target antigen.

In some aspects, provided herein are methods of selecting a subject for cancer therapy, the method comprising: i) determining that the subject comprises a tumor comprising tumor cells that do not express a target antigen; and ii) administering to the subject a multispecific antigen binding molecule with a first antigen binding region specific for the target antigen and a second antigen binding region specific for a CD28 protein, optionally wherein the tumor cells do not express the target antigen if the expression of the target antigen is below the level of detection or below signal to noise ratio, thereby selecting a subject for cancer therapy.

In some embodiments, the multispecific antigen binding molecule is a bispecific T cell engager. In some embodiments, the multispecific antigen binding molecule is a bispecific antibody fragment, such as a bispecific T-engaging antibody (BiTE), a dual-affinity re-targeting molecule (DART), or a tandem diabody (TandAb). A bispecific T-cell engager (BiTE) is a small fusion protein containing two antibody binding sites. DARTs are bispecific engagers that use a diabody back bone with the addition of a c-terminal disulfide bridge that improves stabilization. Tandem diabodies (TandAbs) are a type of bispecific antibody fragment. A tandAb is a tetravalent bispecific molecule, 2+2 antigen-binding valency, consisting of Fv domains. TandAbs are typically expressed as a monomeric subunit (single chain Diabody, scDb) with four variable domains from two parental antibodies.

The methods provided herein may further comprise determining that at least the subset (e.g., at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.1%, 30.2%, 30.3%, 30.4%, 30.5%, 30.6%, 30.7%, 30.8%, 30.9%, 31%, 31.1%, 31.2%, 31.3%, 31.4%, 31.5%, 31.6%, 31.7%, 31.8%, 31.9%, 32%, 32.1%, 32.2%, 32.3%, 32.4%, 32.5%, 32.6%, 32.7%, 32.8%, 32.9%, 33%, 33.1%, 33.2%, 33.3%, 33.4%, 33.5%, 33.6%, 33.7%, 33.8%, 33.9%, 34%, 34.1%, 34.2%, 34.3%, 34.4%, 34.5%, 34.6%, 34.7%, 34.8%, 34.9%, 35%, 35.1%, 35.2%, 35.3%, 35.4%, 35.5%, 35.6%, 35.7%, 35.8%, 35.9%, 36%, 36.1%, 36.2%, 36.3%, 36.4%, 36.5%, 36.6%, 36.7%, 36.8%, 36.9%, 37%, 37.1%, 37.2%, 37.3%, 37.4%, 37.5%, 37.6%, 37.7%, 37.8%, 37.9%, 38%, 38.1%, 38.2%, 38.3%, 38.4%, 38.5%, 38.6%, 38.7%, 38.8%, 38.9%, 39%, 39.1%, 39.2%, 39.3%, 39.4%, 39.5%, 39.6%, 39.7%, 39.8%, 39.9%, 40%, 40.1%, 40.2%, 40.3%, 40.4%, 40.5%, 40.6%, 40.7%, 40.8%, 40.9%, 41%, 41.1%, 41.2%, 41.3%, 41.4%, 41.5%, 41.6%, 41.7%, 41.8%, 41.9%, 42%, 42.1%, 42.2%, 42.3%, 42.4%, 42.5%, 42.6%, 42.7%, 42.8%, 42.9%, 43%, 43.1%, 43.2%, 43.3%, 43.4%, 43.5%, 43.6%, 43.7%, 43.8%, 43.9%, 44%, 44.1%, 44.2%, 44.3%, 44.4%, 44.5%, 44.6%, 44.7%, 44.8%, 44.9%, 45%, 45.1%, 45.2%, 45.3%, 45.4%, 45.5%, 45.6%, 45.7%, 45.8%, 45.9%, 46%, 46.1%, 46.2%, 46.3%, 46.4%, 46.5%, 46.6%, 46.7%, 46.8%, 46.9%, 47%, 47.1%, 47.2%, 47.3%, 47.4%, 47.5%, 47.6%, 47.7%, 47.8%, 47.9%, 48%, 48.1%, 48.2%, 48.3%, 48.4%, 48.5%, 48.6%, 48.7%, 48.8%, 48.9%, 49%, 49.1%, 49.2%, 49.3%, 49.4%, 49.5%, 49.6%, 49.7%, 49.8%, 49.9%, 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7%, 55.8%, 55.9%, 56%, 56.1%, 56.2%, 56.3%, 56.4%, 56.5%, 56.6%, 56.7%, 56.8%, 56.9%, 57%, 57.1%, 57.2%, 57.3%, 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5%, 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, 70%, 70.1%, 70.2%, 70.3%, 70.4%, 70.5%, 70.6%, 70.7%, 70.8%, 70.9%, 71%, 71.1%, 71.2%, 71.3%, 71.4%, 71.5%, 71.6%, 71.7%, 71.8%, 71.9%, 72%, 72.1%, 72.2%, 72.3%, 72.4%, 72.5%, 72.6%, 72.7%, 72.8%, 72.9%, 73%, 73.1%, 73.2%, 73.3%, 73.4%, 73.5%, 73.6%, 73.7%, 73.8%, 73.9%, 74%, 74.1%, 74.2%, 74.3%, 74.4%, 74.5%, 74.6%, 74.7%, 74.8%, 74.9%, 75%, 75.1%, 75.2%, 75.3%, 75.4%, 75.5%, 75.6%, 75.7%, 75.8%, 75.9%, 76%, 76.1%, 76.2%, 76.3%, 76.4%, 76.5%, 76.6%, 76.7%, 76.8%, 76.9%, 77%, 77.1%, 77.2%, 77.3%, 77.4%, 77.5%, 77.6%, 77.7%, 77.8%, 77.9%, 78%, 78.1%, 78.2%, 78.3%, 78.4%, 78.5%, 78.6%, 78.7%, 78.8%, 78.9%, 79%, 79.1%, 79.2%, 79.3%, 79.4%, 79.5%, 79.6%, 79.7%, 79.8%, 79.9%, 80%, 80.1%, 80.2%, 80.3%, 80.4%, 80.5%, 80.6%, 80.7%, 80.8%, 80.9%, 81%, 81.1%, 81.2%, 81.3%, 81.4%, 81.5%, 81.6%, 81.7%, 81.8%, 81.9%, 82%, 82.1%, 82.2%, 82.3%, 82.4%, 82.5%, 82.6%, 82.7%, 82.8%, 82.9%, 83%, 83.1%, 83.2%, 83.3%, 83.4%, 83.5%, 83.6%, 83.7%, 83.8%, 83.9%, 84%, 84.1%, 84.2%, 84.3%, 84.4%, 84.5%, 84.6%, 84.7%, 84.8%, 84.9%, 85%, 85.1%, 85.2%, 85.3%, 85.4%, 85.5%, 85.6%, 85.7%, 85.8%, 85.9%, 86%, 86.1%, 86.2%, 86.3%, 86.4%, 86.5%, 86.6%, 86.7%, 86.8%, 86.9%, 87%, 87.1%, 87.2%, 87.3%, 87.4%, 87.5%, 87.6%, 87.7%, 87.8%, 87.9%, 88%, 88.1%, 88.2%, 88.3%, 88.4%, 88.5%, 88.6%, 88.7%, 88.8%, 88.9%, 89%, 89.1%, 89.2%, 89.3%, 89.4%, 89.5%, 89.6%, 89.7%, 89.8%, 89.9%, 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7%, 94.8%, 94.9%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, 100%) of the tumor cells in the tumor or tumor microenvironment do not express the target antigen (e.g., the target antigen, the first target antigen, or the second target antigen).

The methods provided herein may further comprise determining that at least the subset (e.g., at least 0.1%-1%, 1%-5%, 1%-10%, 5%-10%, 10%-15%, 10%-20%, 15%-20%, 15%-25%, 20%-25%, 25%-30%, 20%-30%, 25%-35%, 30%-35%, 30%-40%, 35%-40%, 40%-45%, 40%-50%, 45%-50%, 50%-55%, 50%-60%, 55%-60%, 60%-65%, 60%-70%, 65%-70%, 65%-75%, 70%-75%, 70%-80%, 75%-85%, 75%-80%, 80%-90%, 85%-90%, 85%-95%, 90%-95%, 90%-100%, or 95%-100%) of the tumor cells in the tumor or tumor microenvironment do not express the target antigen (e.g., the target antigen, the first target antigen, or the second target antigen).

It would be appreciated by a person of ordinary skill in the art that the present methods may be utilized with a subject whose tumor does not express the target antigen (e.g., 100% of tumor does not express or is not predicted to express the target antigen) if a portion of the cells in the tumor microenvironment do express or are predicted to express the target antigen.

In some aspects, provided herein are methods of treating cancer in a subject with a tumor, inducing or mediating killing of tumor cells in a tumor in a subject, and/or inducing T cell activation against tumor cells in a tumor in a subject, the method comprising administering to the subject: a first multispecific antigen binding molecule with an antigen binding region specific for a first target antigen and an antigen binding region specific for a CD28 protein; and a second multispecific antigen binding molecule with an antigen binding region specific for a second target antigen and an antigen binding region specific for a CD3 protein, wherein the first target antigen is not the same antigen as the second target antigen. For example, both the first target antigen and the second target antigen can be selected from an antigen listed in Table 2, the first target antigen and the second target antigen cannot be the same antigen.

In some embodiments, the first or second multispecific antigen binding molecule is a bispecific T cell engager. In some embodiments, the first or second multispecific antigen binding molecule is a bispecific antibody fragment, such as a bispecific T-engaging antibody (BiTE), a dual-affinity re-targeting molecule (DART), or a tandem diabody (TandAb).

Thus, in certain embodiments, agents disclosed herein may be used alone or conjointly administered with another type of therapeutic agent. For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

In certain embodiments, provided herein are methods comprising administering to a subject a multispecific antigen binding molecule at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In certain embodiments, provided herein is a composition, e.g., a pharmaceutical composition, containing at least one agent described herein together with a pharmaceutically acceptable carrier. In one embodiment, the composition includes a combination of multiple (e.g., two or more, three or more, four or more, or five or more) agents described herein.

In some embodiments, the pharmaceutical composition is delivered locally or systemically. In some embodiments, the pharmaceutical composition may be administered locally to a tumor present in the subject or the tumor microenvironment. In some embodiments, the agent or pharmaceutical composition is administered with a second cancer therapeutic agent.

The agents described herein may be administered conjointly with any other cancer therapy, including immunotherapies. Additional cancer therapies include immune checkpoint inhibition. In some embodiments, the immune checkpoint inhibitor inhibits an immune checkpoint protein. Immune checkpoint inhibition broadly refers to inhibiting the checkpoints that cancer cells can produce to prevent or downregulate an immune response. Examples of immune checkpoint proteins are CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, A2aR, and combinations thereof. The immune checkpoint inhibitor may be cemiplimab (REGN2810), nivolumab (BMS-936558, MDX-1106, ONO-4538), pembrolizumab (MK-3475, SCH 900475), atezolizumab (MPDL3280A, RG7446, R05541267), durvalumab (MEDI4736, MEDI-4736), avelumab (MSB0010718C), ipilimumab (BMS-734016, IBI310, MDX-010), SHR1210, sintilimab (IBI308), spartalizumab (PDR001), tislelizumab (BGB-A317), pidilizumab, BCD-100, toripalimab (JS001), BAY 1905254, ASP 8374, PF-06801591, AMP-224, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, CX-188, INCB086550, FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, LY3415244, INCB086550, CA-170, CX-072, ADU-1604, AGEN1181, AGEN1884, MK-1308, REGN4659, XmAb22841, ATOR-1015, PSB205, MGD019, AK104, XmAb20717, BMS-986249, tremelimumab, BMS-986258, BGB-A425, INCAGN02390, Sym023, JNJ 61610588, BI 754111, LAG525, MK-4280, REGN3767, Sym022, TSR-033, relatlimab, JTX-2011, MGD009, BMS-986207, OMP-313M32, MK-7684 or TSR-022.

Additional cancer immunotherapies include adoptive immunotherapies such as autologous or allogenic T cell therapy or autologous or allogenic CAR T cell therapy. Adoptive immunotherapy is a treatment method designed to boost a patient's immune response against a tumor or cancer cells. The method involves the removal of immune cells from an individual, the forming of effector cells ex vivo, the expansion of the cells to clinically-relevant numbers and the re-infusion of the cells into the patient. Provided herein are methods that include conjoint administration of an agent disclosed herein and an allogeneic or autologous CTLs expressing a T cell receptor that specifically binds to an peptide (e.g., a cancer peptide or a subject-specific peptide) presented on a class I MHC. In some embodiments, the CTLs are from a cell bank or from the subject to which the CTLs are being administered. In some embodiments, the MHC is a class I MHC. In some embodiment, the class II MHC has an a chain polypeptide that is HLA-DMA, HLA-DOA, HLA-DPA, HLA-DQA or HLA-DRA. In some embodiments, the class II MHC has a β chain polypeptide that is HLA-DMB, HLA-DOB, HLA-DPB, HLA-DQB or HLA-DRB. In some embodiments, the CTLs are stored in a cell library or bank before they are administered to the subject.

The additional cancer therapy may be a cell therapy. As used herein, a cell therapy includes, for example, tumor-infiltrating lymphocytes, modified TCR lymphocytes, or modified CAR lymphocytes. The methods disclosed herein also include T-cell therapy, as well as any other adoptive therapy, such as therapy with natural killer cells or macrophage therapy. The cell therapy may contain unmodified cells, such as in traditional TIL therapy, or genetically modified cells. The methods included herein comprise any method known in the art to achieve the targeting of cells in the cell therapy to tumor targets. As an example, the cell therapy may include cells comprising chimeric antigen receptors (CARs). Single chain antibodies may be used, and CARs may also contain costimulatory domains. CAR cell targets may be on the membrane of the target cells, while TCR modifications may use intracellular targets.

In one embodiment, the cell therapy comprises cells of a type selected from the group consisting of T cells, CD8+ cells, CD4+ cells, NK cells, δ-γ T cells, regulatory T cells, and peripheral mononuclear cells blood. In another embodiment, TILs, T cells, CD8+ cells, CD4+ cells, NK cells, δ-γ T cells, regulatory T cells, or peripheral blood mononuclear cells form cell therapy as disclosed herein. In one specific embodiment, the cell therapy comprises T cells. As used herein, “tumor infiltrating lymphocytes” or TIL refers to white blood cells that have left the bloodstream and migrated into the tumor. Lymphocytes can be divided into three groups containing B cells, T cells, and natural killer cells. In another specific embodiment, the cell therapy comprises T cells that are modified with target specific chimeric antigen receptors or, in particular, selected T cell receptors. As used herein, “T cells” includes, but is not limited to, CD3+ cells, including CD4+ helper cells, CD8+ cytotoxic T cells, and γδ T cells.

In some embodiments, T cells are contacted with antigen presenting cells (APCs) that present a peptide specific to the cancer or tumor in the subject. In some embodiments the APCs are B cells, antigen presenting T-cells, dendritic cells, or artificial antigen-presenting cells (e.g., aK562 cells). Dendritic cells for use in the process may be prepared by taking PBMCs from a patient sample and adhering them to plastic. Generally, the monocyte population sticks and all other cells can be washed off. The adherent population is then differentiated with IL-4 and GM-CSF to produce monocyte derived dendritic cells. These cells may be matured by the addition of IL-1β, IL-6, PGE-1 and TNF-α (which upregulates the important co-stimulatory molecules on the surface of the dendritic cell) and are then transduced with one or more of the peptides provided herein. In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference. Exemplary methods of producing antigen presenting cells can be found in WO2013088114, hereby incorporated in its entirety.

Another exemplary adoptive immunotherapy protocol involves the administration of autologous tumor infiltrating lymphocytes (TIL). TIL cells are potent at killing. TIL cells are effector cells differentiated in vivo in solid tumors (see, U.S. Pat. No. 5,126,132, which describes a method for generating TIL cells for adoptive immunotherapy of cancer). TIL cells may be produced, for example, by removing a tumor sample from a patient, isolating lymphocytes that were infiltrating into 10 the tumor sample, growing these TIL cells ex vivo in the presence of IL-2 and reinfusing the cells to the patient along with IL-2.

The additional cancer therapy may be CAR-T cell therapy. Chimeric antigen receptors (CAR) are molecules combining antibody-based specificity for tumor-associated surface antigens with T cell receptor-activating intracellular domains with specific anti-tumor cellular immune activity (Eshhar, 1997, Cancer Immunol Immunother 45(3-4) 131-136; Eshhar et al., 1993, Proc Natl Acad Sci USA 90(2):720-724; Brocker and Karjalainen, 1998, Adv Immunol 68:257-269). These CARs allow a T cell to achieve MHC-independent primary activation through single chain Fv (scFv) antigen-specific extracellular regions fused to intracellular domains that provide T cell activation and co-stimulatory signals. Second and third generation CARs also provide appropriate co-stimulatory signals via CD28 and/or CD137 (4-1BB) intracellular activation motifs, which augment cytokine secretion and anti-tumor activity in a variety of solid tumor and leukemia models (Pinthus, et al, 2004, J Clin Invest 114(12):1774-1781; Milone, et al., 2009, Mol Ther 17(8):1453-1464; Sadelain, et al., 2009, Curr Opin Immunol 21(2):215-223). Chimeric Antigen Receptor (CAR) T cell therapy involves genetic modification of patient's autologous T-cells to express a CAR specific for a tumor antigen, following by ex vivo cell expansion and re-infusion back to the patient. CARs are fusion proteins of a selected single-chain fragment variable from a specific monoclonal antibody and one or more T cell receptor intracellular signaling domains. This T cell genetic modification may occur either via viral-based gene transfer methods or nonviral methods, such as DNA-based transposons, CRISPR/Cas9 technology or direct transfer of in vitro transcribed-mRNA by electroporation.

The additional cancer therapy may be a natural killer cell therapy. Natural killer (NK) cells can recognize tumor cells as targets and as such may be useful for immunotherapy of cancer (Vivier et al., 2011, Science 331:44-49; Ruggeri et al., 2002, Science 295:2097-2100; Cooley et al., 2010, Blood 116:2411-2419; Miller et al., 2005, Blood 105:3051-3057; Rubnitz et al., 2010, J Clin Oncol. 28:955-959). Infusions of NK cells have been used to treat patients with various forms of cancer (Vivier et al., 2011, Science 331:44-49; Caligiuri, 2008, Blood 112(3):461-469; Ruggeri et al., 2002, Science 295:2097-2100; Miller et al., 2005, Blood 105:3051-3057). Methods are available that make it possible to obtain a large number of human NK cells that demonstrate a higher anti-tumor capacity than that of non-expanded NK cells (see U.S. Pat. No. 7,435,596; Imai et al., 2005, Blood 106:376-83; Fujisaki et al., 2009, Cancer Res. 69: 4010-4017; Cho et al., 2010, Clin Cancer Res. 16:3901-3909). Included herein are NK cells expanded from primary peripheral blood mononucleated cells (PBMCs). Also included herein are NK cells comprising a chimeric antigen receptor or other modifications.

The additional cancer therapy may be macrophage cell therapy. In some embodiments, the cell therapy comprises ex vivo-grown cytotoxic macrophages. Macrophages cells can recognize tumor cells as targets and as such may be useful for immunotherapy of cancer (Andreesen R, Hennemann B, Krause S W. Adoptive immunotherapy of cancer using monocyte-derived macrophages: rationale, current status, and perspectives. J Leukoc Biol. 1998 October; 64(4):419-26). Macrophages are potent immune effector cells whose functional plasticity leads to antitumor as well as protumor function in different settings, and this plasticity has led to notable efforts to deplete or repolarize tumor-associated macrophages. Alternatively, in some embodiments, macrophages are adoptively transferred after, for example, ex vivo genetic modification (Anderson N R, Minutolo N G, Gill S, Klichinsky M. Macrophage-Based Approaches for Cancer Immunotherapy. Cancer Res. 2021 Mar. 1; 81(5):1201-1208).

Also provided herein are methods of treating cancer in a subject by obtaining a sample comprising T-cells from the subject, isolating the cytotoxic T lymphocytes (CTLs) from the sample, expanding the CTLs ex vivo, and administering to the subject the expanded CTLs conjointly with at least one agent (e.g., any agent disclosed herein). The cytotoxic T cells may be tumor-infiltrating lymphocytes. Expanding the CTLs may comprise contacting the CTLs with antigen presenting cells (APCs) expressing a cancer-specific or tumor-specific antigen to create antigen-specific CTLs. In some embodiments, the sample comprising T-cells or the isolated CTLs is irritated prior to administration to the subject. The method may further comprises contacting the CTLs with an anti-CD3 monoclonal antibody (OKT3) prior to administration to the subject. In other embodiments, the method further comprises contacting the CTLs with human interleukin (IL)-2 prior to administration to the subject.

An additional cancer therapy also includes any known stimulating agents of immune cells, such agents include those that induce, for example, proliferation expansion, or activation of such immune cells. Exemplary stimulating methods include administration of stimulatory cytokines, such as IL-2, IL-12, IL-15, IL-18, and IL-21.

In some embodiments, the subject has received a chemotherapy drug prior to administration of the agent. The subject may be refractory to a chemotherapy drug. The subject may receive a chemotherapeutic agent sequentially or simultaneously to receiving an agent of additional cancer therapy disclosed herein. Chemotherapeutic agents include alkylating agents such as thiotepa and cyclophosphamide (Cytoxan™); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide, and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammall and calicheamicin phili); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (Taxol™, Bristol Meyers Squibb Oncology, Princeton, N.J.) and docetaxel (Taxoteret™, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™) raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprohde, and goserelin; and pharmaceutically acceptable salts, acids or derivatives of any of the above.

As described in detail below, the pharmaceutical compositions and/or agents disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation. Methods of preparing pharmaceutical formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

The pharmaceutical compositions provided herein can be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding molecule administered to a patient may vary depending upon the age and the size of the patient, target disease, conditions, route of administration, and the like.

Various delivery systems are known and can be used to administer a pharmaceutical composition provided herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In some embodiments, a pharmaceutical composition provided herein can be delivered subcutaneously or intravenously with a standard needle and syringe. In addition, with respect to subcutaneous delivery, a pen delivery device readily has applications in delivering a pharmaceutical composition disclosed herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition disclosed herein. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition disclosed herein include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The agents described herein may be administered in multiple doses, such as initial doses, secondary doses and tertiary doses. The terms “initial dose,” “secondary doses,” and “tertiary doses,” refer to the temporal sequence of administration of the antigen-binding molecule disclosed herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the therapeutic agents described herein, but generally may differ from one another in terms of frequency of administration. In certain embodiments, however, the amount of an antigen-binding molecule contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment disclosed herein, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of the therapeutic agents described herein which is administered to a patient prior to the administration of the very next dose in the sequence with no intervening doses.

The methods according to this aspect disclosed herein may comprise administering to a patient any number of secondary and/or tertiary doses of the therapeutic agents described herein. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Indications

In some embodiments, the methods described herein may be used to treat any cancer, including any cancerous or pre-cancerous tumor. Cancers that may be treated by methods and compositions provided herein include, but are not limited to, cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchioloalveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The compositions or methods are useful for treating a CD20-expressing cancer or tumor. The compositions or methods are useful for treating a B cell malignancy, including non-Hodgkin lymphoma, Hodgkin lymphoma, chronic lymphocytic leukemia, acute lymphoblastic leukemia, small lymphocytic lymphoma, diffuse large B-cell lymphoma, follicular lymphoma, mantle cell lymphoma, marginal zone lymphoma, Waldenstrom macroglobulinemia, primary mediastinal B-cell lymphoma, lymphoblastic lymphoma, or Burkitt lymphoma. In some embodiments, the cancer is follicular lymphoma. In some embodiments, the cancer is diffuse large B-cell lymphoma (DLBCL).

In some embodiments, the cancer comprises a solid tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

In certain embodiments, the tumor being treated is one that is not expected to express a particular target antigen. Expression profiles for exemplary target antigens (e.g., exemplary TAAs) disclosed herein are known in the art and/or can be determined using methods provided herein.

Expression profiles are known and can be found, for example, in the following databases: TANTIGEN 2.0 (found on the World Wide Web at projects.met-hilab.org/tadb/index.php; more details can be found in Zhang, G., Chitkushev, L., Olsen, L. R. et al. TANTIGEN 2.0: a knowledge base of tumor T cell antigens and epitopes. BMC Bioinformatics 22, 40 (2021)); the Cancer Epitope Database and Analysis Resource (CEDAR)(more details can be found in Koşloğlu-Yalçin, Z. et al., The Cancer Epitope Database and Analysis Resource: A Blueprint for the Establishment of a New Bioinformatics Resource for Use by the Cancer Immunology Community, Frontiers in Immunology, 12 (2021)); the Human Protein Atlas (found on the World Wide Web at v17.proteinatlas.org/); dbPepNeo (found on the World Wide Web at biostatistics.online/dbPepNeo/search.php); and cBioPortal (found on the World Wide Web at cbioportal.org).

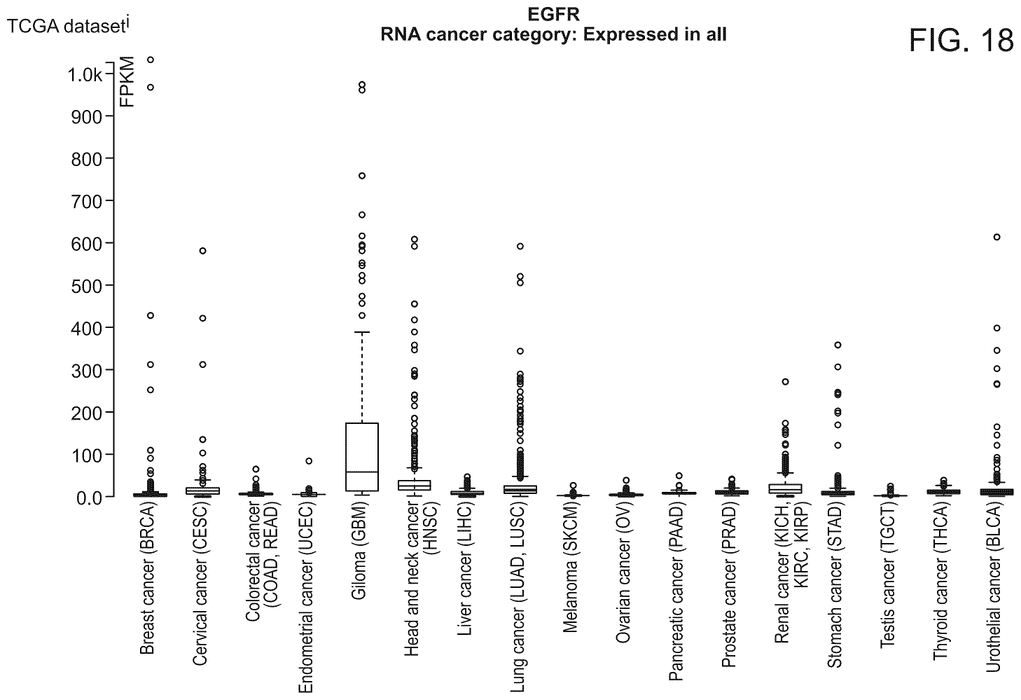

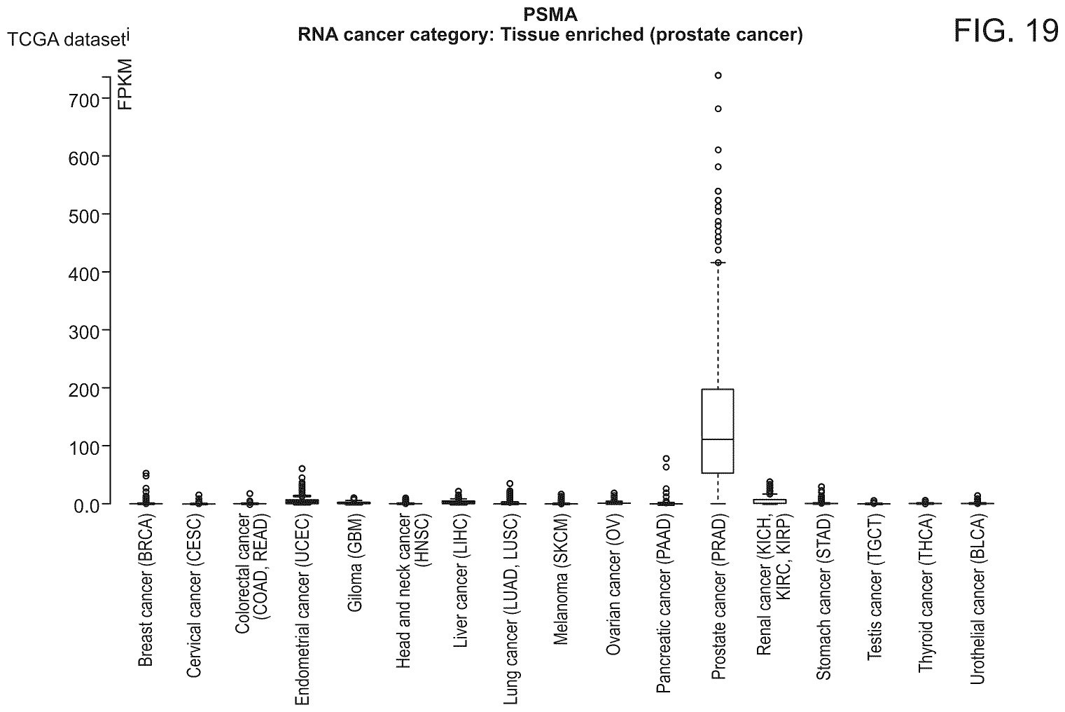

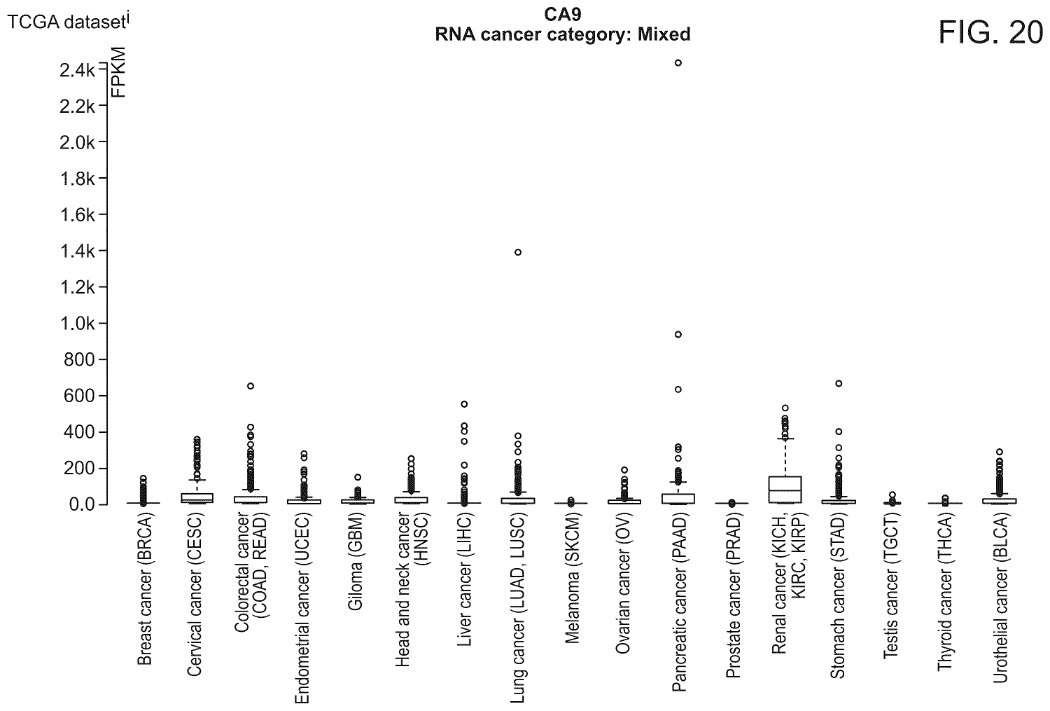

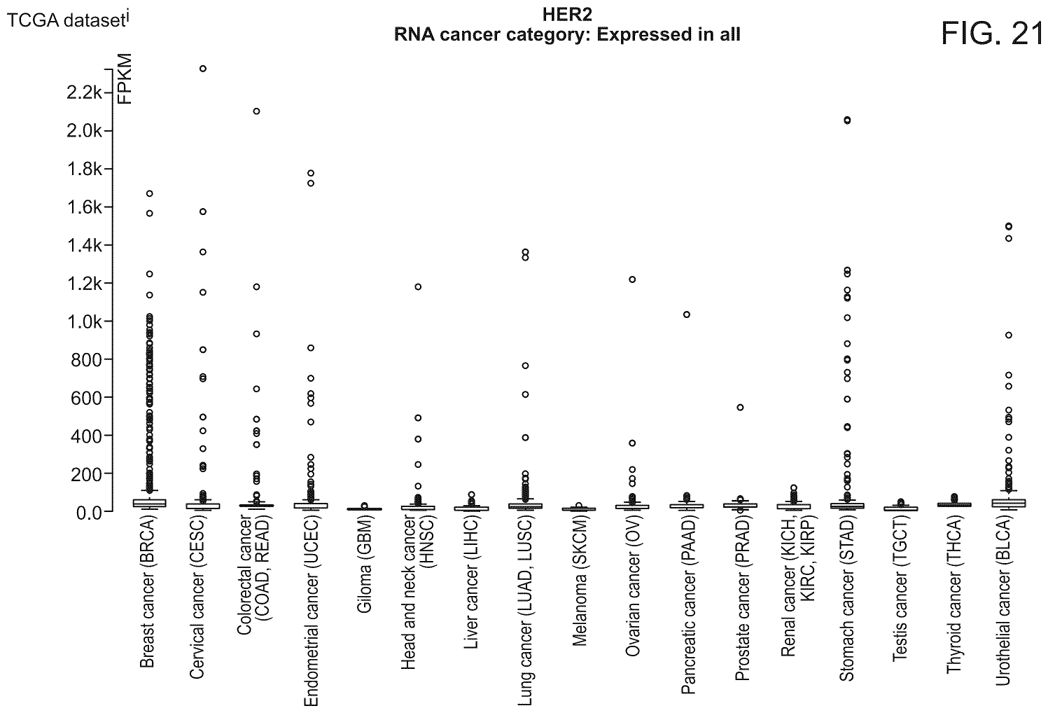

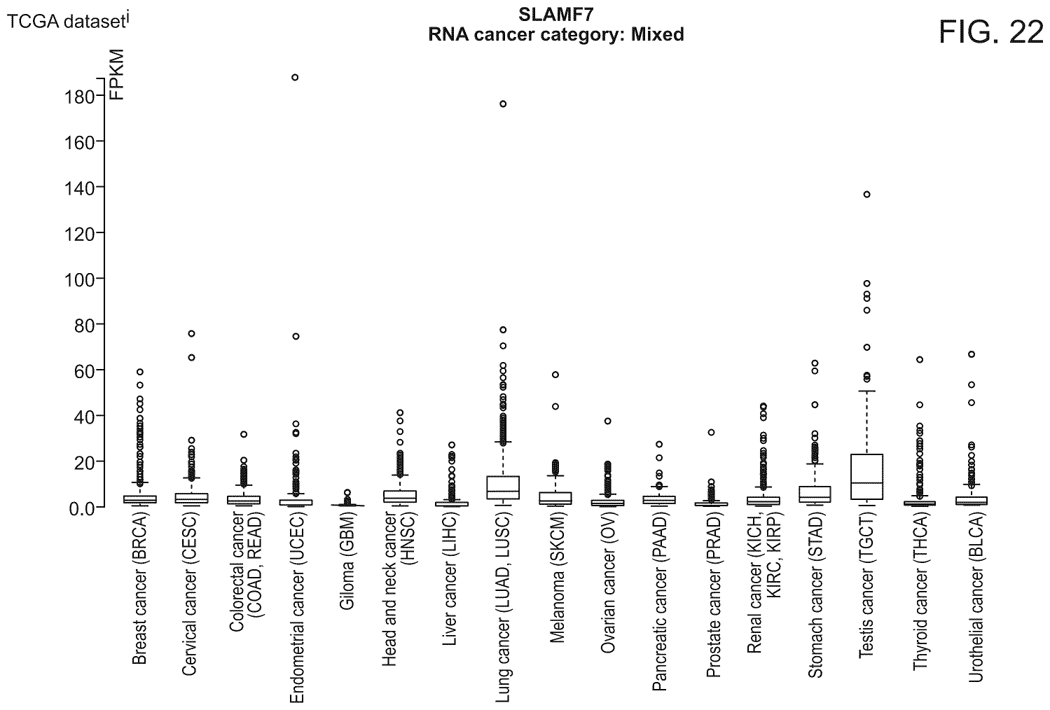

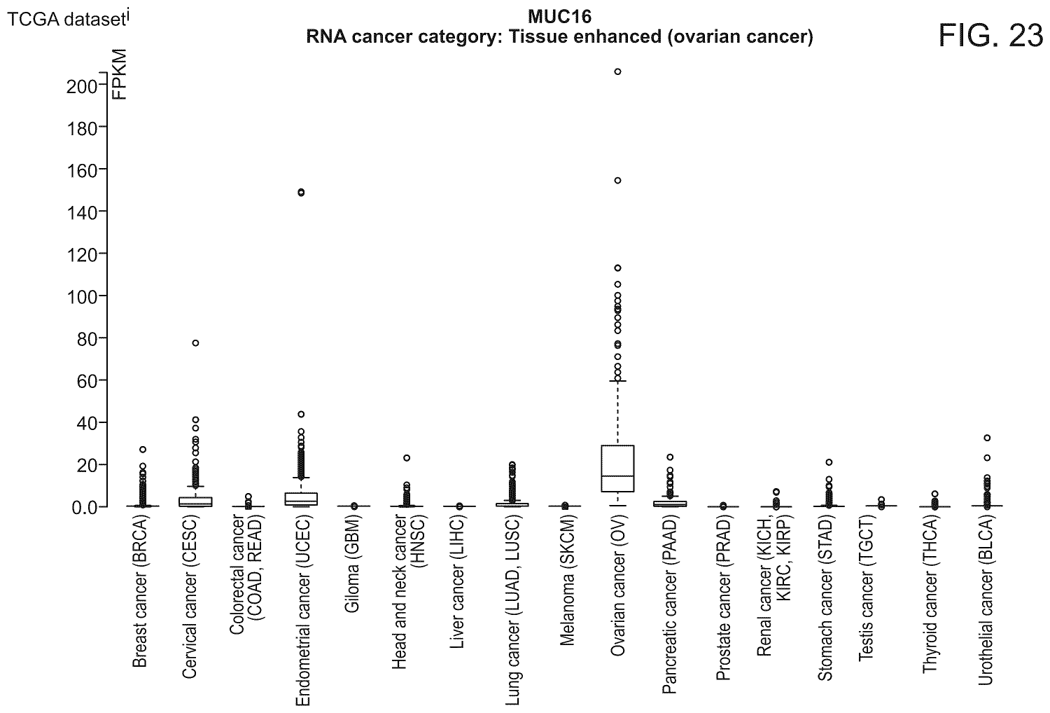

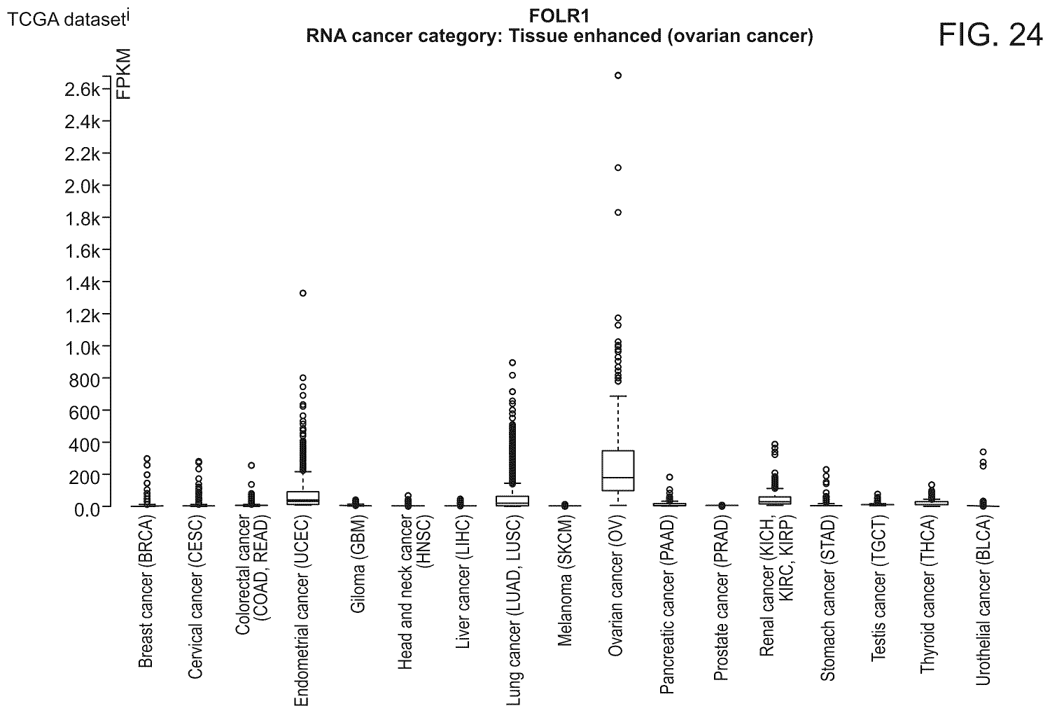

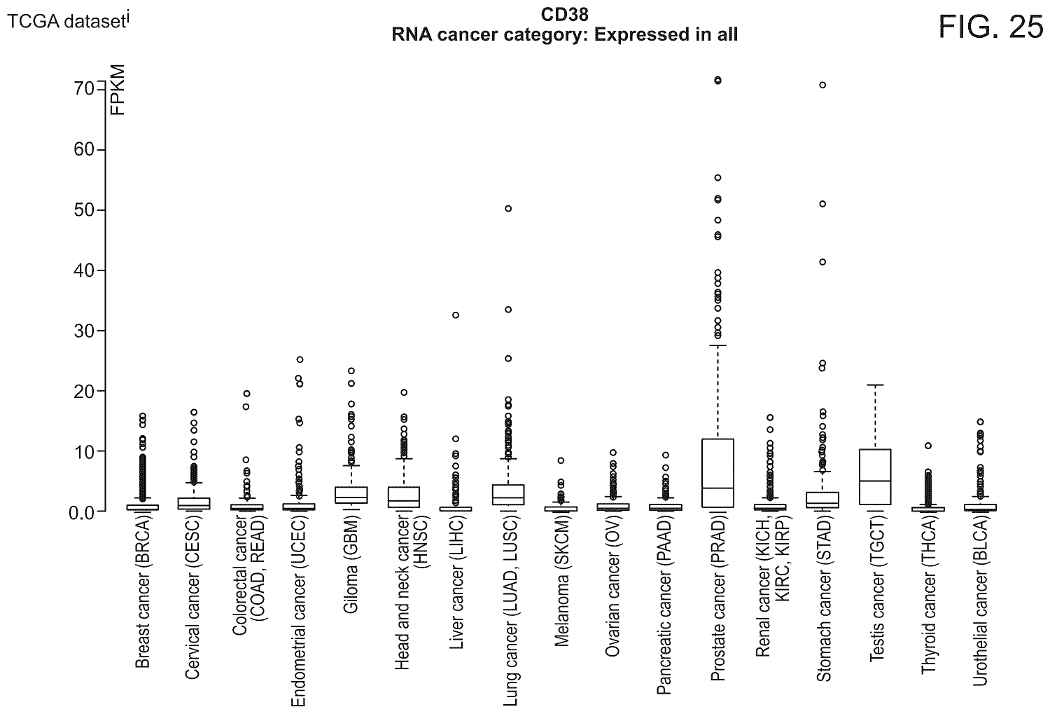

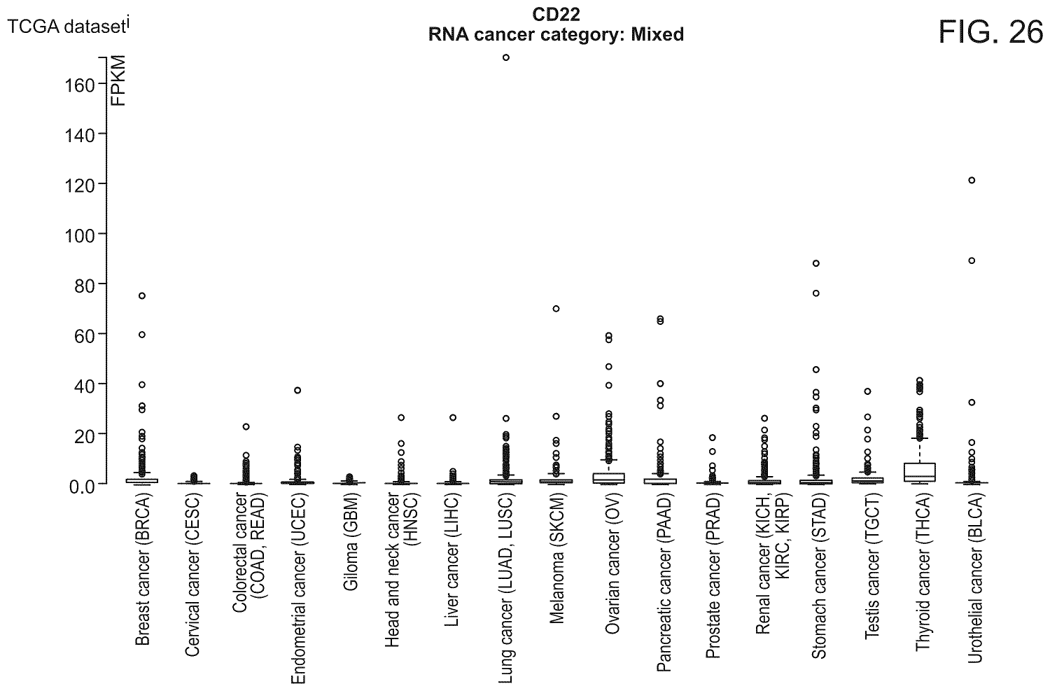

Exemplary expression profiles for certain exemplary target antigens are provided in FIGS. 18-26.

In some embodiments, the tumor is not predicted to express the target antigen based on known expression profiles (e.g., expression profiles disclosed herein). In some embodiments, a tumor type is predicted not to express a target antigen if less than 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, or 50% of analyzed tumors express the target antigen.

Primary tumors exhibit antigen expression patterns that vary intratumorally as well as across patients. The variability in antigen expression between patients harboring the same histologic tumor type can be substantial. Therefore, the exemplary expression profiles disclosed herein are informative as to the tumor types that can be treated using the methods disclosed herein, but will not be limiting to the cancer or tumor type that can be treated using the methods disclosed herein. In some embodiments, the tumor being treated may be a tumor that has been tested and does not express or contains a subset of tumor cells that do not express the target antigen, despite tumor antigen expression profiles showing the tumor type typically expresses the target antigen. In some embodiments, the target antigen is enriched in a specific tumor type, but the target antigen is not expressed in a subset of tumor cells in the specific tumor type in the patient. In some embodiments, the target antigen is typically expressed in a variety of tumor types, but the target antigen is not expressed in a subset of tumor cells in the patient.

### Screening, Diagnostic, and Prognostic Assays

Screening, diagnostic, and/or prognostic assays are also provided for identifying the levels or quantifying the amount of a tumor associated antigen on the cancer or tumor cells in a cancer in a subject.

In some embodiments, at least a subset of tumor cells in the tumor do not express the target antigen. The methods provided herein may further comprise determining or quantifying if at least the subset (e.g., at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%, 17.5%, 17.6%, 17.7%, 17.8%, 17.9%, 18%, 18.1%, 18.2%, 18.3%, 18.4%, 18.5%, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.1%, 30.2%, 30.3%, 30.4%, 30.5%, 30.6%, 30.7%, 30.8%, 30.9%, 31%, 31.1%, 31.2%, 31.3%, 31.4%, 31.5%, 31.6%, 31.7%, 31.8%, 31.9%, 32%, 32.1%, 32.2%, 32.3%, 32.4%, 32.5%, 32.6%, 32.7%, 32.8%, 32.9%, 33%, 33.1%, 33.2%, 33.3%, 33.4%, 33.5%, 33.6%, 33.7%, 33.8%, 33.9%, 34%, 34.1%, 34.2%, 34.3%, 34.4%, 34.5%, 34.6%, 34.7%, 34.8%, 34.9%, 35%, 35.1%, 35.2%, 35.3%, 35.4%, 35.5%, 35.6%, 35.7%, 35.8%, 35.9%, 36%, 36.1%, 36.2%, 36.3%, 36.4%, 36.5%, 36.6%, 36.7%, 36.8%, 36.9%, 37%, 37.1%, 37.2%, 37.3%, 37.4%, 37.5%, 37.6%, 37.7%, 37.8%, 37.9%, 38%, 38.1%, 38.2%, 38.3%, 38.4%, 38.5%, 38.6%, 38.7%, 38.8%, 38.9%, 39%, 39.1%, 39.2%, 39.3%, 39.4%, 39.5%, 39.6%, 39.7%, 39.8%, 39.9%, 40%, 40.1%, 40.2%, 40.3%, 40.4%, 40.5%, 40.6%, 40.7%, 40.8%, 40.9%, 41%, 41.1%, 41.2%, 41.3%, 41.4%, 41.5%, 41.6%, 41.7%, 41.8%, 41.9%, 42%, 42.1%, 42.2%, 42.3%, 42.4%, 42.5%, 42.6%, 42.7%, 42.8%, 42.9%, 43%, 43.1%, 43.2%, 43.3%, 43.4%, 43.5%, 43.6%, 43.7%, 43.8%, 43.9%, 44%, 44.1%, 44.2%, 44.3%, 44.4%, 44.5%, 44.6%, 44.7%, 44.8%, 44.9%, 45%, 45.1%, 45.2%, 45.3%, 45.4%, 45.5%, 45.6%, 45.7%, 45.8%, 45.9%, 46%, 46.1%, 46.2%, 46.3%, 46.4%, 46.5%, 46.6%, 46.7%, 46.8%, 46.9%, 47%, 47.1%, 47.2%, 47.3%, 47.4%, 47.5%, 47.6%, 47.7%, 47.8%, 47.9%, 48%, 48.1%, 48.2%, 48.3%, 48.4%, 48.5%, 48.6%, 48.7%, 48.8%, 48.9%, 49%, 49.1%, 49.2%, 49.3%, 49.4%, 49.5%, 49.6%, 49.7%, 49.8%, 49.9%, 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7%, 55.8%, 55.9%, 56%, 56.1%, 56.2%, 56.3%, 56.4%, 56.5%, 56.6%, 56.7%, 56.8%, 56.9%, 57%, 57.1%, 57.2%, 57.3%, 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5%, 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, 70%, 70.1%, 70.2%, 70.3%, 70.4%, 70.5%, 70.6%, 70.7%, 70.8%, 70.9%, 71%, 71.1%, 71.2%, 71.3%, 71.4%, 71.5%, 71.6%, 71.7%, 71.8%, 71.9%, 72%, 72.1%, 72.2%, 72.3%, 72.4%, 72.5%, 72.6%, 72.7%, 72.8%, 72.9%, 73%, 73.1%, 73.2%, 73.3%, 73.4%, 73.5%, 73.6%, 73.7%, 73.8%, 73.9%, 74%, 74.1%, 74.2%, 74.3%, 74.4%, 74.5%, 74.6%, 74.7%, 74.8%, 74.9%, 75%, 75.1%, 75.2%, 75.3%, 75.4%, 75.5%, 75.6%, 75.7%, 75.8%, 75.9%, 76%, 76.1%, 76.2%, 76.3%, 76.4%, 76.5%, 76.6%, 76.7%, 76.8%, 76.9%, 77%, 77.1%, 77.2%, 77.3%, 77.4%, 77.5%, 77.6%, 77.7%, 77.8%, 77.9%, 78%, 78.1%, 78.2%, 78.3%, 78.4%, 78.5%, 78.6%, 78.7%, 78.8%, 78.9%, 79%, 79.1%, 79.2%, 79.3%, 79.4%, 79.5%, 79.6%, 79.7%, 79.8%, 79.9%, 80%, 80.1%, 80.2%, 80.3%, 80.4%, 80.5%, 80.6%, 80.7%, 80.8%, 80.9%, 81%, 81.1%, 81.2%, 81.3%, 81.4%, 81.5%, 81.6%, 81.7%, 81.8%, 81.9%, 82%, 82.1%, 82.2%, 82.3%, 82.4%, 82.5%, 82.6%, 82.7%, 82.8%, 82.9%, 83%, 83.1%, 83.2%, 83.3%, 83.4%, 83.5%, 83.6%, 83.7%, 83.8%, 83.9%, 84%, 84.1%, 84.2%, 84.3%, 84.4%, 84.5%, 84.6%, 84.7%, 84.8%, 84.9%, 85%, 85.1%, 85.2%, 85.3%, 85.4%, 85.5%, 85.6%, 85.7%, 85.8%, 85.9%, 86%, 86.1%, 86.2%, 86.3%, 86.4%, 86.5%, 86.6%, 86.7%, 86.8%, 86.9%, 87%, 87.1%, 87.2%, 87.3%, 87.4%, 87.5%, 87.6%, 87.7%, 87.8%, 87.9%, 88%, 88.1%, 88.2%, 88.3%, 88.4%, 88.5%, 88.6%, 88.7%, 88.8%, 88.9%, 89%, 89.1%, 89.2%, 89.3%, 89.4%, 89.5%, 89.6%, 89.7%, 89.8%, 89.9%, 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7%, 94.8%, 94.9%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, 100%) of the tumor cells in the tumor or tumor microenvironment do not express the target antigen, and if the determined subset/percentage does not express the target antigen, administering an agent or agents disclosed herein the subject. It would be appreciated by a person of ordinary skill in the art that the present methods may be utilized with a subject whose tumor does not express the target antigen (e.g., about 100% of tumor does not express or is not predicted to express the target antigen) if a portion of the cells in the tumor microenvironment do express or are predicted to express the target antigen.

The methods provided herein may further comprise determining that at least the subset of the tumor cells in the tumor do not express the target antigen. In some embodiments, the tumor cells do not express the target antigen if the expression of the target antigen is below the level of detection or below signal to noise ratio.

Provided herein are methods of screening subjects by measuring or calculating the amount or level of a tumor associated antigen in a subject's cancer. A tumor associated antigen may be measured by any method know in the art. For example, a biological sample may be taken from a patient. Samples may be obtained by any means known in the art. Samples may also be taken directly from the cancer, the tumor, or tumor microenvironment. The term “biological sample” is intended to include tissues, cells, and biological fluids isolated from a subject, as well as tissues, cells, and fluids present within a subject.

In some embodiments, the determined percentage of tumor cells that express a target antigen can be determined by measuring the level or expression of a target antigen from a biological sample (e.g., a biopsy of the tumor or tumor microenvironment) taken from a subject. In some embodiments, said percentage can then be extrapolated to quantify a predicted total percentage of tumor and/or tumor microenvironment cells in the subject that express the target antigen.

A detection method encompassed by the present disclosure may be used to detect mRNA, protein, or genomic DNA of the tumor associated antigen or a biologically active fragment thereof in a biological sample in vitro, as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody against the desired protein to be detected. For example, the antibody may be labeled with a radioactive marker whose presence and location in a subject may be detected by standard imaging techniques.

The assays described herein may include measuring tumor associated antigen levels post isolation from cells (e.g., after a biopsy or isolation of a biological sample). These may be conducted in cell-free formats using known components of gene expression of the tumor associated antigen. It may be desirable to immobilize certain components of the assay and such embodiments may benefit from the use of well-known adaptations for biomolecule immobilization, such as the use of microtitre plates, beads, test tubes, micro-centrifuge tubes in combination with derivatizable moieties, such as fusion protein domains, biotinylation, antibodies, and the like.

To determine whether a subject is afflicted with a condition disclosed herein, has a risk of developing such a condition, or could benefit from administration of the agents disclosed herein, a biological sample may be obtained from a subject and the biological sample may be contacted with a compound or an agent capable of detecting an tumor associated antigen protein or a polynucleotide (e.g., mRNA or genomic DNA) encoding said tumor associated antigen in the biological sample. An agent for detecting the mRNA or genomic DNA may comprise a labeled nucleic acid probe capable of hybridizing to the mRNA or genomic DNA. The nucleic acid probe may be, for example, a sequence that is complementary to the nucleic acid encoding the tumor associated antigen, or a portion thereof, such as an oligonucleotide of at least 15, 20, 25, 30, 25, 40, 45, 50, 100, 250 or 500 nucleotides in length and sufficient to specifically hybridize under stringent conditions to the desired mRNA or genomic DNA. Other suitable probes for use in diagnostic assays encompassed by the present disclosure are described herein.

In another embodiment, the methods further involve obtaining a control biological sample from a control subject, contacting the control sample with a compound or agent capable of detecting protein, mRNA or genomic DNA, such that the presence of the desired protein, mRNA or genomic DNA is detected in the biological sample, and comparing the presence of the protein, mRNA or genomic DNA in the control sample with the presence of the protein, mRNA or genomic DNA in the control sample.

In some embodiments, assays described herein may be conducted in cell-free formats using known components of gene expression of the tumor associated antigen. It may be desirable to immobilize certain components of the assay, and such embodiments may benefit from the use of well-known adaptations for biomolecule immobilization, such as the use of microtitre plates, beads, test tubes, micro-centrifuge tubes in combination with derivatizable moieties, such as fusion protein domains, biotinylation, antibodies, and the like.

Analysis of one or more regions of nucleic acids of a tumor associated antigen in a subject may be useful for predicting whether a subject has or is likely to benefit from the methods disclosed herein. For example, detecting the tumor associated antigen in a portion of the cancer, such that the cancer would be classified as a heterologous cancer, would indicate the subject would benefit from the methods disclosed herein. Similarly, analysis of genomic copy number of a tumor associated antigen in a subject may be useful for predicting whether a subject would benefit from the methods disclosed herein. In some embodiments, methods encompassed by the present disclosure may be characterized as comprising detecting, in a sample of cells from the subject, the presence or absence of one or more polymorphic regions of the gene encoding the tumor associated antigen.

In other detection methods, it is necessary to first amplify at least a portion of nucleic acids detected in the biological sample. Amplification may be performed, e.g., by PCR and/or LCR (see Wu and Wallace, (1989) Genomics 4:560), according to methods known in the art. In one embodiment, genomic DNA of a cell is exposed to two PCR primers and amplification for a number of cycles sufficient to produce the required amount of amplified DNA.

Alternative amplification methods include: self-sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well-known to those of skill in the art. These detection schemes are especially useful for the detection of nucleic acid molecules if such molecules are present in very low numbers.

In one embodiment, any of a variety of sequencing reactions known in the art may be used to directly sequence the nucleic acids in the biological sample, by comparing the sequence of the sample sequence with the corresponding reference (control) sequence. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, may be carried out.

Antibodies directed against the tumor associated antigen may also be used in disease diagnostics and prognostics. In addition, such diagnostic methods, may be used to detect abnormalities in the level of such polypeptide expression, or abnormalities in the structure and/or tissue, cellular, or subcellular location of such polypeptides. Structural differences may include, for example, differences in the size, electronegativity, or antigenicity of the mutant polypeptide relative to the normal polypeptide. Protein from the tissue or cell type to be analyzed may easily be detected or isolated using techniques that are well-known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example (Harlow, E. and Lane, D., 1988, “Antibodies: A Laboratory Manual”, Cold Spring Harbor Laboratory Press, Cold Spring Harbor, New York), which is incorporated herein by reference in its entirety.

This may be accomplished, for example, by immunofluorescence techniques employing a fluorescently labeled antibody (see below) coupled with light microscopic, flow cytometric, or fluorimetric detection. The antibodies (or fragments thereof) useful according to the present disclosure may, additionally, be employed histologically, as in immunofluorescence or immunoelectron microscopy, for in situ detection of the tumor associated antigen. In situ detection may be accomplished by removing a histological specimen from a subject, and applying thereto a labeled antibody disclosed herein. The antibody (or fragment) is may be applied by overlaying the labeled antibody (or fragment) onto a biological sample. Through the use of such a procedure, it is possible to determine not only the presence of a tumor associated antigen, but also its distribution in the examined tissue. One of ordinary skill will readily perceive that any of a wide variety of histological methods (such as staining procedures) may be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier may be either soluble to some extent or insoluble for the purposes encompassed by the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Supports include, but are not limited to, polystyrene beads. Those skilled in the art will know many other suitable carriers for binding antibody or antigen, or will be able to ascertain the same by use of routine experimentation.

One means for labeling an antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, MD; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, F L, 1980; Ishikawa, et al., (eds.) Enzyme Immunoassay, Kgaku Shoin, Tokyo, 1981). The enzyme, which is bound to the antibody, will react with an appropriate substrate, such as a chromogenic substrate, in such a manner as to produce a chemical moiety that may be detected, for example, by spectrophotometric, fluorimetric or by visual means. Enzymes that may be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection may be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope may be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody may also be detectably labeled using fluorescence emitting metals such as 152Eu, or others of the lanthanide series. These metals may be attached to the antibody using such metal chelating groups as diethylenetriaminepentacetic acid (DTPA) or ethylenediaminetetraacetic acid (EDTA).

The antibody also may be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody encompassed by the present disclosure. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific allelic variant of interest. Sample nucleic acid to be analyzed by any of the above-described diagnostic and prognostic methods may be obtained from any cell type or tissue of a subject. For example, a subject's bodily fluid (e.g. blood) may be obtained by known techniques (e.g., venipuncture). Alternatively, nucleic acid tests may be performed on dry samples (e.g., hair or skin). Fetal nucleic acid samples may be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods that focus primarily on the detection of one nucleic acid sequence, profiles may also be assessed in such detection schemes. Fingerprint profiles may be generated, for example, by utilizing a differential display procedure, Northern analysis and/or RT-PCR.

Also provided herein are methods of predicting if the tumor cells in the subject express a target antigen by tissue or tumor expression profiles of the target antigen. For example, the subject may be diagnosed with a cancer, and the tumor cells in the tumor in the subject are not predicted to express the target antigen if known issue or tumor expression profiles typically do not express the target antigen. Additionally, the subject may be diagnosed with a cancer, and the tumor cells in the tumor in the subject are not predicted to express the target antigen if known issue or tumor expression profiles show that less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or less than 75% do not express the target antigen.

The present disclosure further pertains to novel agents identified by the above-described screening assays. Accordingly, screening, diagnostic, and/or prognostic assays are also provided for identifying an antibody specific for a tumor associated antigen that induces/mitigates killing of or activates T cells against tumor cells that do not express the tumor associated antigen.

In one embodiment, the present disclosure provides assays for screening candidate or test compounds which are substrates of or interact with a tumor associated antigen.

In one embodiment, an assay is a cell-based assay in which a cell, such as a cancer cell, is contacted with a test agent, and the ability of the test compound to kill tumor cells or activate T cells is determined. Determining the ability of the test agent to perform the functions discussed may be accomplished by monitoring biomarkers described herein, for example, biopsy, biomarker expression, physical assays, and the like.

The ability of the test agent to modulate the binding of an antibody to its target substrate may also be determined. Determining the ability of the test agent to bind may be accomplished, for example, by coupling the substrate with a radioisotope or enzymatic label such that binding of the substrate to the antibody may be determined by detecting the labeled substrate in a complex. The target substrate may also be coupled with a radioisotope or enzymatic label to monitor the ability of a test agent to modulate binding to the substrate in a complex. Determining the ability of the test agent to bind a target protein may be accomplished, for example, by coupling the agent with a radioisotope or enzymatic label such that binding may be determined by detecting the labeled agent in a complex. For example, such agents may be labeled with ¹²⁵I, ³⁵S, ¹⁴C, or ³H, either directly or indirectly, and the radioisotope detected by direct counting of radioemmission or by scintillation counting. Agents can further be enzymatically labeled with, for example, horseradish peroxidase, alkaline phosphatase, or luciferase, and the enzymatic label detected by determination of conversion of an appropriate substrate to product.

In certain embodiments, the ability of an agent to bind is determined with or without the labeling of any of the interactants. For example, a microphysiometer may be used to detect the interaction without labeling any component (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS).

In one aspect, cell-based systems, as described herein, may be used to identify agents that treat a cancer disclosed herein. For example, such cell systems may be exposed to an agent at a sufficient concentration and for a time sufficient to elicit such an amelioration of disease symptoms in the exposed cells. After exposure, the cells may be examined to determine whether one or more of the disease phenotypes has been altered to resemble a more normal or more wild type phenotype.

In addition, animals or animal-based disease systems, such as those described herein, may be used to identify such agents. Such animal models may be used as test substrates for the identification of drugs, pharmaceuticals, therapies, and interventions which may be effective in binding a tumor associated antigen or other target herein, such as to treat or prevent a cancer disclosed herein.

### EXEMPLIFICATION

### Example 1: CD22-Targeted CD28 Bispecific Antibody Enhances Anti-Tumor Efficacy of Odronextamab in Refractory Diffuse Large B-Cell Lymphoma Model

While many patients with diffuse large B-cell lymphoma (DLBCL) may achieve a complete response to frontline chemoimmunotherapy, patients with relapsed/refractory (r/r) disease typically have poor outcomes. Odronextamab, a CD20×CD3 bispecific antibody that provides “signal 1” through the activation of the T cell receptor/CD3 complex, has exhibited early, promising activity for highly refractory DLBCL patients in Phase I trials; however, not all patients achieve complete responses, and many will relapse, representing a high unmet medical need. Here, it is investigated whether adding a costimulatory “signal 2” by engaging CD28 receptors on T cells could augment Odronextamab activity. The data disclosed herein demonstrate for the first time that REGN5837, a bispecific antibody that crosslinks CD22 expressing tumor cells with CD28 expressing T cells, enhances the activity of Odronextamab by potentiating T cell activation and cytolytic function. In preclinical DLBCL tumor studies using human immune system reconstituted animals, REGN5837 promotes the anti-tumor activity of Odronextamab and induces the intratumoral expansion of plastic reprogrammable T cells while skewing away from a dysfunctional state. REGN5837 monotherapy shows activity and no toxicity in primate studies, and it augments T cell activation when dosed in combination with Odronextamab. In addition, analysis of non-Hodgkin lymphoma (NHL) clinical samples reveals an increase in CD28⁺CD8⁺ T cells post Odronextamab treatment, demonstrating the presence of a population that could be targeted by a CD22×CD28 antibody. Collectively, these data demonstrate that REGN5837 enhance the anti-tumor activity of Odronextamab in preclinical NHL models and the combination of these two bispecific antibodies may provide a novel chemotherapy-free approach for the treatment of r/r DLBCL.

A therapeutic strategy in the field of cancer immunotherapy is the use of bispecific antibodies to redirect T cells to tumors in order to enhance anti-tumor activity. This has led to the approval of Blincyto, a CD19×CD3 T-cell engager for the treatment of acute lymphoblastic leukemia and Removab, an EpCAM×CD3 bispecific antibody for the treatment of malignant ascites. These T cell redirecting therapeutics are designed to engage a tumor antigen with one antigen-binding fragment (Fab) arm and a T cell activating receptor with the other Fab arm. Odronextamab is a CD20×CD3 bispecific antibody that efficiently triggers T cell mediated killing of CD20 expressing cells in preclinical tumor models. Clinical data from a Phase 1 trial indicate promising activity with manageable safety in highly refractory and relapsed patients with B-NHL. However, since heavily pretreated patients invariably relapse, there is still potential to improve efficacy for patients with aggressive lymphomas. For optimal T cell activation, T cells require the engagement of the T cell receptor complex (TCR) which provides “signal 1” and the additional engagement of a costimulatory receptor which provides “signal 2”. While Odronextamab activates T cells by providing signal 1 through the cross-linking of CD3, further enhancement of T cell effector function and activation can be mediated by the addition of a costimulatory signal. In this study, it is examine whether the addition of a CD22×CD28 bispecific antibody can enhance the anti-tumor activity of Odronextamab.

REGN5837, or CD22×CD28, is a hinge-stabilized human IgG4-based antibody designed to enhance T cell responses against CD22 expressing tumors (i.e. NHL) by bridging CD22 expressing cells with CD28 expressing T cells. CD22 is a transmembrane protein that binds sialic acid and is found on normal B cells and malignant B cells such as DLBCL tumors. Although the precise role of human CD22 is not entirely clear, it has been suggested that CD22 regulates signal transduction of the B cell antigen receptor, B cell migration, and maintenance of peripheral B cell homeostasis and survival. By cross-linking CD22 on tumor cells with CD28 on the T cells, critical CD28 costimulatory signals can be transduced for the enhancement of CD20×CD3 directed T cell activation. In this study it is demonstrated that potent amplification of Odronextamab anti-tumor activity can be achieved by combining with REGN5837 against DLBCL tumors cells, both in vitro and in vivo. Characterization of intratumoral T cell responses reveal that engagement of CD28 in combination with Odronextamab not only mediates curative tumor responses in a preclinical model of DLBCL, but also expands intratumoral T cells and the induction of reprogrammable T cells. Therefore, these data provide support for the potential clinical investigation of REGN5837 in combination with Odronextamab in r/r DLBCL patients.

### Results

### CD28 Expression is Detected on Intratumoral CD8⁺ T Cells at Baseline and Post Odronextamab Treatment in r/r NHL Patients.

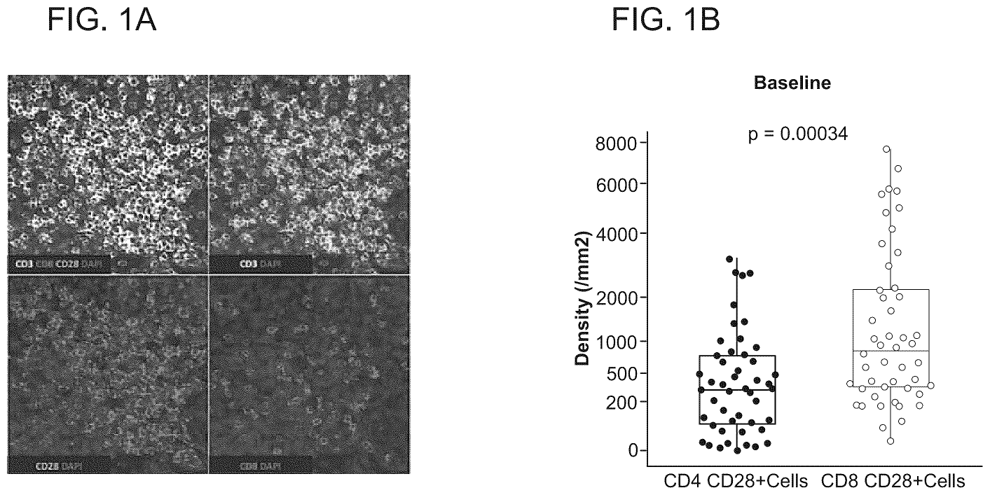

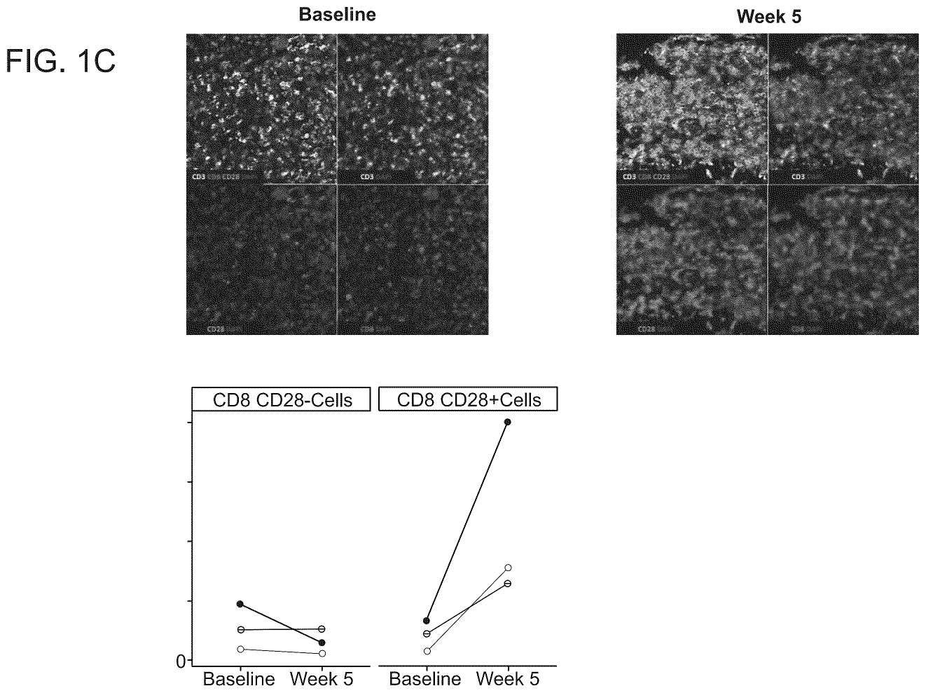

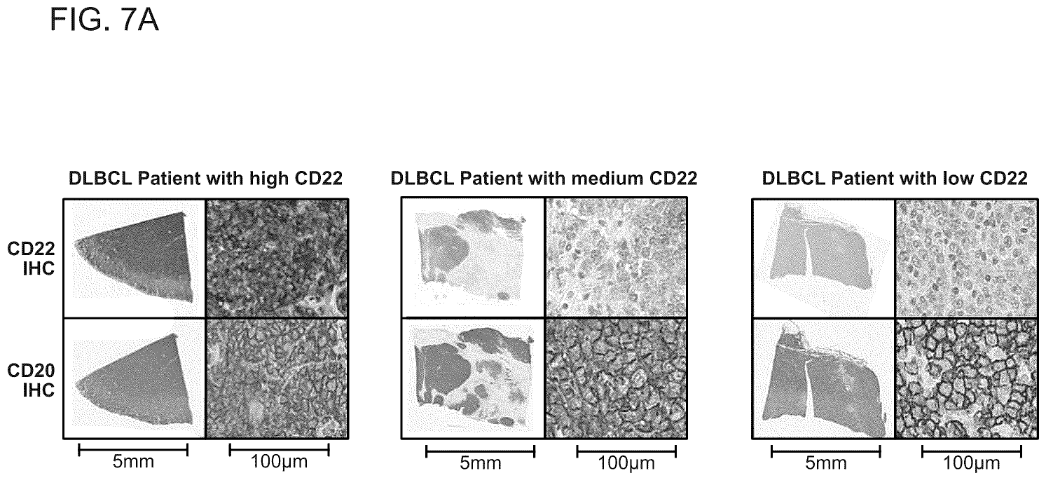

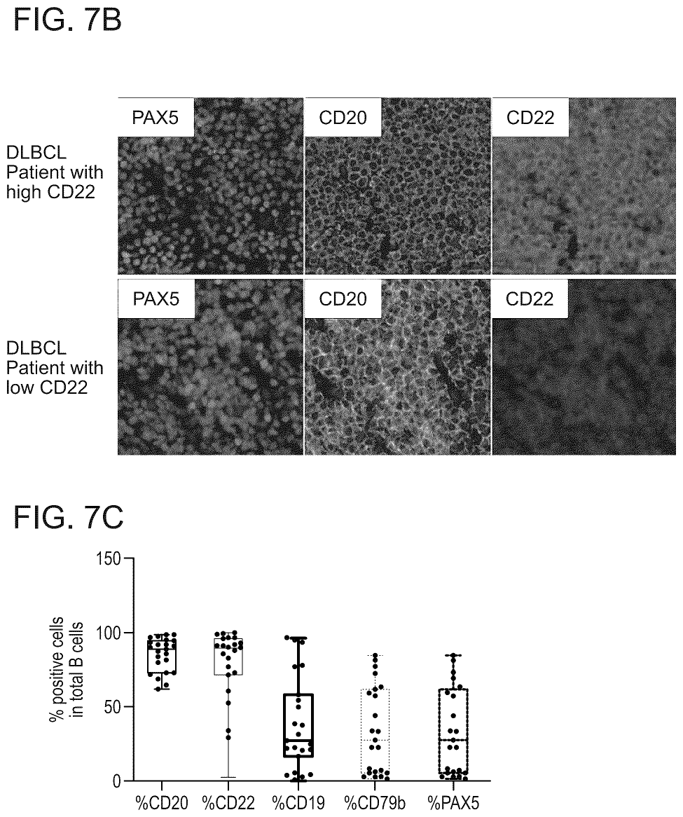

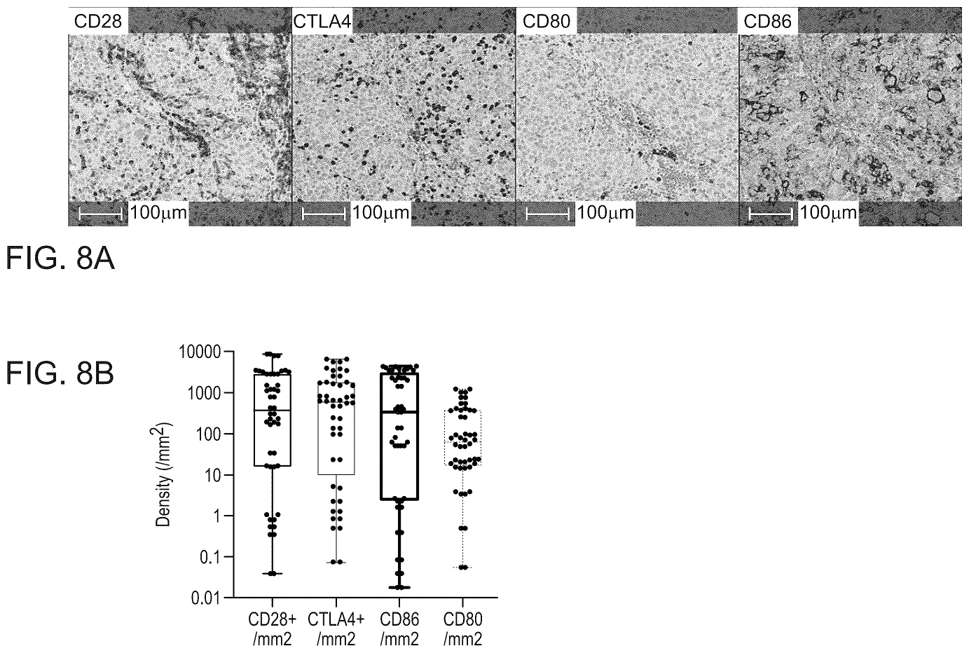

CD22 is a well-validated tumor target for the treatment of B cell leukemia and lymphoma, with broad but variable expression within DLBCL patient samples (FIG. 7A-FIG. 7C). Additionally, it was observed that a significant population of CD28⁺ cells from treatment-naïve resected DLBCL patient samples that can be targeted with the CD22×CD28 bispecific antibody (FIG. 8A and FIG. 8B). Analysis of baseline samples from a dose-finding phase I study (ClinicalTrials.gov Identifier: NCT02290951) for Odronextamab demonstrated that this CD28 expression was preferentially on intratumoral CD8⁺ T cells and to a lesser extent on CD4+ T cells (FIGS. 1A and B). Additionally, while limited in number, several paired patient samples for the presence of CD28⁺CD8⁺ T cells pre- and post-Odronextamab treatment were assessed. A specific increase in the density of CD8⁺CD28⁺ T cells in follicular lymphoma and DLBCL patient samples was observed (FIG. 1C) five weeks post the start of Odronextamab therapy, consistent with the persistence of an intratumoral T cell population that could be targeted by REGN5837 to enhance effector memory differentiation and further augment anti-tumor activity. While CD86 and to a lesser extent CD80 (the two ligands for CD28) were also expressed in resected DLBCL patient samples, concomitant expression of the co-inhibitory molecule CTLA4 was observed (FIG. 8A and FIG. 8B), which has a higher affinity for CD80 and CD86 and can abrogate CD28 costimulatory activity. Therefore, utilizing a CD22×CD28 bispecific antibody may allow for the specific engagement of CD28 and the potential enhancement of Odronextamab activity.

### REGN5837 Augments Odronextamab Mediated T Cell Activation and Effector Function In Vitro.

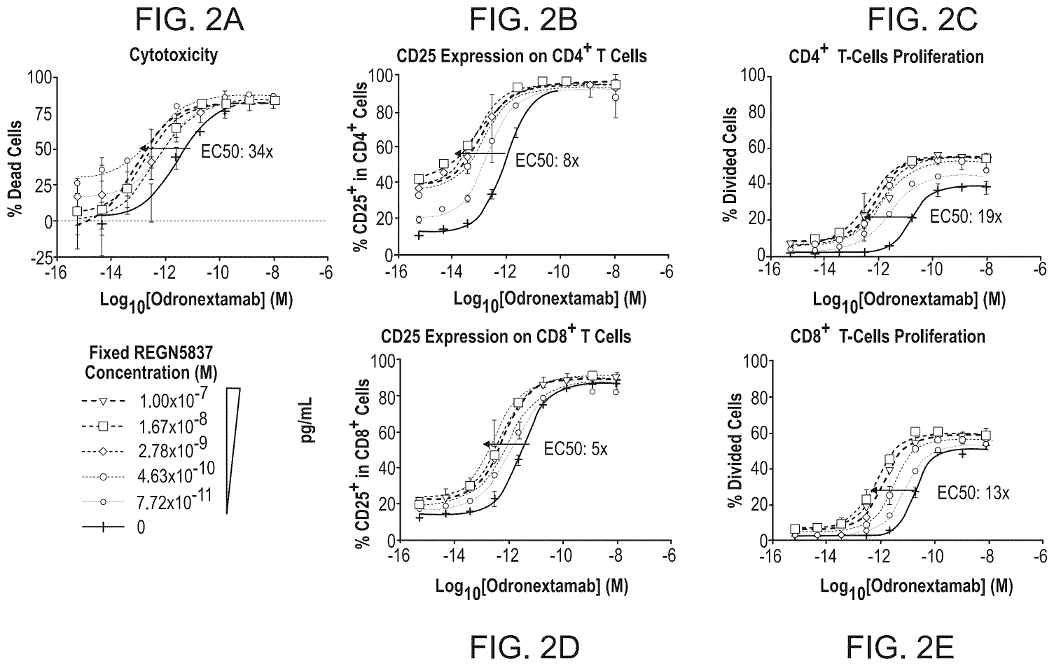

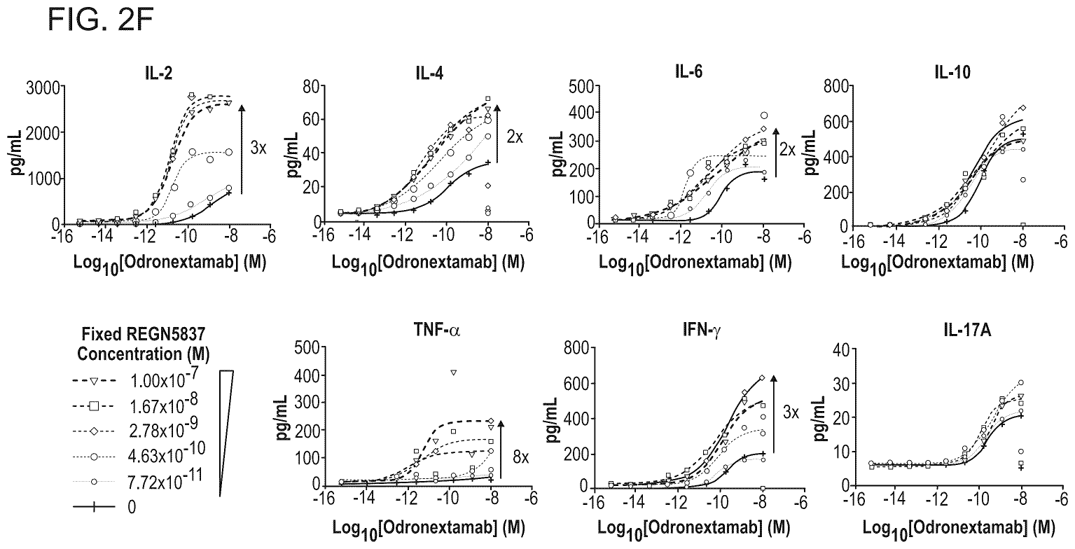

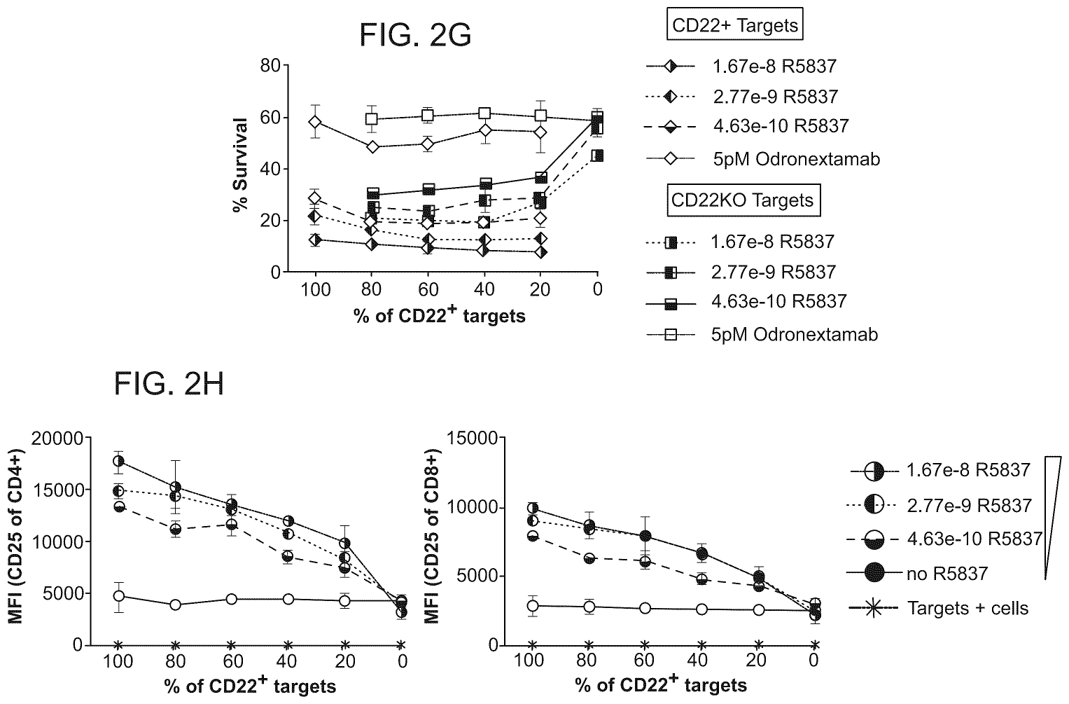

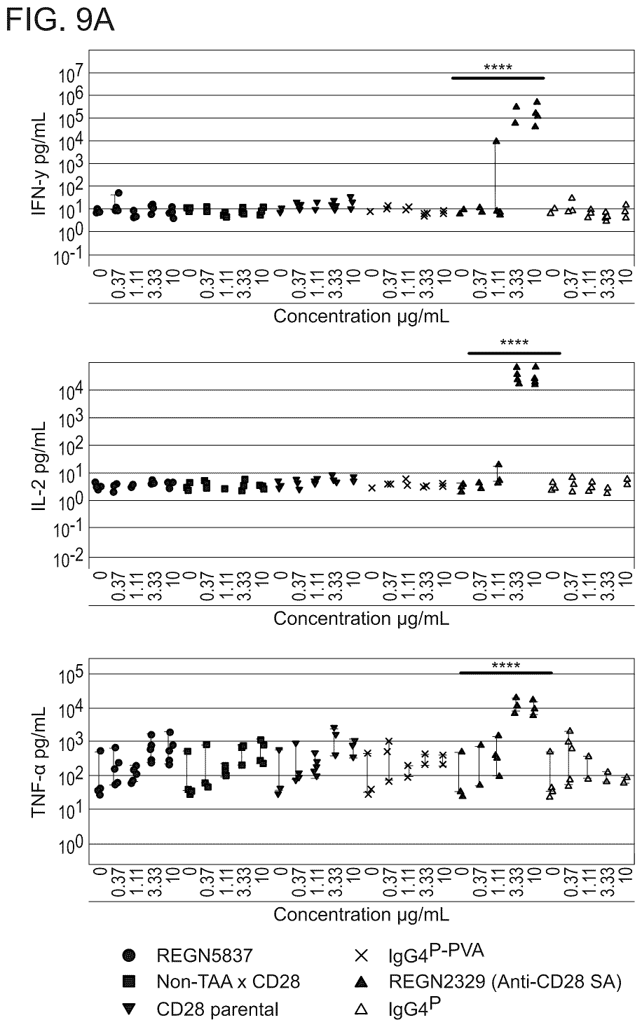

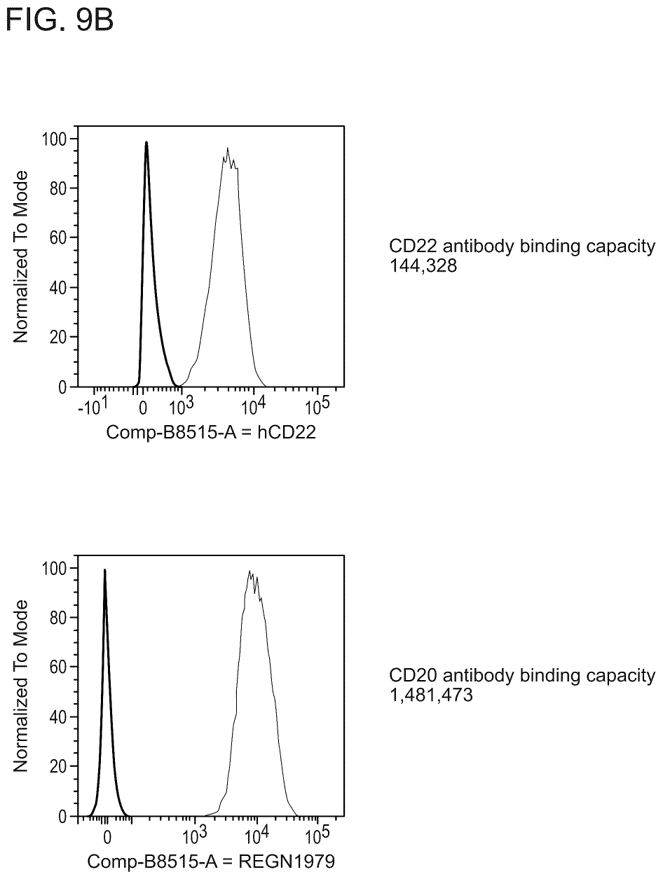

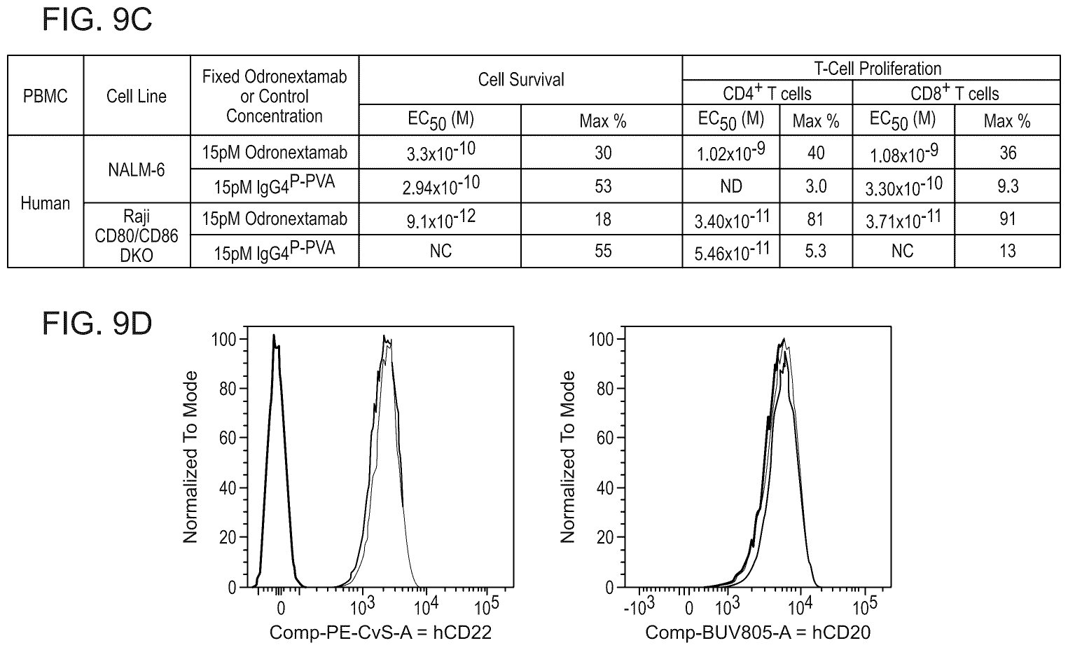

To evaluate the ability of REGN5837 to potentiate the cytolytic and T cell activating activity of Odronextamab, lymphocyte-enriched human peripheral blood mononuclear cells (PBMCs) were co-cultured with WSU-DLCL2 target cells, or DLBCL cells expressing high levels of CD20 and CD22 (FIG. 9B), with a dose titration of Odronextamab (4.8 fM to 10 nM) as a single agent or in the presence of fixed concentrations of REGN5837 (ranging from 77 pM to 10 uM). REGN5837 activity was assayed in the presence of Odronextamab, as the CD28 bispecific cannot mediate costimulatory activity in the absence of signal 1, or engagement of the TCR/CD3 complex, unlike a TeGenero-like CD28 superagonist (FIG. 9A). Administration of REGN5837 costimulation enhanced Odronextamab mediated cytotoxicity of WSU-DLCL2 cells (FIG. 2A), upregulated activation marker CD25 on CD4⁺ (FIG. 2B) and CD8⁺ (FIG. 2D) T cells, and induced proliferation of CD4⁺ (FIG. 2C) and CD8⁺ (FIG. 2E) T cells in a concentration dependent manner (Table 6).

TABLE 6
REGN5837 bispecific antibody enhances Odronextamab
mediated T cell activation and cytotoxicity

| [R5837] | | 1.00E−07 | 1.67E−08 | 2.78E−09 | 4.63E−10 | 7.72E−11 | No R5837 |
|---|---|---|---|---|---|---|---|
| % Cytotocixity | EC50 | 8.53E−14 | 1.85E−13 | 3.98E−13 | 3.46E−13 | 8.37E−13 | 2.89E−12 |
| | Max | 81.49 | 79.75 | 80.55 | 87.49 | 82.07 | 83.19 |
| | Fold (EC50) | 33.9 | 15.7 | 7.3 | 8.3 | 3.5 | 1.0 |
| CD4 T cell | EC50 | 1.16E−13 | 8.70E−14 | 7.11E−14 | 1.46E−13 | 2.08E−13 | 9.17E−13 |
| activation | Max | 94.14 | 95.64 | 94.22 | 91.92 | 91.68 | 92.35 |
| (% CD25) | Fold (EC50) | 7.9 | 10.5 | 12.9 | 6.3 | 4.4 | 1.0 |
| CD8 T cell | EC50 | 6.32E−13 | 5.88E−13 | 2.68E−13 | 9.01E−13 | 1.64E−12 | 3.38E−12 |
| activation | Max | 89 | 90.87 | 89.66 | 86.2 | 88.38 | 87.12 |
| (% CD25) | Fold (EC50) | 5.3 | 5.7 | 12.6 | 3.8 | 2.1 | 1.0 |
| CD4 T cell | EC50 | 8.42E−13 | 9.31E−13 | 4.25E−13 | 1.25E−12 | 2.36E−12 | 1.63E−11 |
| proliferation | Max | 54.72 | 54.09 | 54.79 | 52.06 | 44.73 | 38.67 |
| (% Divided) | Fold (EC50) | 19.3 | 17.5 | 38.3 | 13.1 | 6.9 | 1.0 |
| CD8 T cell | EC50 | 1.55E−12 | 8.31E−13 | 7.09E−13 | 3.43E−12 | 8.36E−12 | 1.97E−11 |
| proliferation | Max | 59.12 | 58.92 | 60.21 | 56.4 | 53.15 | 51.09 |
| (% Divided) | Fold (EC50) | 12.7 | 23.7 | 27.8 | 5.7 | 2.4 | 1.0 |

Analysis of supernatant from the co-cultures revealed that REGN5837 also augmented Odronextamab mediated cytokine release (FIG. 2F) in a dose dependent manner (Table 7). The capacity of REGN5837 to enhance Odronextamab activity was additionally demonstrated with B cell acute lymphoblastic leukemia (NALM6) and Burkitt's lymphoma (Raji CD80/Cd86dko) cell lines as targets (FIG. 9C).

TABLE 7
REGN5837 bispecific antibody enhances cytokine production by Odronextamab treatment.

| [R5837] | | 1.00E−07 | 1.67E−08 | 2.78E−09 | 4.63E−10 | 7.72E−11 | No R5837 |
|---|---|---|---|---|---|---|---|
| IL-2 | EC50 | 1.66E−11 | 1.53E−11 | 1.28E−11 | 1.89E−11 | 3.77E−10 | 9.997E−10 |
| | Max (pg/ml) | 2637 | 2793 | 2705 | 1560 | 935 | 775 |
| | Fold (max) | 3.4 | 3.6 | 3.5 | 2.0 | 1.2 | 1.0 |
| IL-4 | EC50 | 2.81E−11 | 1.95E−11 | 5.18E−12 | 5.36E−11 | 3.89E−08 | 1.25E−10 |
| | Max (pg/ml) | 79.32 | 75.35 | 63.24 | 70.29 | 116.3 | 36.17 |
| | Fold (max) | 2.2 | 2.1 | 1.7 | 1.9 | 3.2 | 1.0 |
| IL-6 | EC50 | 5.13E−11 | 7.89E−12 | 2.68E−11 | 1.45E−12 | 1.61E−11 | 7.42E−11 |
| | Max (pg/ml) | 366.2 | 303.8 | 367.1 | 245 | 210.3 | 189.8 |
| | Fold (max) | 1.9 | 1.6 | 1.9 | 1.3 | 1.1 | 1.0 |
| IL-10 | EC50 | 3.54E−11 | 1.04E−10 | 2.43E−10 | 5.29E−11 | 3.34E−11 | 9.95E−11 |
| | Max (pg/ml) | 527.7 | 682.5 | 835 | 633.3 | 449.9 | 511.8 |
| | Fold (max) | 3.0 | 1.3 | 1.6 | 1.2 | 0.9 | 1.0 |
| TNFa | EC50 | 5.03E−12 | 8.69E−13 | 3.45E−12 | 2.08E−03 | 1.62E−10 | 2.43E−10 |
| | Max (pg/ml) | 235.5 | 129.2 | 170.3 | NC | 35.35 | 29.6 |
| | Fold (max) | 8.8 | 4.4 | 5.8 | NC | 1.2 | 1.0 |
| IFNg | EC50 | 1.17E−10 | 5.34E−11 | 2.00E−10 | 6.72E−11 | 6.57E−11 | 1.82E−10 |
| | Max (pg/ml) | 525.3 | 536.8 | 693.1 | 348.4 | 156.5 | 205.5 |
| | Fold (max) | 2.6 | 2.6 | 3.4 | 1.7 | 0.8 | 1.0 |
| IL-17a | EC50 | 3.22E−10 | 1.11E−10 | 7.60E−11 | 4.63E−10 | 1.86E−10 | 2.50E−30 |
| | Max (pg/ml) | 26.59 | 25.23 | 20.56 | 33.31 | 21.92 | 20.52 |
| | Fold (max) | 1.3 | 1.2 | 1.0 | 1.6 | 1.1 | 1.0 |

As CD22 expression is variable within DLBCL patient samples with tumors containing a mixture of CD22⁺ and CD22⁻ tumor cells (FIG. 1A-C), the ability of REGN5837 to augment the ability of T cells to kill CD22⁺ and CD22⁻ targets was also evaluated. Purified T cells were incubated with CRISPR-edited CD22 deficient and CD22 wild-type WSU-DLCL2 cells and stimulated with a fixed concentration of Odronextamab (5 pM) and REGN5837 ranging from 4.63×10⁻¹⁰ to 1.67×10⁻⁸ M. Culturing T cells with target cells expressing CD22⁺ and REGN5837 augmented Odronextamab-mediated tumor cell lysis; while as expected, culturing T cells with target cells that do not express CD22 did not result in enhanced cytotoxicity (FIG. 2G). However, culturing T cells with a mixed population consisting of 20-80% CD22⁺ targets resulted in increased killing of both the CD22⁺ and CD22⁻ target populations (FIG. 2G) and increased CD4 and CD8 T cell activation (FIG. 2H) in a REGN5837 dose dependent manner. While CD22⁻ target cells were not lysed at the same magnitude as CD22⁺ target cells at the highest concentration of REGN5837 evaluated (11% survival of CD22⁺ cells vs. 21% survival of CD22⁻ cells when mixed at a 4:1 ratio in comparison to 60% survival without REGN5837 treatment), the presence of CD22⁺ targets was able to enhance the killing of the CD22⁻ cells even at the lowest proportion of CD22⁺/CD22^{- cells evaluated. Therefore, these data demonstrate that variability in CD}22 expression on DLBCL tumor cells does not preclude REGN5837 from augmenting the in vitro cytotoxic activity of Odronextamab.

### REGN5837 Mediated Costimulation Enhances Odronextamab Anti-Tumor Efficacy in a Xenogeneic DLBCL Tumor Model.

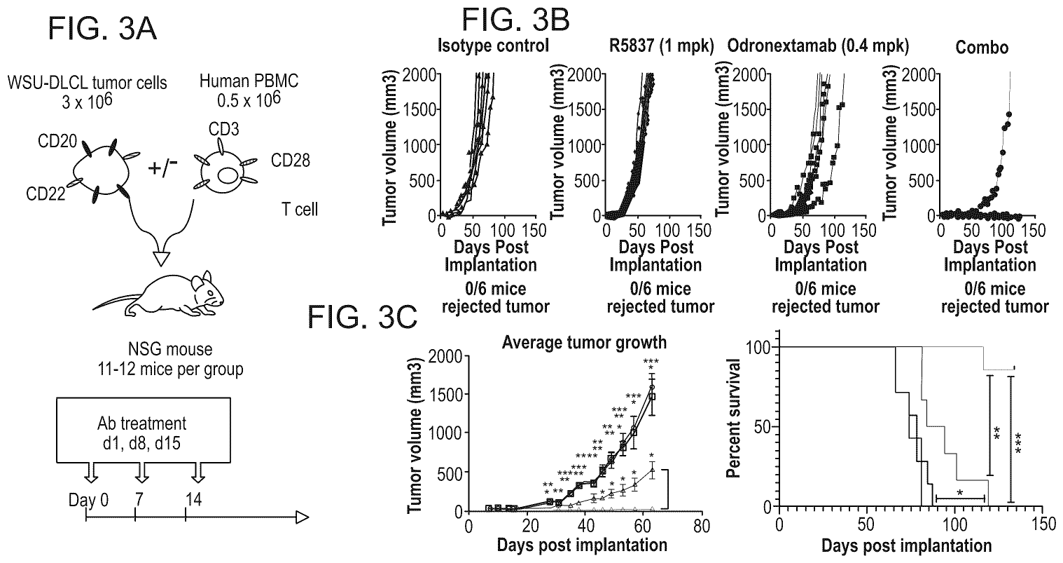

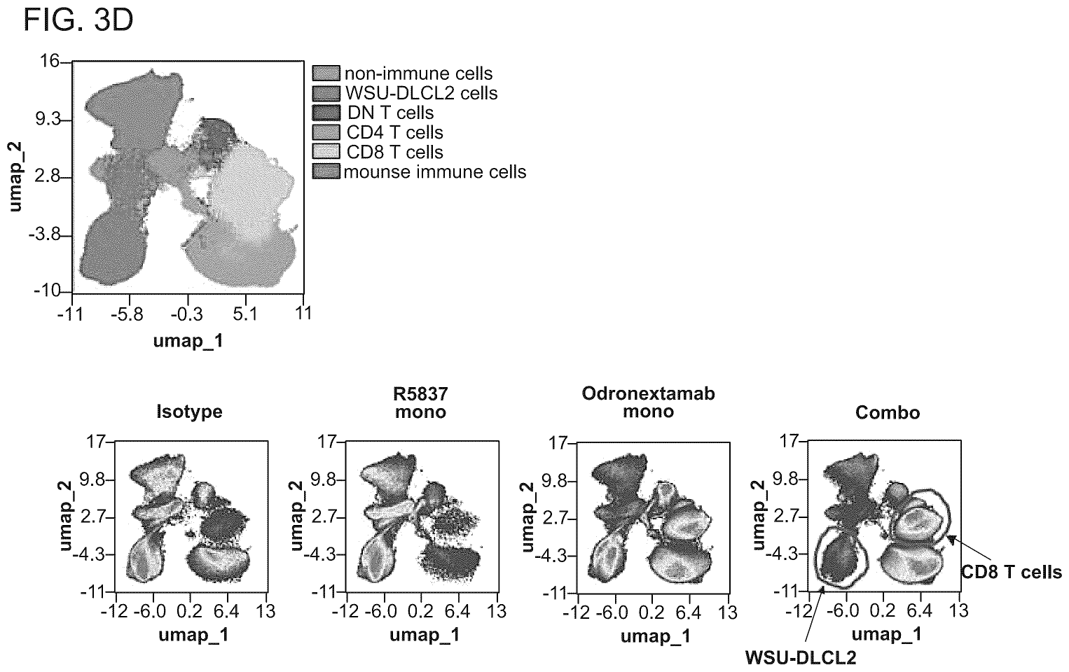

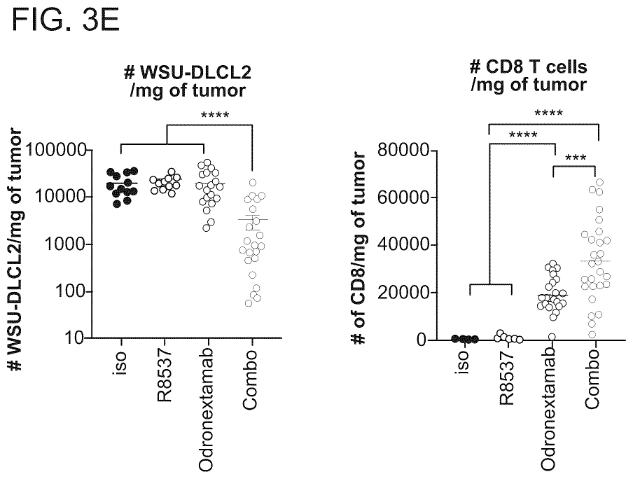

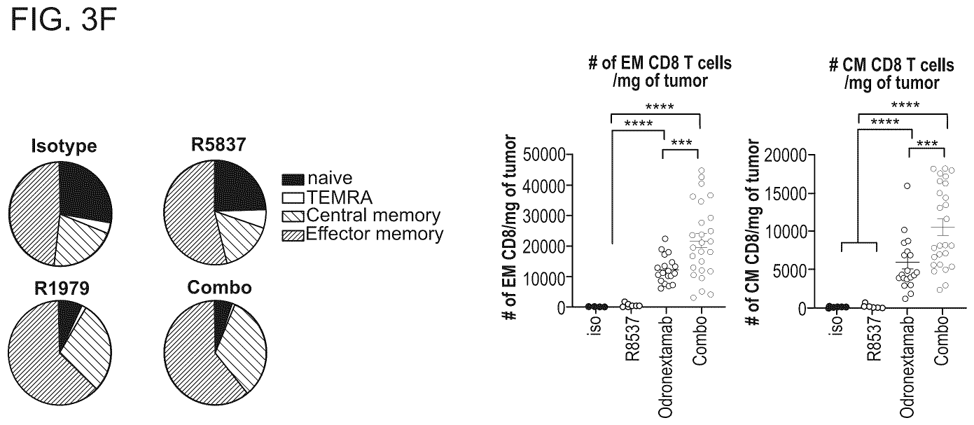

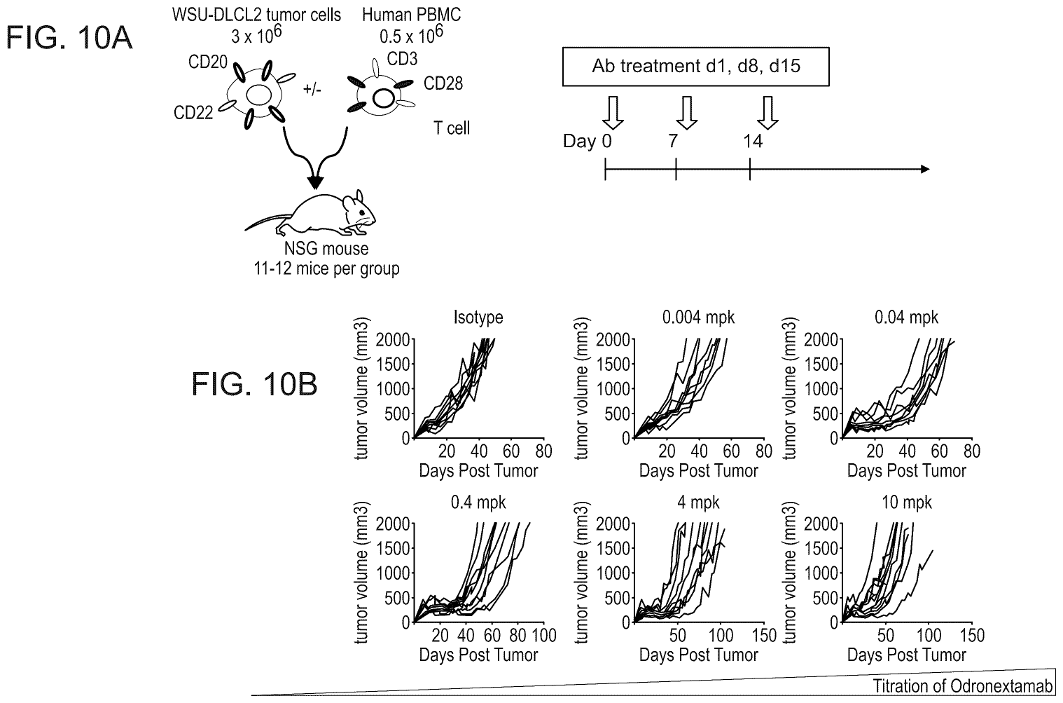

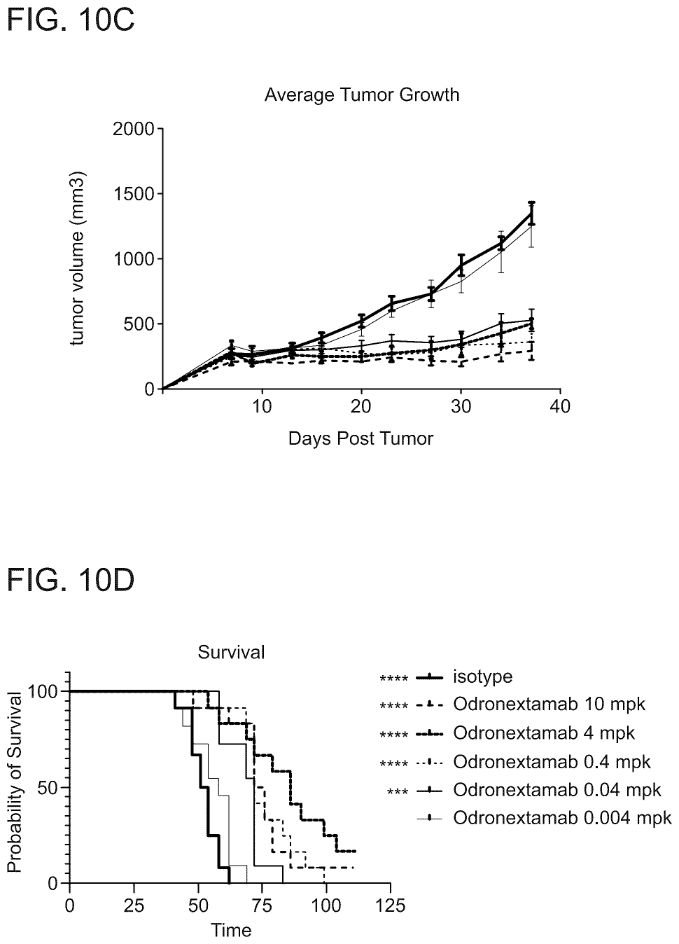

The ability of REGN5837 to augment the anti-tumor activity of Odronextamab in vivo was evaluated in the WSU-DLCL2 tumor model, a xenogeneic DLBCL tumor where Odronextamab alone was not sufficient to mediate tumor clearance, thereby modeling patients who lack a complete response to CD3 bispecific antibody treatment. Dose titration of Odronextamab revealed that while the WSU-DLCL2 tumor model was initially responsive to CD20×CD3 mediated killing in vivo (FIG. 10B and FIG. 10C), this treatment was not able to mediate sustained tumor rejection, with all animals eventually succumbing to tumor burden despite treatment with high-dose Odronextamab (10 mg/kg) (FIGS. 10B and 10D). Therefore, it was evaluated whether the addition of REGN5837 could further promote anti-tumor activity (FIG. 3A). As expected, prophylactic treatment with REGN5837 alone did not have any effect on tumor growth (FIG. 3B, C). While Odronextamab monotherapy initially suppressed tumor growth (FIG. 3B, C left) and conferred survival advantage in comparison to isotype control treated animals (FIG. 3C right), all animals eventually succumbed to tumor outgrowth. However, the combination of the REGN5837 with Odronextamab augmented anti-tumor immunity resulting in 86% (6/7) of the animals rejecting their tumors (FIG. 3B) and significantly extending overall survival of animals over the monotherapy treated groups (FIG. 3C right).

### CD22×CD28 Mediated Costimulation Enhances CD20×CD3 Anti-Tumor Efficacy Against Preclinical Models of B Cell Malignancies

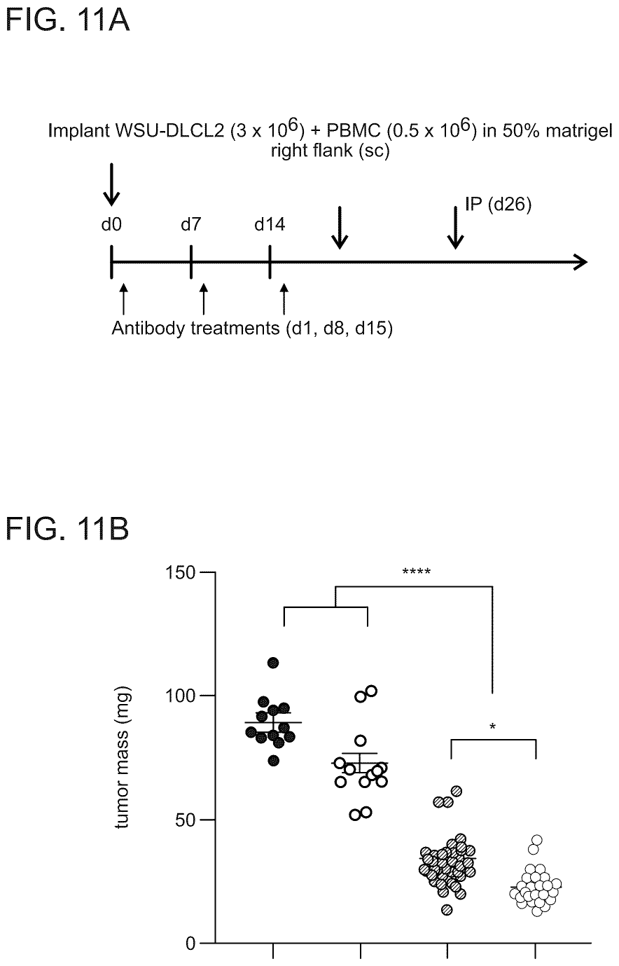

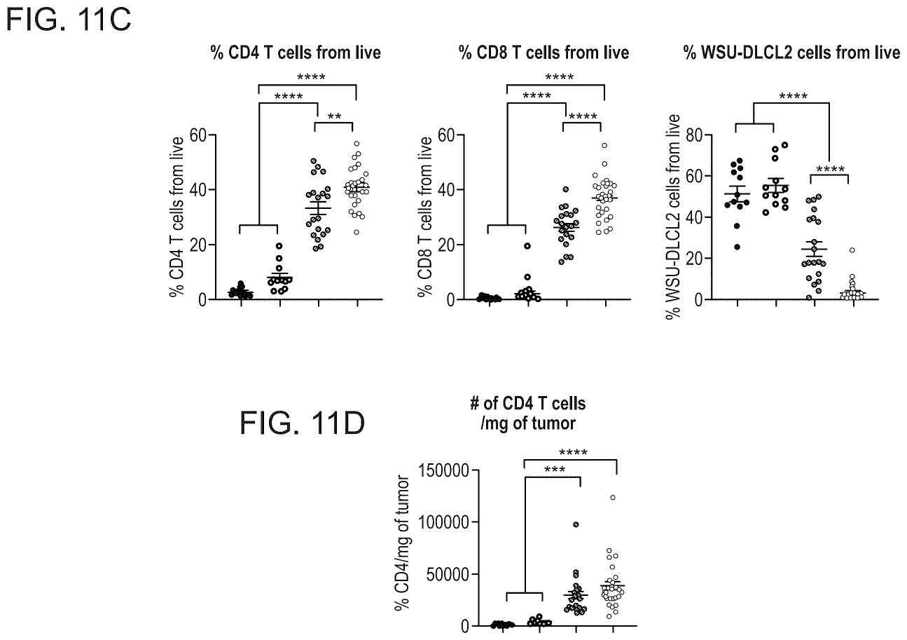

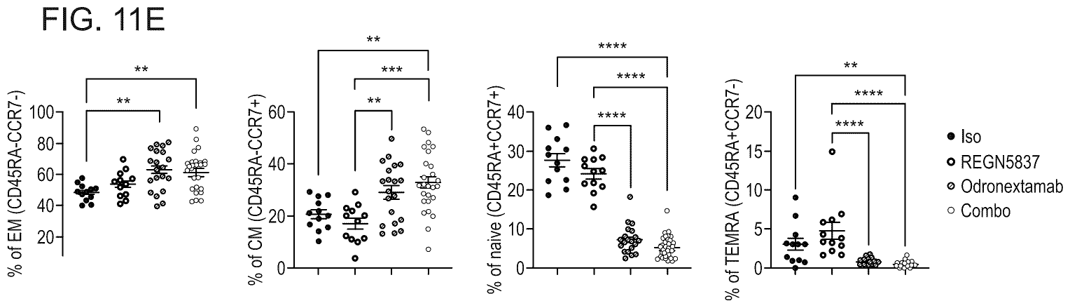

To investigate how combination treatment augments anti-tumor immunity, different treatments were evaluated in the modulatation of intratumoral T cell responses 26 days post implantation (FIG. 11A). At this time point, Odronextamab treatment significantly decreased tumor mass and combination treatment further suppressed tumor growth (FIG. 11B). High dimensional reduction analysis of intratumoral immune subsets (FIG. 3D) revealed the enrichment and depletion of certain populations in response to CD3 bispecific or combination treatment. Administration of Odronextamab treatment preferentially expanded intratumoral CD4⁺ and CD8⁺ T cells while decreasing the percentage of WSU-DLCL2 tumor cells (FIG. 11C). The addition of REGN5837 to Odronextamab treatment further increased the frequency of T cells with a concurrent decrease in the frequency of tumor cells in comparison to monotherapy treatment with the CD3 bispecific (FIG. 11C). The density of tumor and T cell subsets was enumerated and it was revealed that Odronextamab monotherapy significantly increased the intratumoral density of CD4⁺ (FIG. 11D) and CD8⁺ T cells (FIG. 3E). Combination therapy further increased the density of CD8⁺ T cells and concomitantly decreased the number of WSU-DLCL2 cells/mg of tumor in comparison to Odronextamab alone (FIG. 3E). Examining the induction of memory subsets revealed that treatment with Odronextamab significantly drove the expansion of effector and central memory CD8 T cells (FIG. 3F left) and CD4 T cells (FIG. 11E) in comparison to isotype control. Interestingly, the addition of REGN5837 significantly increased the density of central and effector memory CD8⁺ T cells in comparison to Odronextamab or CD22×CD28 monotherapy treated groups (FIG. 3F right), driven by the increased density of the CD8⁺ T cell population (FIG. 3E right). Combination treatment did not further change the proportion of memory to naïve cells.

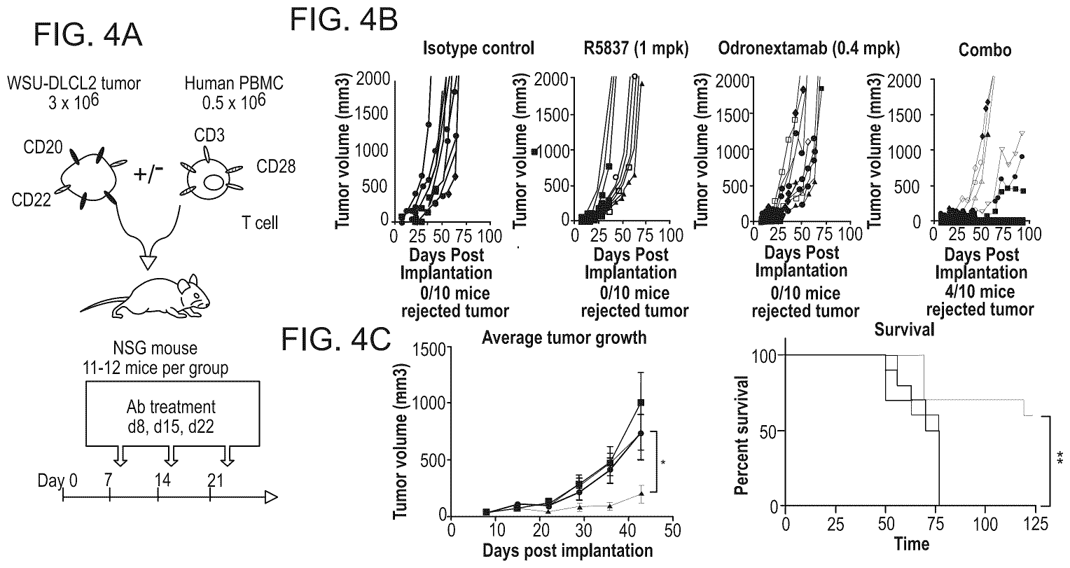

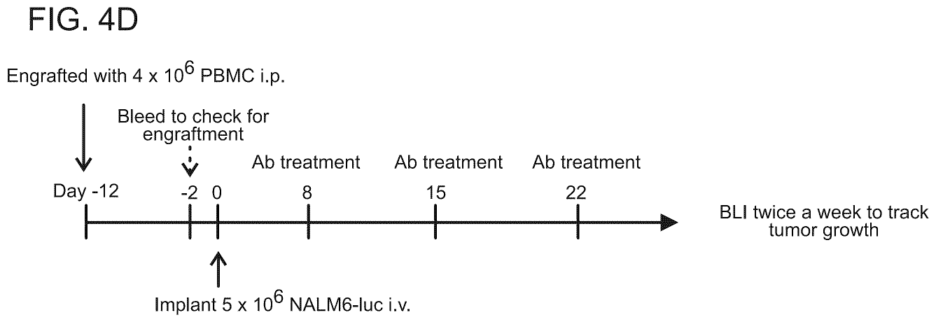

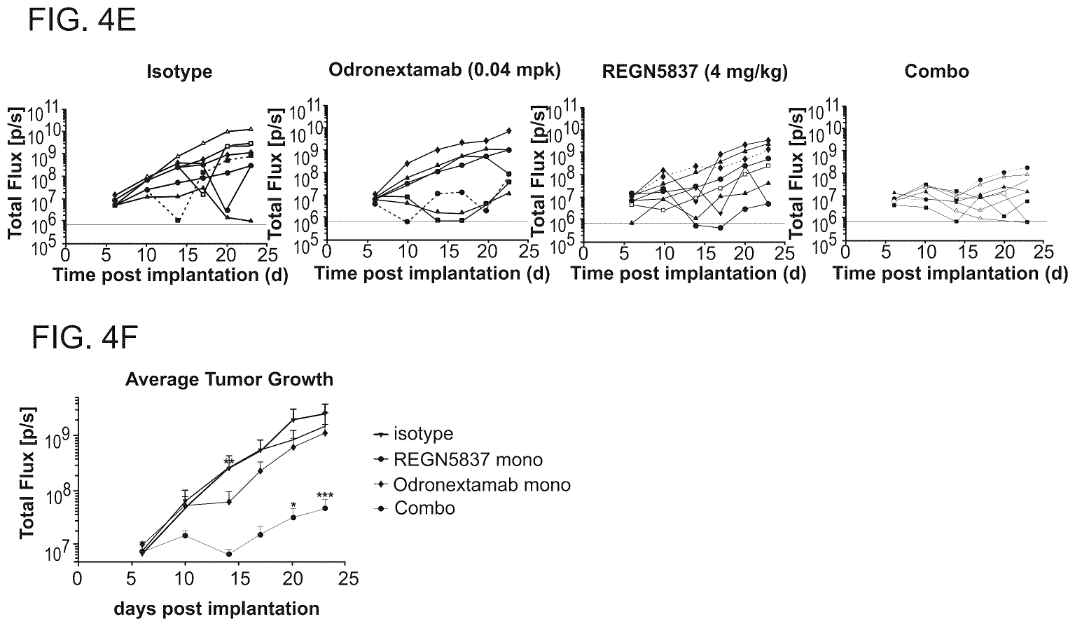

As potent synergy was observed with the combination of REGN5837 with Odronextamab for the prophylactic treatment of WSU-DLCL2 tumors, it was decided to examine combinatorial efficacy in the context of delayed or therapeutic treatment. Delaying investigational treatment by a week abrogated the monotherapy activity of Odronextamab (FIG. 4B, C) as there was no longer suppression of tumor growth. However, combinatorial treatment still mediated potent anti-tumor activity with significant inhibition of tumor growth (FIG. 4C) and an overall rejection rate of 40% which was associated with a significant survival benefit (50% survival at 125 days post tumor implantation) in comparison to Odronextamab alone (0% survival at 75 days). Additionally, the efficacy of REGN5837 was evaluated for the enhancement of Odronextamab activity in a systemic tumor model of B cell acute lymphoblastic leukemia (B-ALL). Immunodeficient animals were pre-engrafted with human PBMCs and implanted 12 days later intravenously with NALM-6 B-ALL cells engineered to express luciferase to allow for in vivo tracking using bioluminescence imaging (BLI). Delaying treatment to 8 days post implantation resulted in a non-significant trend towards decreased tumor burden in the Odronextamab treated group (FIG. 4E and FIG. 4F). However, the combination of REGN5837 with Odronextamab induced a significant suppression of tumor growth (FIG. 4F).

Combination of REGN5837 with Odronextamab Enhances Peripheral and Intratumoral CD8⁺ T Cell Responses in Human Immune System Reconstituted Animals Bearing DLBCL Tumors

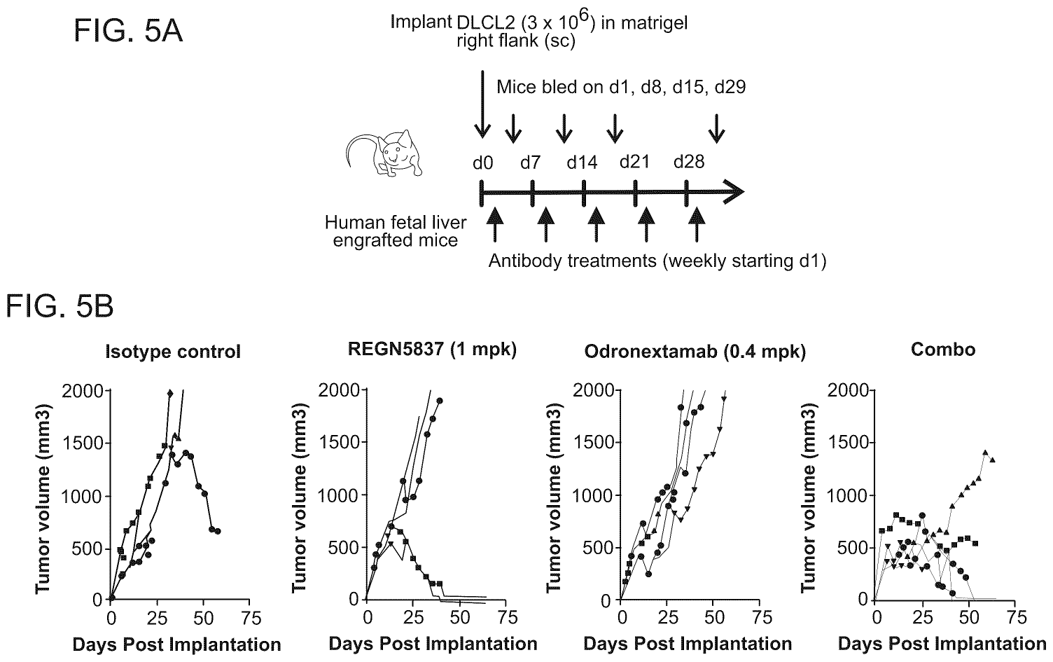

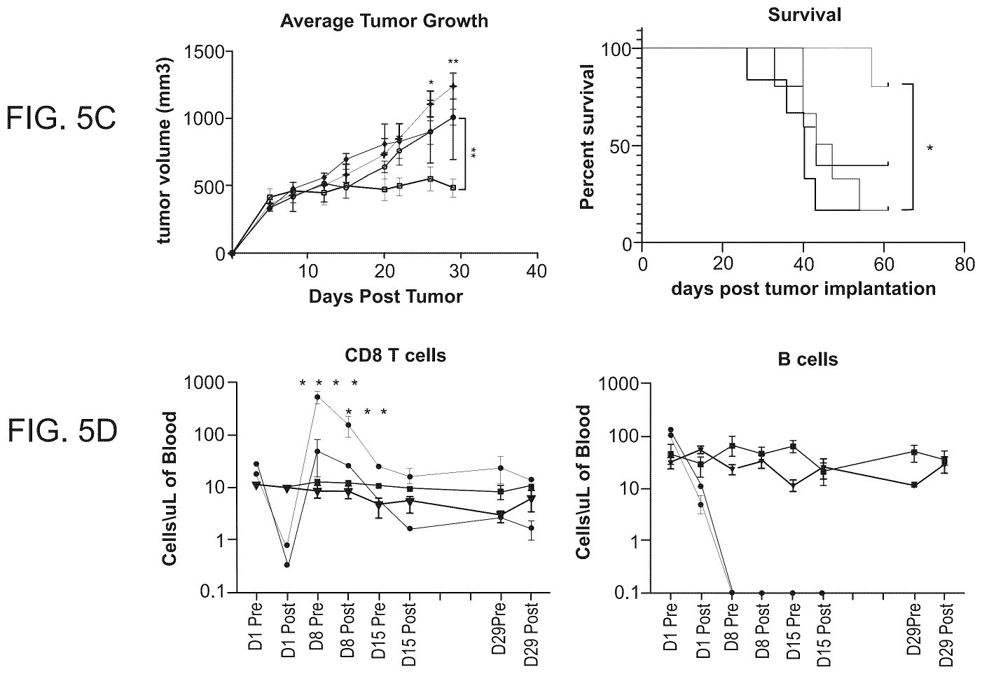

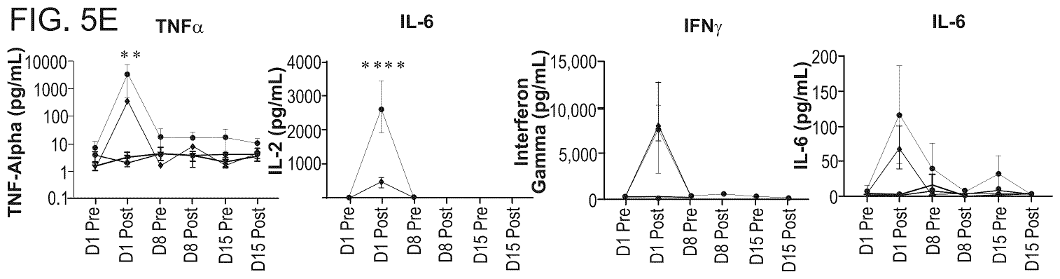

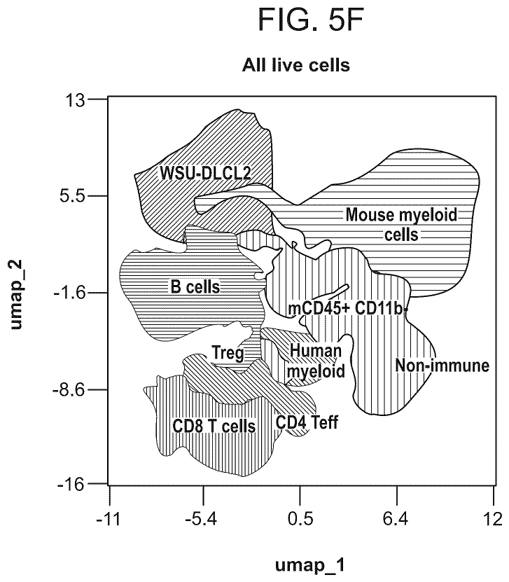

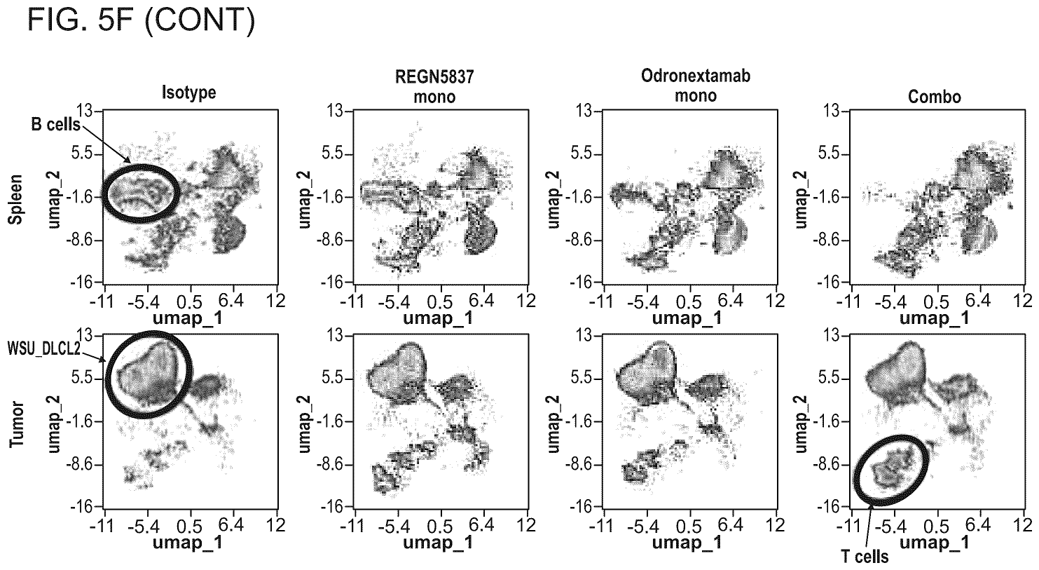

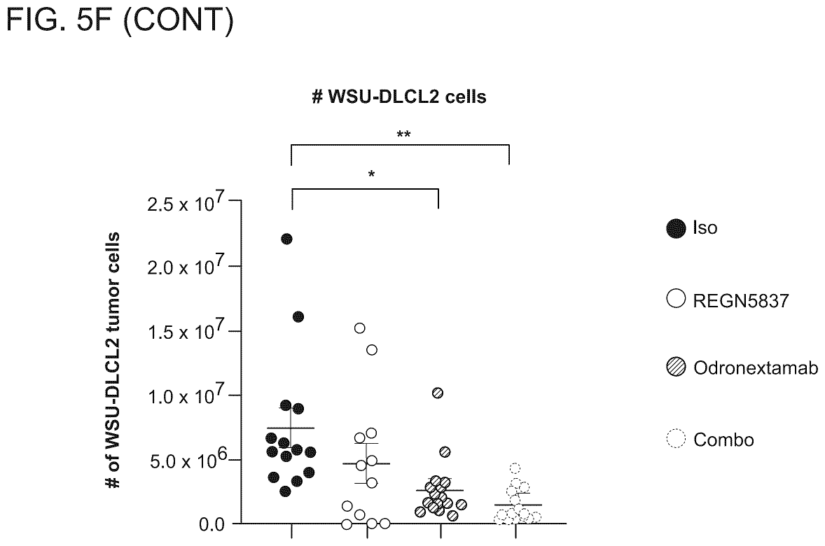

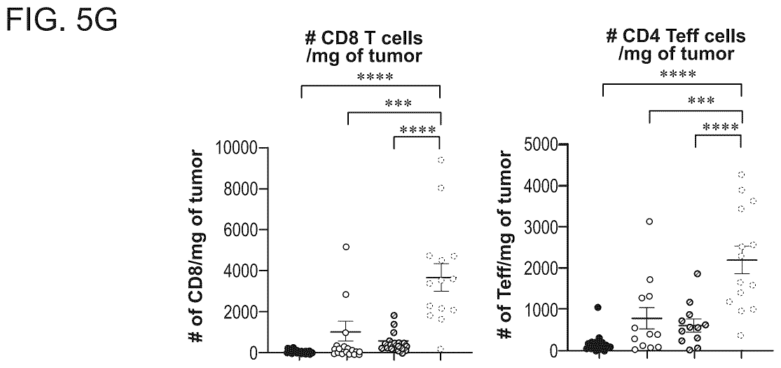

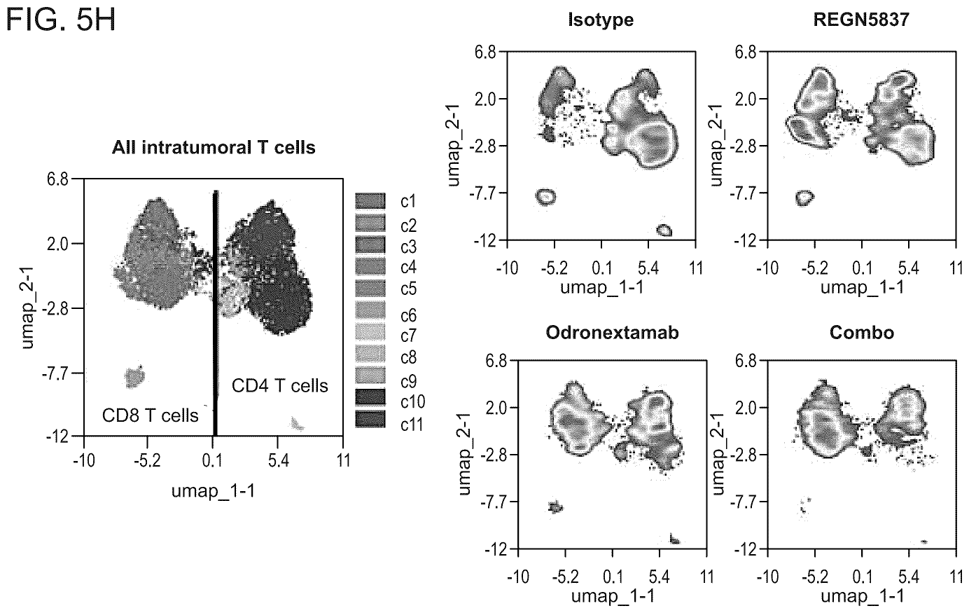

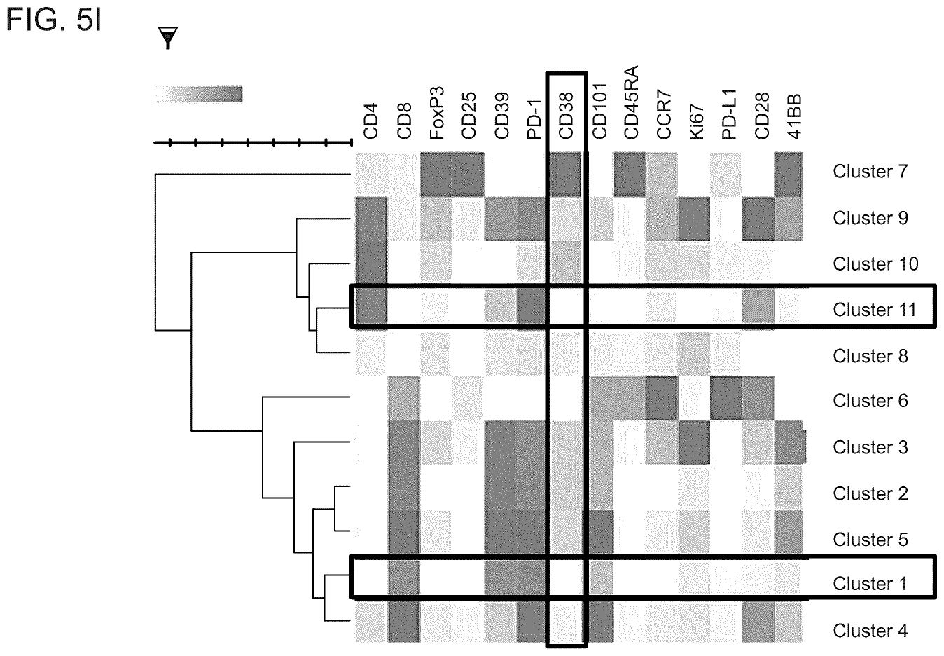

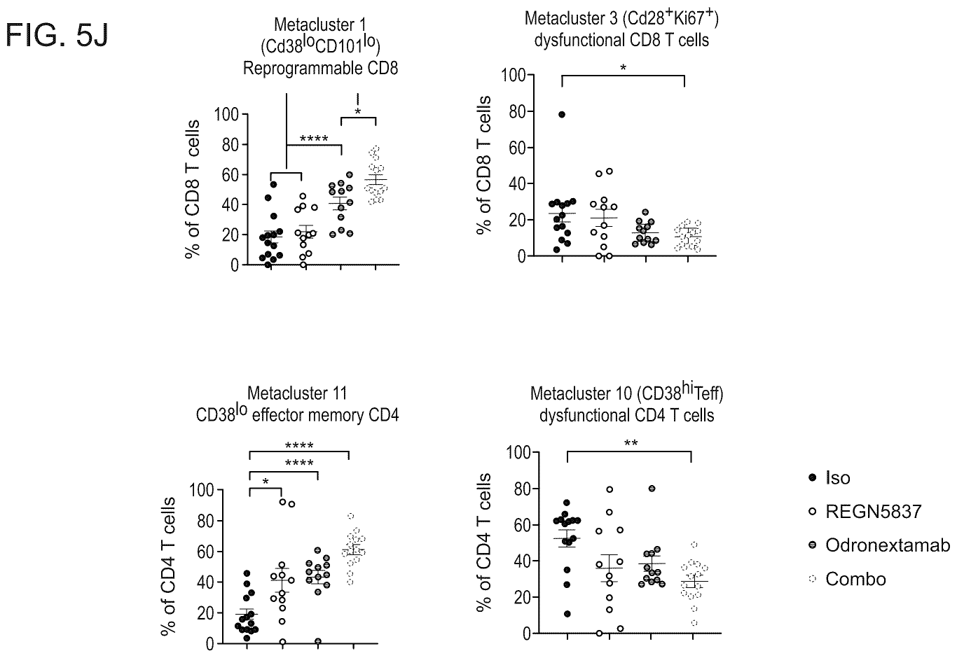

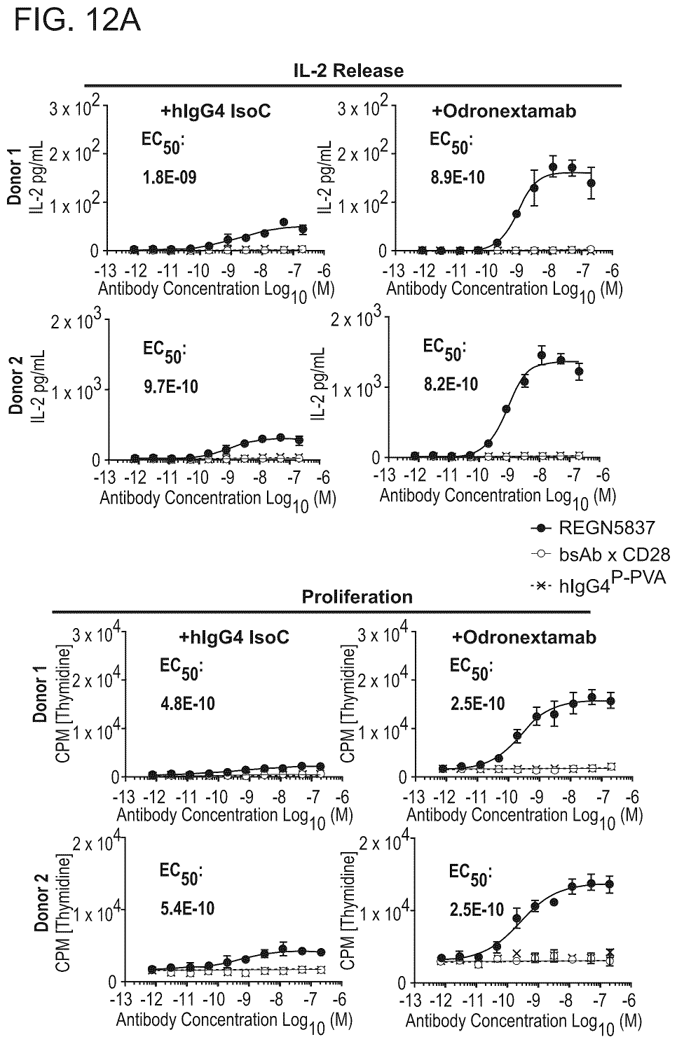

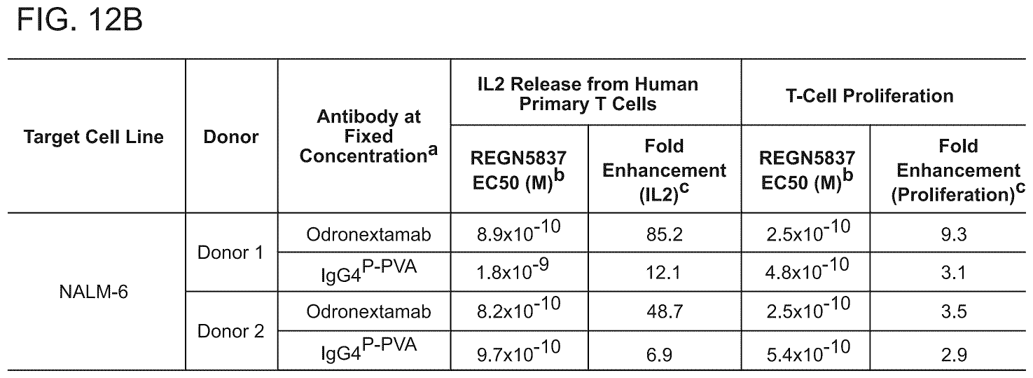

In order to evaluate the in vivo efficacy of REGN5837 in a more physiologically relevant setting, WSU-DLCL2 tumors were subcutaneously implanted into SIRPA^{h/h} TPO^{h/m} Rag2^{-/-} Il2rg^{-/-} mice, or human immune system (HIS) animals, that were engrafted with fetal liver CD34⁺ cells. Animals were randomized into indicated treatment groups based on the fetal liver donor, human T cell engraftment frequency, and sex. Monotherapy with Odronextamab at 0.4 mpk did not suppress tumor growth, while combination treatment with REGN5837 significantly inhibited tumor growth (FIG. 5C left) and enhanced survival (80% survival at 64 days post implantation) in comparison to the CD3 bispecific antibody alone (0% survival at 64 days post implantation) or isotype control (0% survival at 61 days post implantation) (FIG. 5C right). REGN5837 monotherapy also induced tumor rejection (FIG. 5B and FIG. 5C) as underlying this anti-tumor response was the allogeneicity between donor T cells and WSU-DLCL2 tumor cells which provides “signal 1”. In vitro, REGN5837 enhanced T cell activation and effector function mediated by an allogeneic signal 1 (FIG. 12A and FIG. 12B); therefore, this increased anti-tumor activity in vivo may be attributed to the costimulation of an allogeneic response.

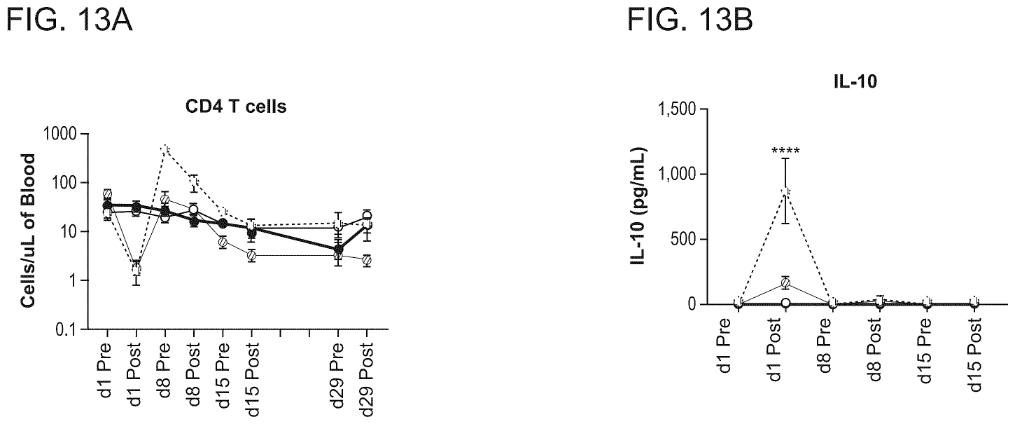

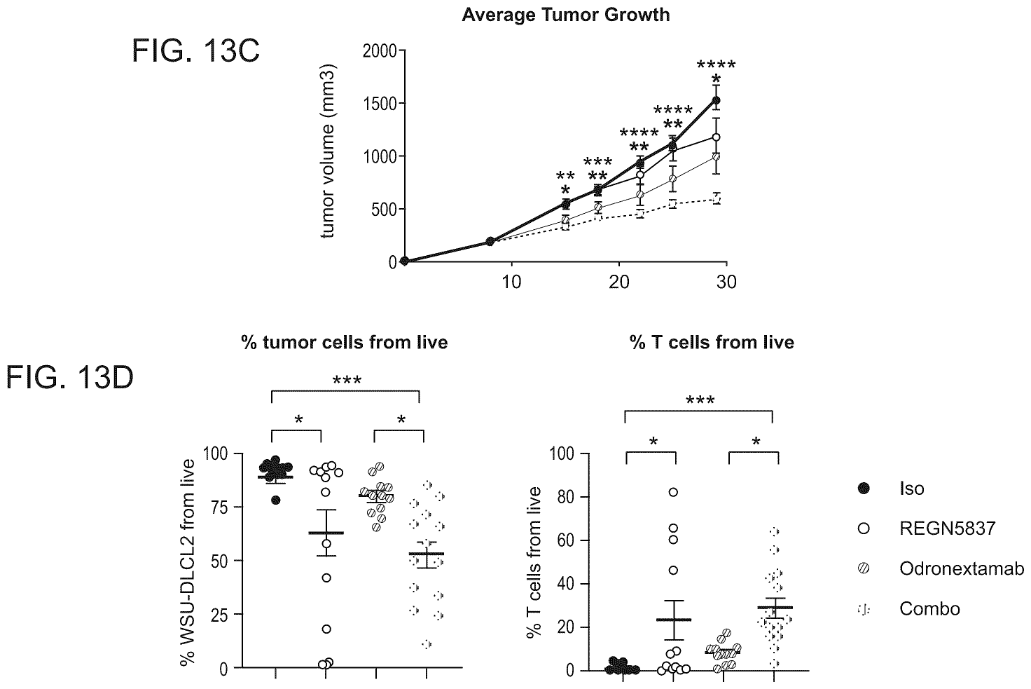

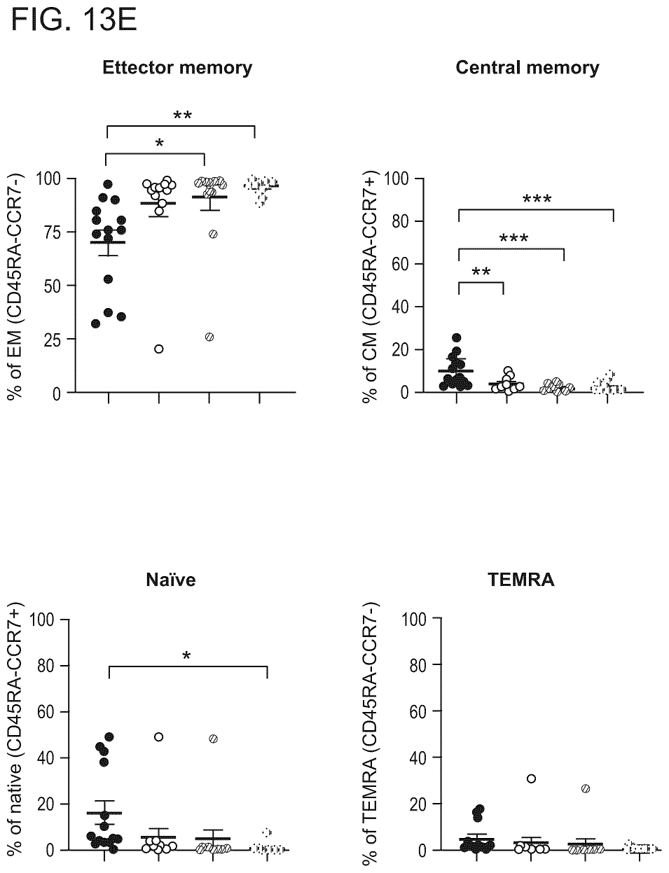

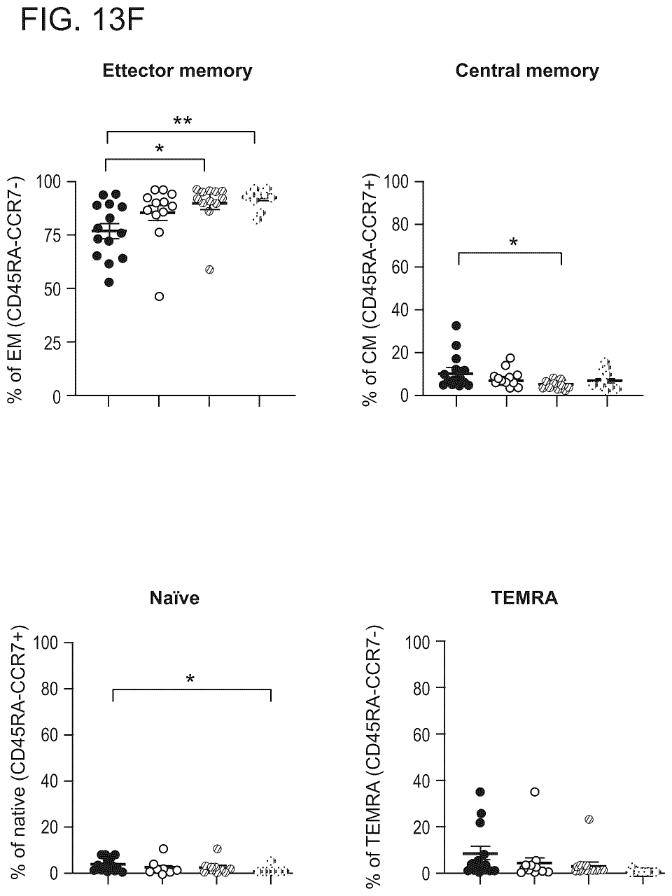

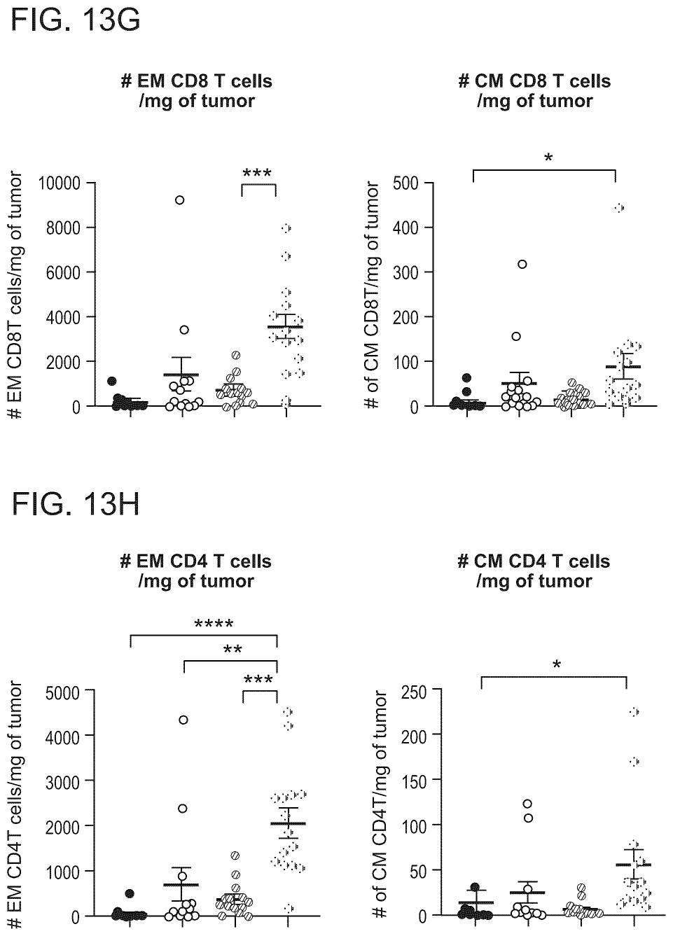

In order to examine peripheral T cell activation and the potential for enhanced cytokine release after combination treatment, serum cytokines were evaluated. Tumor bearing human immune system mice were bled pre- and post-dosing for the evaluation of lymphocyte subsets and the induction of serum cytokines. As expected, monotherapy treatment with REGN5837 did not exhibit single agent activity in the peripheral blood as there was no discernable T cell activation post treatment. However, monotherapy treatment with Odronextamab efficiently depleted circulating B cells (FIG. 5D right) and increased CD8⁺ (FIG. 5D left) T cells in the blood after an initial T cell margination which has also been observed in response to other CD3 bispecific antibodies in preclinical and clinical studies. The combination of REGN5837 with Odronextamab further promoted the expansion of CD8⁺ (FIG. 5D left) and CD4⁺ (FIG. 13A) T cells in the blood in comparison to Odronextamab monotherapy. As expected, examination of serum cytokines post dosing revealed that REGN5837 did not induce any serum cytokines as there was no signal 1 from either an allogeneic tumor or from CD20×CD3 treatment. However, an induction of serum cytokines post first dose by Odronextamab treatment in agreement with previous studies was observed (FIG. 5E and FIG. 13B). Combinatorial treatment induced a significant post first dose cytokine release (TNFa, IL-2, IL-10) in comparison to Odronextamab monotherapy, while there was a trend towards increased IL-6. Overall, the increased, but transient induction of serum cytokines and expansion of T cells in the peripheral blood is evidence of enhanced T cell activation induced by CD28 costimulation.

Combination of REGN5837 with Odronextamab Enhances Anti-Tumor Activity by Augmenting Intratumoral T Cell Accumulation and Skewing T Cells Towards a Reprogrammable State and Away from a Dysfunctional Phenotype.

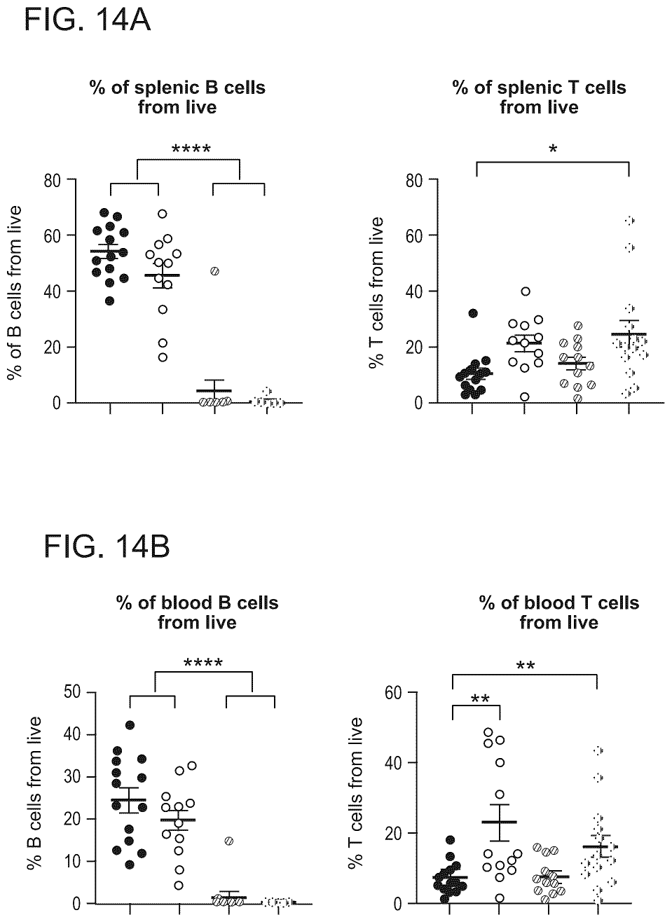

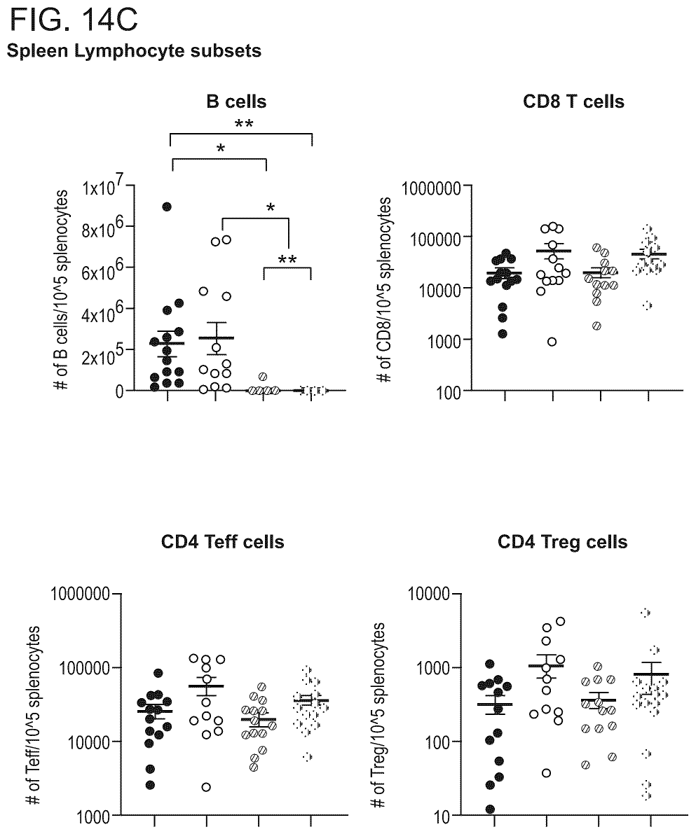

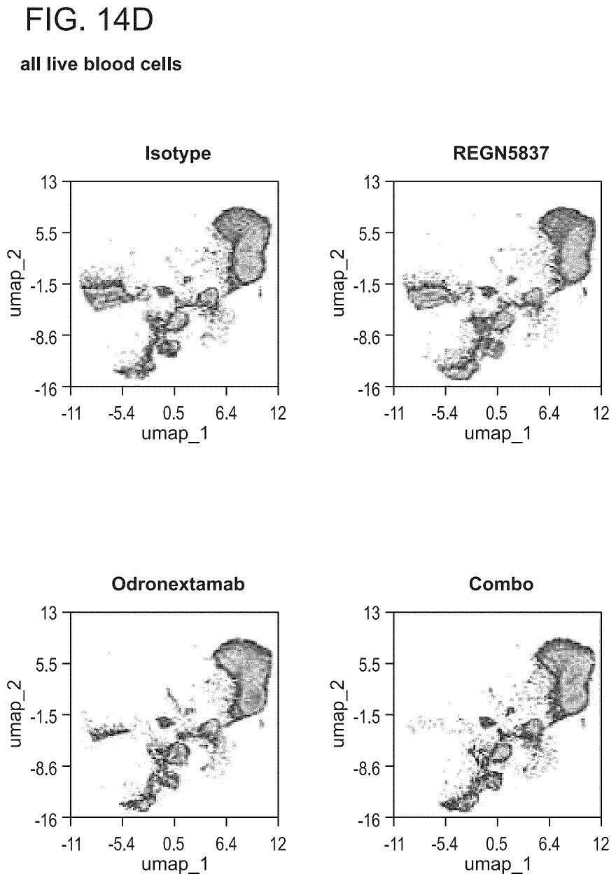

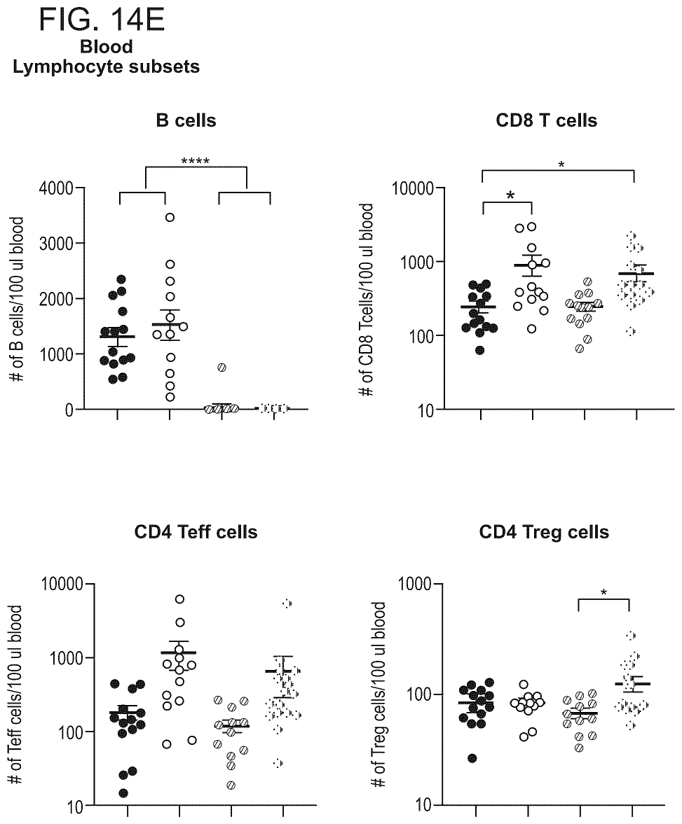

To determine how REGN5837 was enhancing the anti-tumor activity of Odronextamab, intratumoral, splenic, and blood T cell responses were immunoprofiled 30 days post implantation. At this time point, the combination of Odronextamab and REGN5837 significantly suppresses tumor growth in comparison to the isotype and REGN5837 monotherapy treated animals with a trend towards decreased tumor growth in comparison to Odronextamab monotherapy (FIG. 13C). All live cells from the tumor, spleen, and blood using FlowSOM were analyzed, an unsupervised clustering algorithm, to identify different cell populations (meta-clusters) and mapped these meta-clusters onto UMAP (uniform manifold approximation and projection for dimension reduction) plots (FIG. 5F left). Splenic (FIG. 5F right), tumor (FIG. 5F right), and blood (FIG. 14D) UMAP plots revealed striking differences in the relative proportion of the different immune subsets in response to the different treatments. In the spleen (FIG. 14A and FIG. 14C) and blood (FIG. 14B and FIG. 14E), Odronextamab monotherapy was sufficient to mediate a significant depletion of B cells in comparison to isotype and REGN5837 monotherapy treatment. Importantly, the combinatorial treatment depleted splenic B cells to a similar extent as Odronextamab monotherapy with a trend towards expanding splenic (FIGS. 14A and C) and blood (FIGS. 14B and E) T cell populations in comparison to isotype treated animals. In addition, within the tumor, the combination of Odronextamab and REGN5837 treatment resulted in a significant reduction in the frequency of tumor cells (FIG. 13D left) with a concomitant expansion of intratumoral T cells (FIG. 13D right) in comparison to Odronextamab monotherapy.

Enumeration of the absolute counts of tumor cells revealed a significant suppression of tumor growth in response to Odronextamab or combinatorial treatment in comparison to isotype treated animals (FIG. 5G below). While there was not a significant difference at this time point in tumor volume (FIG. 13C) or absolute count of WSU-DLCL2 cells between combinatorial or Odronextamab treatment (FIG. 5G below), there was a significant expansion of CD4⁺ and CD8⁺ T cells in response to combination treatment (FIG. 5G top) when compared to either Odronextamab or REGN5837 monotherapy. Examining the induction of memory subsets revealed that treatment with Odronextamab or combination treatment significantly drove the expansion of effector memory (EM) CD8⁺ (FIG. 13E) and CD4⁺ (FIG. 13F) T cells with over 90% acquiring an EM phenotype. While there was a slight trend towards increased EM formation in response to combinatorial treatment in comparison to Odronextamab (CD8⁺ T cells: 96% vs 91%; CD4⁺ T cells: (93% vs 90%), there was however, a significant increase in the density of effector and central memory CD8⁺ (FIG. 13G) and CD4⁺ (FIG. 13H) T cells due to the overall increase in total intratumoral T cells.

In order to gain a better understanding of how the different treatment were modulating intratumoral T cell phenotypes, intratumoral CD4⁺ and CD8⁺ T cell populations were analyzed with FlowSOM and identified 11 meta-clusters (FIG. 5H left). UMAP plots revealed that combination treatment resulted in a striking enrichment for metacluster 1 and suppression of metacluster 3 within intratumoral CD8⁺ T cells, and a skewing towards metacluster 11 and away from metacluster 10 within intratumoral CD4⁺ T cells (FIG. 5H and FIG. 5J). Metacluster 1 represents a population of CD8⁺ T cells that has a reprogrammable)(CD38^{lo}CD101^{lo}) phenotype, or a reversible state of dysfunction that has previously been described in the literature while metacluster 3 represents a dysfunctional population of CD8 T cells (PD1^{hi}CD28⁺Ki67⁺) that is highly proliferative but associated with a lack of effector cytokine production and tumor progression (FIG. 5I). Metacluster 11 is a population of PD-1⁺CD38^{lo} CD4⁺ T cells which is reported to exhibit better tumor control than CD38^{hi} CD4⁺ T cells in metacluster 10 due to the maintenance of stemness and effector function and the prevention of metabolic exhaustion (FIG. 5I). Therefore, these data demonstrated that not only does costimulation with the CD22×CD28 bispecific antibody in combination with Odronextamab expand intratumoral T cells, but also prevents the induction of dysfunctional T cells leading to the enhancement of anti-tumor immunity.

While REGN5837 Monotherapy does not Activate T Cells in Cynomolgus Monkey Studies, the CD28 Bispecific Antibody Augments Odronextamab-Mediated T Cell Activation.

To evaluate the tolerability of REGN5837 alone or in combination with Odronextamab, a study was performed in cynomolgus monkeys. Three monkeys per group received a single intravenous slow bolus of REGN5837 (10 mpk) alone, Odronextamab alone (0.001, 0.01, 0.1, or 1 mpk), or REGN5837 (1 or 10 mpk) followed by Odronextamab (0.001, 0.01, or 0.1 mpk). Assessment of tolerability and pharmacologic activity during the in-life phase of the study included body weight, clinical observations, veterinary physical examinations (which included assessments of heart rate, body temperature, and respiratory rates), neuromuscular/musculoskeletal observations, clinical pathology (hematology, serum chemistry [including C-reactive protein], coagulation and urinalysis). In addition, blood samples were collected for cytokine profiling and immunophenotyping analysis by flow cytometry at 5 hrs, 1 day, 4 days, and upon completion of the study at day 8 post dosing. At necropsy, full gross and microscopic evaluations were conducted. Single or repeated intravenous administration of REGN5837 or Odronextamab as monotherapy or in combination were not associated with any moribundity or early deaths. In addition, there were no adverse test article-related findings in clinical observations, including no signs of cytokine release syndrome (CRS) (e.g. emesis or fecal changes).

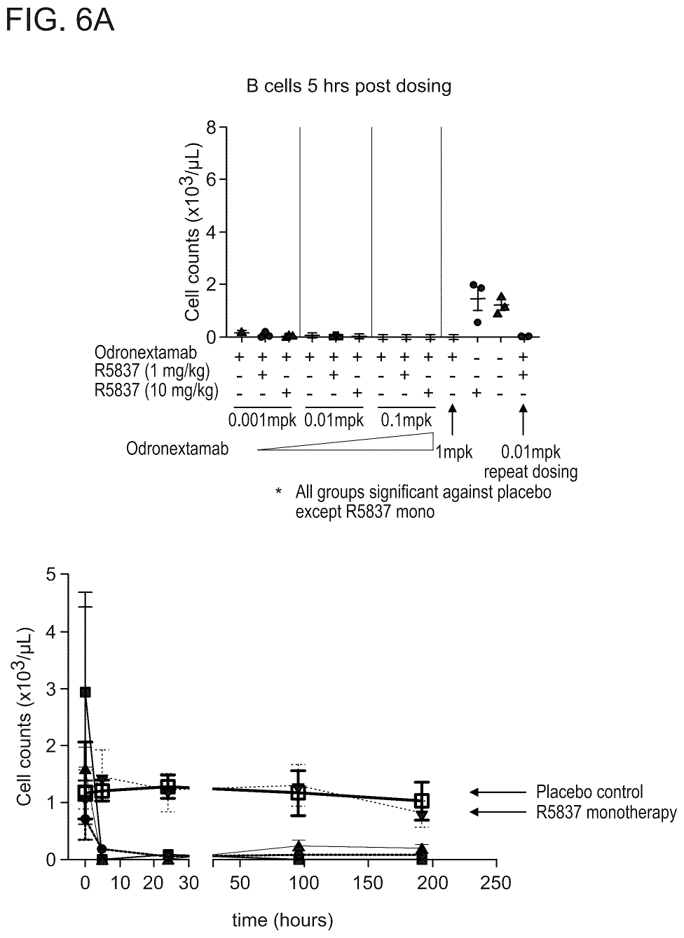

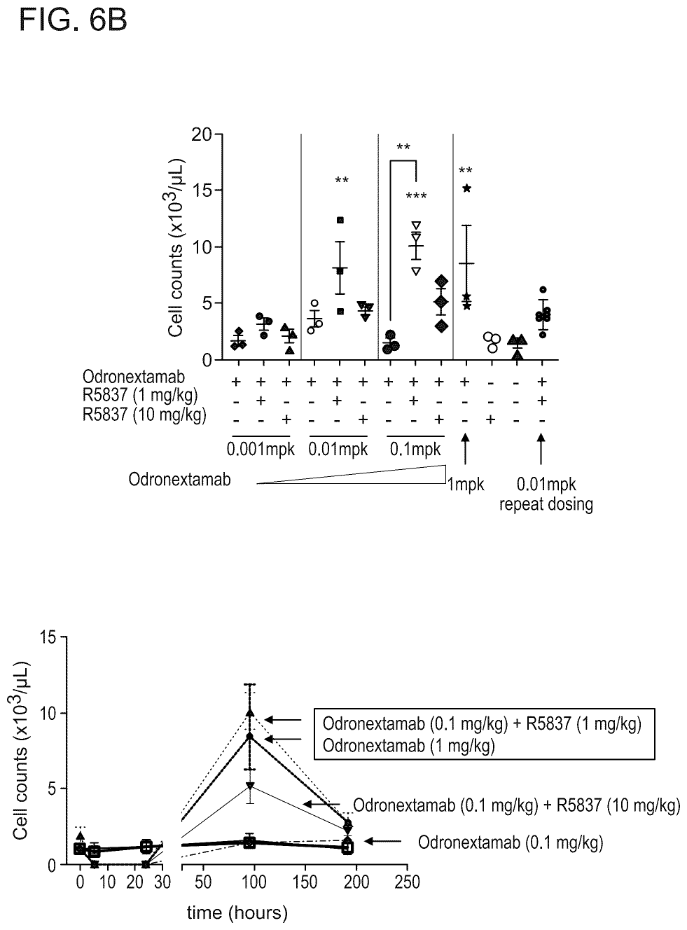

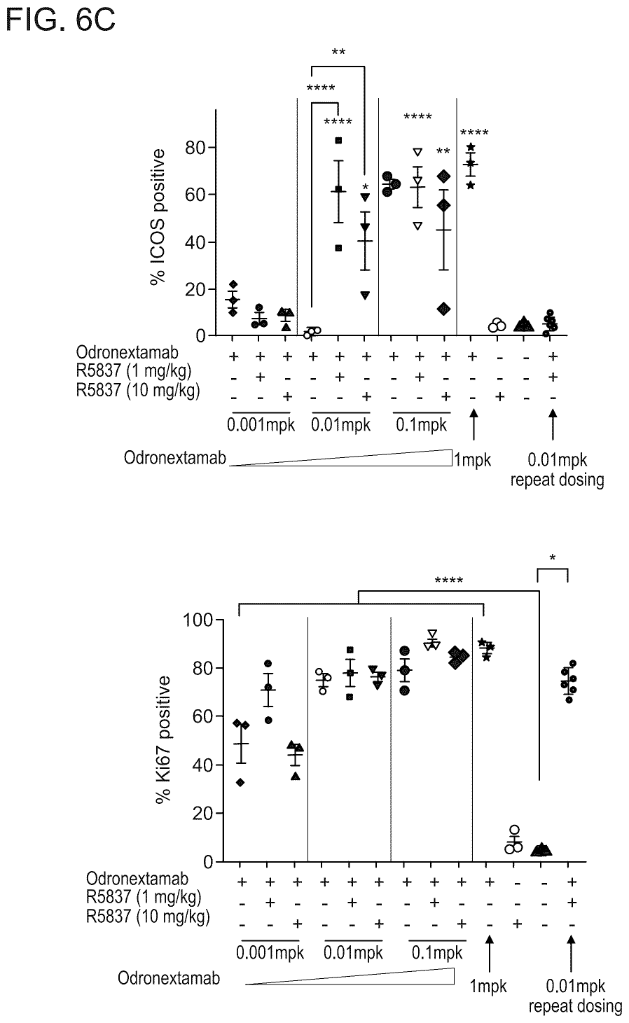

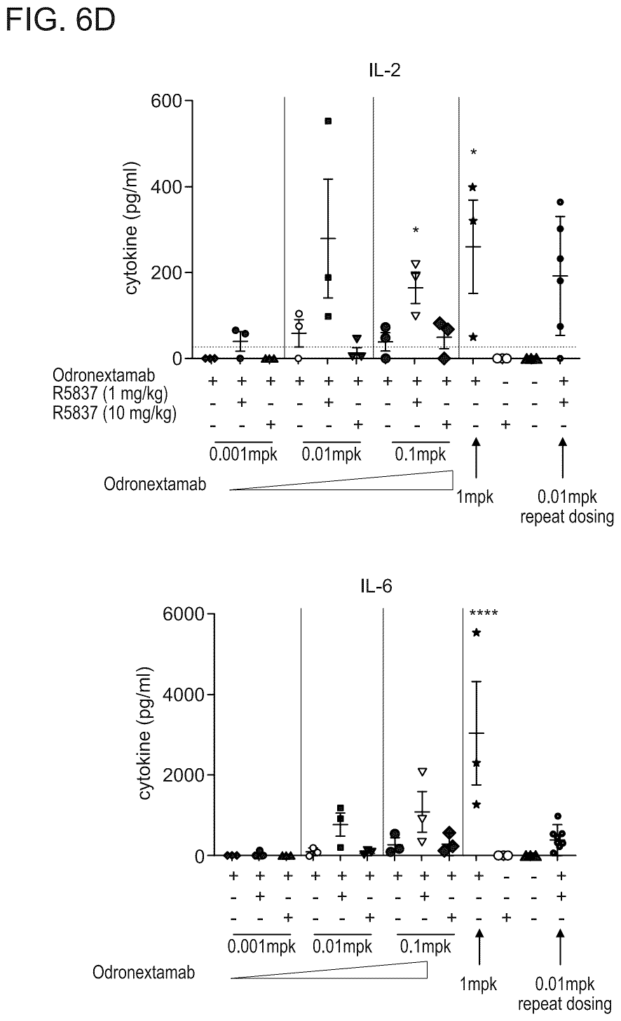

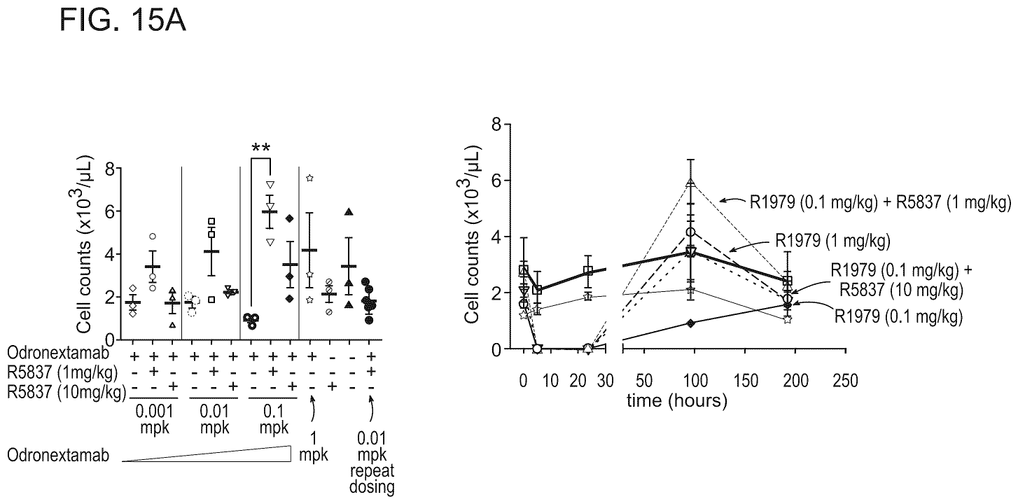

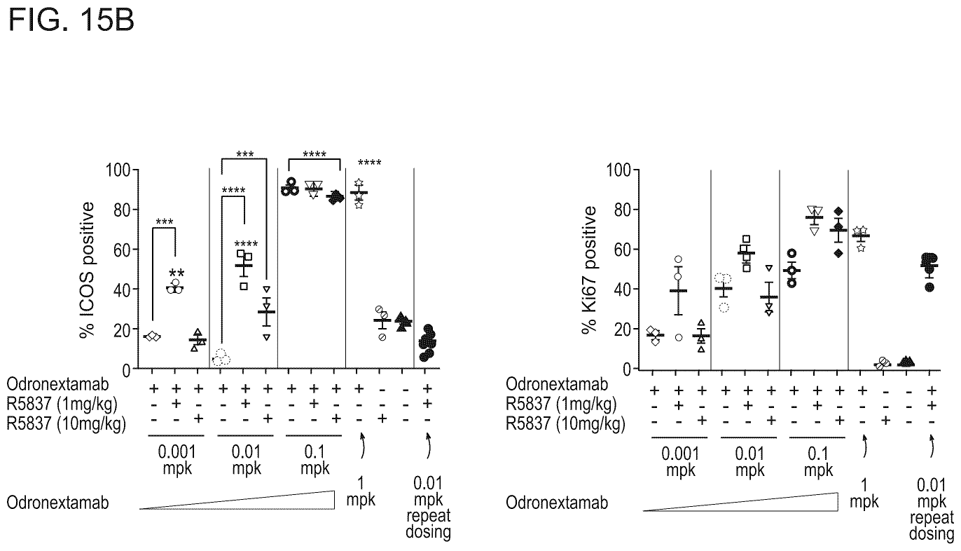

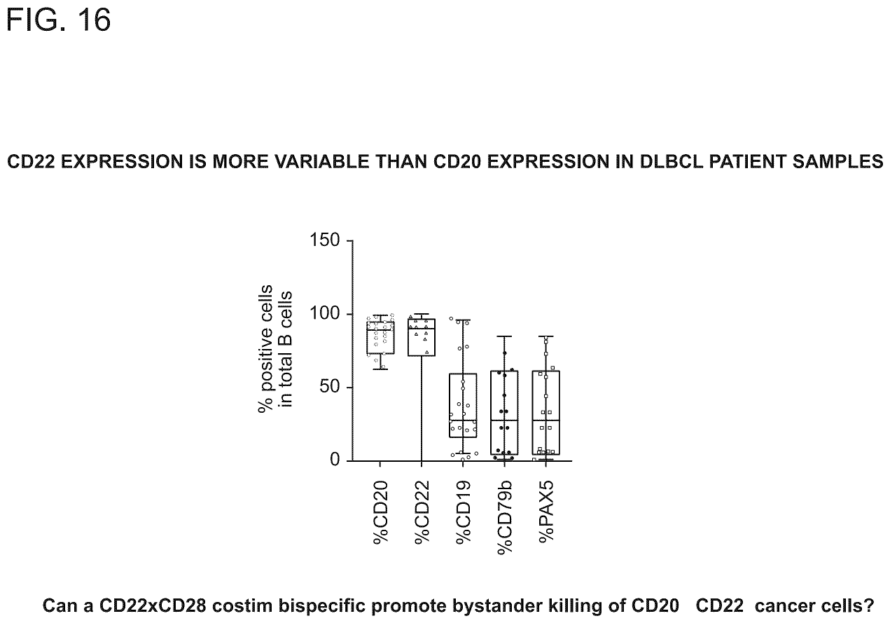

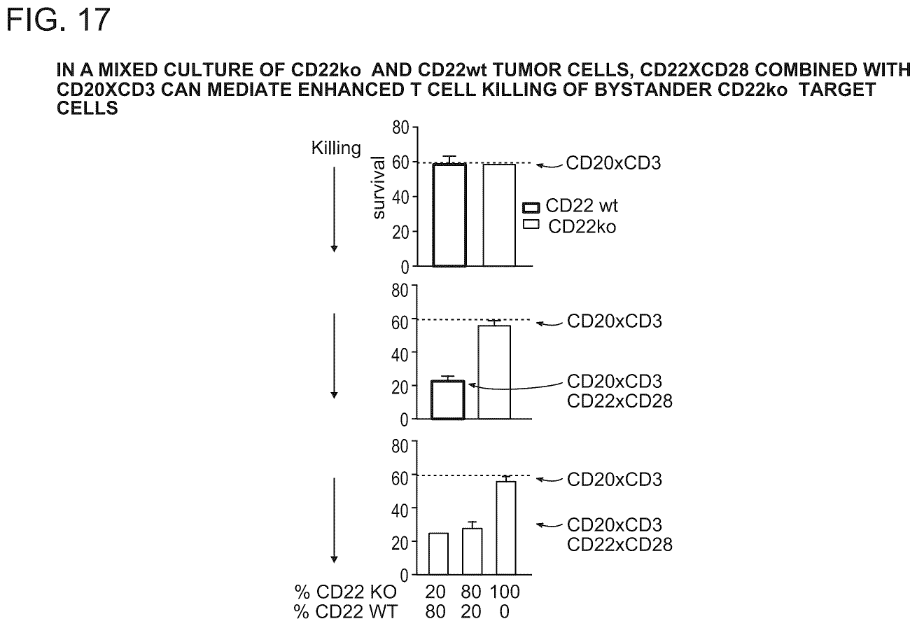

As previously described for other CD28-based costimulatory bispecific antibodies, treatment with REGN5837 as a single agent did not induce peripheral CD4⁺ or CD8⁺ T cell expansion or activation (FIG. 6B, C and FIG. 15A-B), consistent with in vitro data demonstrating that the CD28 bispecific antibody does not induce T cell activation in the absence of signal 1 (FIG. 9A). Additionally, REGN5837 monotherapy had no effect on B cell numbers in the periphery (FIG. 6A). However, consistent with previously published data for CD3 bispecific antibodies, Odronextamab induced an initial lymphocyte margination followed by a trend towards expansion and activation of CD4⁺ (FIG. 15A, B) and CD8⁺ (FIG. 6B and FIG. 6C) T cells as shown by upregulation of ICOS and induction of proliferation. Concomitant with T cell activation was the sustained depletion of peripheral B cells at all evaluated concentrations of Odronextamab (FIG. 6A). Addition of REGN5837 to Odronextamab efficiently depleted peripheral B cells similarly to Odronextamab alone. Of note, combination with REGN5837 at 1 mpk significantly expanded and activated peripheral CD8⁺ (FIGS. 6B and 6C) and CD4⁺ T (FIGS. 15A and 15B) cells in comparison to Odronextamab monotherapy and placebo control. While combination therapy with REGN5837 at 10 mpk also induced a trend towards enhanced Odronextamab-mediated T cell activation, the addition of the costimulatory antibody at this concentration did not significantly augment T cell activity to the same extent as REGN5837 dosed at 1 mpk. This may be due to one-armed binding of the CD28 bispecific at high concentrations, resulting in a bell-shaped curve when examining parameters of T cell activation.

Odronextamab monotherapy resulted in dose-dependent increases in serum cytokines (FIG. 6D) which correlated with previous observations from additional tumor targeted CD3 bispecific antibodies. The combination of Odronextamab (at 0.1 mpk) with REGN5837 (at 1 mpk) significantly increased IL-2 production in comparison to placebo, with a trend towards increased serum IL-6, reflecting the increased T cell activation observed with combinatorial treatment (FIG. 6C and FIG. 15B). Of interest, combination of Odronextamab at 0.1 or 0.01 mpk with REGN5837 (1 mpk) did not induce IL-6 as strongly as Odronextamab dosed alone at a 1 mpk; however, the combinatorial treatment expanded and activated CD4⁺ and CD8⁺ T cells to a similar extent as Odronextamab monotherapy at 1 mpk (FIG. 6B and FIG. 15A). Overall, these results demonstrate that the addition of REGN5837 to Odronextamab enhanced T cell activation in cynomolgus animals by inducing increased serum cytokines and expanding peripheral T cells.

### Conclusion

Here it is demonstrated that REGN5837 can potentiate Odronextamab mediated activity by promoting cytotoxicity of DLBCL and other NHL cell lines in vitro and by potently enhancing anti-tumor activity against DLBCL tumors that cannot be cleared by CD3 bispecific antibody treatment. While Odronextamab as a single agent suppressed WSU-DLCL2 tumor growth, only combinatorial treatment with REGN5837 resulted in curative responses and enhanced overall survival in preclinical in vivo models. In addition, the combination of REGN5837 and Odronextamab not only maintained cytotoxicity against CD20 expressing cells, but also resulted in a significant expansion of intratumoral T cells, with an enhanced induction of effector memory cells in comparison to Odronextamab monotherapy. Further characterization of the intratumoral immune compartment from HIS mice revealed that the addition of CD28 costimulation to Odronextamab skewed the CD8⁺ T cell population to a reprogrammable phenotype) (CD38^{lo}CD101^{lo}), or a plastic dysfunctional state that under the right conditions, can be reversed, allowing the re-invigorated T cells to produce high levels of pro-inflammatory cytokines and mediate anti-tumor immunity. This expansion was accompanied by a concomitant decrease in the induction of dysfunctional CD28⁺Ki67⁺CD8⁺ T cells, an overactivated population which has been previously described to be lacking in effector function and amplified in melanoma and later stages of NSCLC and associated with resistance to PD-1 blockade therapy. Adding REGN5837 also expanded a population of PD-1⁺CD38^{lo} CD4⁺ T cells that are reported to exhibit superior tumor control in comparison to CD38^{hi} T cells due to the maintenance of effector function, promotion of T cell persistence, and metabolic reprogramming being driven by enhanced glutaminolysis. Therefore, this data suggest that REGN5837 not only expands intratumoral T cells, but also prevents the induction of dysfunctional T cells, allowing for the enhancement of anti-tumor immunity.

Toxicology studies in cynomolgus monkeys and analysis of peripheral blood from HIS mice demonstrated that REGN5837 has little activity and does not exhibit toxicity as a monotherapy despite the expression of CD22 on peripheral B cells due to the absence of signal 1. Only when REGN5837 was dosed in combination with Odronextamab, which mediates TCR clustering, was peripheral T cell activation and expansion observed. Of note, in cynomolgus monkey studies, the addition of REGN5837 to low dose Odronextamab was able to mediate similar levels of peripheral T cell expansion as 10-100 fold higher doses of the CD3 bispecific; however, this expansion was associated with a lower induction of serum cytokines compared to the high dose monotherapy treatment with the CD3 bispecific in cynomolgus studies. Therefore, treatment with either the combination or high dose CD3 bispecific antibody was well tolerated without any signs of cytokine release syndrome (CRS) in the animals.

Odronextamab is currently being evaluated in Phase 1 and Phase 2 clinical trials for the treatment of r/r B cell NHL, where the patient population has been heavily pre-treated and has failed at least 2 prior lines of treatment. Results from early Odronextamab clinical trials reveal encouraging activity with an ORR of 92.9% and a complete response rate (CR) of 75.0% for refractory follicular lymphoma patients receiving at least 5 mg of Odronextamab weekly. Heavily pretreated r/r DLBCL patients dosed at >=80 mg and not given prior CAR T therapy have ORR and CR rates of 60%, while patients refractory to CAR T therapy have an ORR of 33.3% and a CR rate of 23.8%. Overall, the safety profile appears manageable and consistent with the class of CD20×CD3 bispecifics. While promising, these data reveal that there is still room for improvement for the treatment of r/r DLBCL patients, especially in the post CAR T therapy setting. Collectively, this data shows that REGN5837, a novel CD22×CD28 bispecific antibody, could enhance the anti-tumor activity of Odronextamab and provide support for the investigation of REGN5837 with Odronextamab in hard-to-treat patients with aggressive lymphomas.

### Example 2: Material and Methods for Example 1

### Study Design

This study demonstrated that a tumor-targeted CD28 bispecific antibody could potentiate Odronextamab mediated T cell activation to enhance anti-tumor activity. Control and experimental treatments were administrated to age- and sex-matched mice. Sample sizes were chosen empirically to ensure adequate statistical power and were in the line with field standards for the techniques used in the study. All animals were randomized to different treatment groups based on tumor volumes prior to start of treatment and investigators were blinded to all treatment groups. The number of experimental replicates is indicated in the figure legends.

### Cell Lines

For the WSU-DLCL2 studies, the DLBCL cell line was obtained from DSMZ (ACC 575) and maintained in RPMI-1640 with 10% FBS (Seradigm) supplemented with penicillin, streptomycin, glutamine, and 1 mM HEPES (Gibco). A CRISPR-edited CD22 deficient line was generated by electroporation of Cas9 ribonucleoprotein (RNP) with TrueCut™ Cas9 Protein v2 (Invitrogen) and a High Scoring TrueGuide™ Synthetic sgRNA targeting human CD22 (Invitrogen guide RNA: CCGGTGCACCTCAATGACAG) using the Neon™ Transfection System 100 μL Kit (Invitrogen). CD22 deficient cells were bulk sorted 96 hours post electroporation.

For the NALM6-luc tumor experiments, the NALM6 cell line (DSMZ: ACC 128) was modified with the EF1a-Luciferase-2A-GFP-Puro lentivirus (GenTarget) in order to image tumor cell growth in vivo. The cell line was maintained in RPMI with 10% FBS supplemented with PSG (penicillin, streptomycin, and glutamine) and puromycin selection (1 ug/ml).

### Animal Studies

All procedures were carried out in accordance with the Guide for the Care and Use of Laboratory Animals of the NIH. The protocols were approved by the Regeneron Pharmaceuticals Institutional Animal Care and Use Committee (IACUC) and all animals were maintained under pathogen-free conditions.

For the NSG experiments, WSU-DLCL2 cells (3×10⁶ cells) were collected and mixed with 5×10⁵ PBMCs (ReachBio) and resuspended in a 1:1 mixture of PBS and GFR Matrigel (Corning). Female NSG mice (Jackson Laboratory) were subcutaneously injected with the cell mixture in the right flank. For the human immune system reconstituted experiments, WSU-DLCL2 cells (3×10⁶ cells) were implanted subcutaneously in SIRPA^{h/h} TPO^{h/m} Rag2^{-/-}Il2rg^{-/-} mice that were engrafted with fetal liver CD34^{+ cells; animals were segregated to have similar distribution of fetal liver donors, human immune cell engraftment frequency, and sex for each treatment group. Mice were randomized to receive blinded treatments of either isotype controls (EGFRV}3×CD3 or MUC16×CD28) or test articles (REGN5837, Odronextamab) which were administered as monotherapy or in combination by intraperitoneal injection at specified concentrations on day 1, day 8, and day 15 post implantation for prophylactic treatment, and on d8, d15, and d22 for therapeutic treatment. Tumor growth was monitored over time using digital caliper (VWR) measurements of X and Y diameter (perpendicular measurements of length and width). Tumor volume was calculated (X*Y*(X/2) where X is the shorter diameter). Mice were euthanized when the tumor reached a designated tumor end-point (tumor diameter>20 mm, or tumor ulceration). This designated endpoint is in accordance with IACUC standards.

For the NALM6-luc tumor experiments, female immunodeficient NSG mice (Jackson Laboratories) were engrafted with 4×10⁶ human PBMCs (ReachBio) and animals that were successfully engrafted were injected intravenously with 5×10⁶ cells of NALM6-luc cells 12 days post PBMC engraftment. Mice were randomized to receive blinded treatments of either isotype controls (EGFRV3×CD3 or MUC16×CD28) or test articles (REGN5837, Odronextamab) which were administered as monotherapy or in combination by intraperitoneal injection at specified concentrations on day 8, day 15, and day 22 post implantation. NALM6-luc implanted mice were imaged twice a week using the IVIS Spectrum (Perkin Elmer) after being injected intraperitoneally with the substrate luciferin (PerkinElmer). Bioluminescence (total flux) was quantified using Living Image software. Experiment was ended when mice began exhibiting signs of GVHD (weight loss>=20%) in accordance with IACUC standards.

### Measurement of Serum Cytokines in Mice

At the indicated time points, blood was collected from submandibular vein into microtainer serum tubes (BD 365967). Cytokine concentrations were analyzed using V-plex Human ProInflammatory-10 Plex kit following the manufacturer's instructions (Meso Scale Diagnostics).

### Flow Cytometry

For immunophenotyping experiments, tumors, spleens, blood were harvested on indicated days. Single-cell suspensions were prepared, and live/dead cell discrimination was performed using Live/Dead Fixable Blue Dead Cell Staining Kit (Thermo Fisher Scientific). To quantify cell numbers in tissues, a fixed number of CountBright absolute counting beads (Thermo Fisher Scientific) were added to each sample before acquiring. Samples were acquired on a Symphony (BD Biosciences) and analyzed using FlowJo (TreeStar), or OMIQ.

To identify T cell clusters automatically on the basis of selected markers, FlowSOM from OMIQ was run on samples acquired on the Symphony (BD Biosciences). Analyses were run on equal numbers of events per sample. The range in events was determined by the sample with the fewest cells. FlowSOM clustering of intratumoral CD8⁺ T cells in Omiq.ai was run on selected parameters (activation/dysfunction and memory markers) with default settings. In order to visualize the clusters identified by FlowSOM, UMAP, a high dimensional reduction method, was run on all samples within Omiq.ai. Samples from each treatment group were concatenated and FlowSOM clusters overlaid upon the UMAP plots.

### Wet Plate Coating Assay

Antibodies diluted to 10 μg/ml in PBS were wet-coated onto polypropylene plates overnight. Diluted antibody (100 μl) was added in triplicate to a 96-well polypropylene assay plate. Plates were stored overnight at 4° C. to allow adsorption of the antibodies. and the plates washed twice with PBS prior to use in the proliferation assay. PBMCs were isolated from leukocyte-enriched peripheral blood (New York Blood Center) obtained from four healthy individual donors. The PBMCs from each donor were resuspended in RPMI medium (Irvine Scientific) containing 10% human AB serum (GemCell) and penicillin/streptomycin/glutamine (100 units/ml, 100 μg/ml, 292 μg/ml, respectively, Gibco) and then added to the 96-well assay plates at 100,000 cells/well in a final volume of 200 μl/well. The assay plates were then incubated at 37° C.+5% CO₂ for 54 hours. At 54 hours, the assay plates were centrifuged and 100 μl of supernatant was removed for cytokine analysis. Cytokine concentrations for IFNg, IL1B, IL2, IL4, IL6, IL8, IL10, IL13, and TNFa were determined using the V-PLEX Proinflammatory Panel 1 Human Kit according to the manufacturer's instructions. Mean and range of concentrations (pg/mL) obtained from 4 donors were plotted, with individual data points representing average concentrations for each individual donor obtained from assays performed in triplicate wells.

To evaluate proliferation, 100 μl of 1 mCi/ml tritiated thymidine (Perkin Elmer) was added to each well and incubated at 37° C.+5% CO₂ for 18 additional hours. Each 96-well assay plate was harvested using a Filtermate Harvester (Perkin Elmer) and analyzed on a TopCount scintillation counter (Perkin Elmer). The amount of radioactivity was measured as counts per minute (CPM) per well and was proportional to the number of proliferating cells. CPM values are presented with four data points representing mean counts for each individual donor obtained from assays performed in triplicate wells.

### In Vitro T Cell Activation Assay

Previously frozen human CD3⁺ T-cells isolated from healthy donor leukocyte packs via density gradient centrifugation using 50 mL SepMate™ tubes were thawed the day of the assay in stimulation media (X-VIVO 15 cell culture media supplemented with 10% FBS, HEPES, NaPyr, NEAA, and 0.01 mM BME) containing 50 U/ml benzonase nuclease and plated into 96-well round bottom plates at a concentration of 1×10⁵ cells/well. NALM6 cells were treated with 10 ug/mL of Mitomycin C in primary stimulation media at a concentration of 10×10⁶ cells/mL. After incubation for 1 hour at 37° C., 5% CO₂, mitomycin C-treated cells were washed 3 times with D-PBS containing 2% FBS and added to the wells containing CD3⁺ T-cells at a final concentration of 5×10⁴ cells per well. To all wells, irrelevant hIgG1 mAb was added (100 nM/well) to block Fc receptors. A fixed concentration of Odronextamab or non-binding isotype control at 500 pM with a dose titration of REGN5837 or a non-binding isotype control from 3.1 pM to 200 nM was added to the wells. Plates were incubated for 72 hours at 37° C. and 5% CO₂, at which time 50 μl of culture supernatant was collected. 5 μl of this collected supernatant was tested according to the manufacturer's protocol for human IL-2 (AL221F) AlphaLISA assay (Perkin Elmer). The measurements were acquired on an Envision multilabel plate reader (Perkin Elmer). A standard curve of known concentrations was generated to extrapolate the concentration of IL-2 generated in assay wells. To assess T cell proliferation, medium was supplemented with a final concentration of 1.25 μM [³H] thymidine (Perkin Elmer) and the cells were incubated at 37° C., 5% CO₂ for 16 hours. Plates were harvested using Microbeta Filermat-96 Cell Harvester (Perkin Elmer), 30 μL MicroScint-20 (Perkin Elmer) was added, and [³H] thymidine incorporation was measured using Microplate Scintillation Counter TopCount NXT (Perkin Elmer). All serial dilutions were tested in triplicate. The EC₅₀ values of the antibodies were determined from a four-parameter logistic equation over a 10-point dose-response curve using GraphPad Prism software.

### In Vitro Cytotoxicity Assay

Human PBMCs were thawed and plated in complete media (RPMI cell culture media supplemented with 10% FBS, penicillin-streptomycin-glutamine) at 1×10⁶ cells/mL and incubated overnight at 37° C. in order to enrich for lymphocytes by depleting adherent cells such as macrophages, dendritic cells, and some monocytes. The following day, PBMC were harvested and labeled with 1 μM of Violet Cell Tracker fluorescent tracking dye. WSU-DLCL2 cells were labeled with 1 μM of the fluorescent dye Vybrant CFDA-SE. After labeling, 5,000 labeled target cells were plated in round-bottom 96 well plates at a 1:5 ratio with labeled PBMC. Serial dilutions of R5837 were combined with serial dilutions of R1979 and added to labeled target and effector cells, and the plates incubated were for 72 hours at 37° C. After incubation, the cells were washed and stained with LIVE/DEAD stain in PBS, followed by staining with a cocktail of fluorophore-labeled antibodies to CD2, CD4, CD8, and CD25 for analysis of surviving target cells, T cell activation and proliferation. Counting beads (20 μL per well) were added immediately before sample analysis on a BD Celesta flow cytometer.

Target cell killing was assessed by calculating the number of live, CFDA-SE labeled target cells/well by normalizing to the number of beads collected/well. Percent viability was normalized to the number of living target cells in the control condition (target cells in the presence of PBMC only). T cell activation was assessed by reporting the % CD25 on CD4+ and CD8+ T cells. T cell proliferation was assessed by reporting the percentage of cells that had decreased MFI of the Violet Cell Tracker dye.

Supernatants from this assay for the were collected for analysis of cytokine levels. Concentrations of IL 17a, IFNγ, TNFα, IL-10, IL-6, IL-4, and IL-2 were analyzed using a Cytometric Bead Array (CBA) kit following the manufacturer's instructions. Briefly, 1/15 dilutions of the supernatants along with the standard samples were incubated in a 96-well assay plate with the cytokine-specific bead array for 3 hours at room temperature. Following the incubation, the samples were washed twice and analyzed by flow cytometry on BD FACS Canto II. Cytokine levels were interpolated from the MFI of the kit standards and reported as pg/mL.

The EC50 values of the antibodies were determined from a four-parameter logistic equation over a 9-point dose-response curve using GraphPad Prism software. Maximum responses for percent cytotoxicity, T cell activation, proliferation and cytokine release was taken as the plateau value generated by the Prism curve fit. Fold change in EC50 was calculated as EC50_{NoR5837}/EC50_{[M]R5837} and fold change in maximum cytokine release was calculated as Max_{[M]R5837}/Max_{NoR5837}.

### Chromogenic IHC

Chromogenic IHC assays were performed on the Ventana Discovery ULTRA platform on formalin-fixed paraffin-embedded DLBCL patient samples (Tristar). Antigen retrieval was performed using Tris-EDTA pH9 buffer and slides were incubated with the primary antibodies listed below. The OptiView DAB detection system was used and for some markers an additional amplification step was necessary to visualize the signal. Slides were counterstained with Novolink Hematoxylin (Leica Microsystems, Inc.) and cover slipped (Corning Coverglass, #1) with Cytoseal 60 (Thermo Scientific/Richard Allen Scientific). Once mounting medium had dried completely, slides were scanned at 40× on the Leica Aperio AT2 scanner. Images were analyzed using Indica HALO software.

TABLE 8

| | | | | Catalogue | Detection |
|---|---|---|---|---|---|
| Target | Clone | | Vendor | number | method |
| CD20 | SP32 | Rabbit | ABCAM | ab64088 | OptiView DAB |
| | | Monoclonal | | | |
| CD22 | SP104 | Rabbit | Roche | 06391117001 | OptiView DAB |
| | | Monoclonal | | | |
| CD28 | EPR22076 | Rabbit | ABCAM | ab243228 | OptiView DAB |
| | | Monoclonal | | | with |
| | | | | | Amplification |
| CTLA4 | CAL49 | Rabbit | ABCAM | ab2377712 | OptiView DAB |
| | | Monoclonal | | | with |
| | | | | | Amplification |
| CD80 | 37711 | Rabbit | R&D | MAB140- | OptiView DAB |
| | | Monoclonal | Systems | 500 | with |
| | | | | | Amplification |
| CD86 | E2G8P | Rabbit | Cell | 91882 | OptiView DAB |
| | | Monoclonal | Signaling | | with |
| | | | | | Amplification |

### Multiplex IHC

A fully automated multiplex immunohistochemistry assay was performed on the Ventana Discovery ULTRA platform (Ventana Medical Systems, Tucson, AZ). Five rounds of sequential primary antibody and secondary-Horse Radish Peroxidase-conjugated antibody applications were performed. Heat denaturation between each step to completely remove the bound primary and secondary antibody was performed to eliminate downstream cross-reactivity. This allowed primary antibodies raised in the same species to be used. The fluorescent dyes used were carefully selected to ensure spectral separation and provide optimal staining. The combination and order of application of the primary antibody and tyramide-fluorophore was optimized to ensure that both the epitope and fluorophore could withstand the repeated heat denaturation steps. Optimal concentrations of each antibody were determined, and they were applied in the following sequence and detected with the indicated fluorophore.

TABLE 9

| | | | | Catalogue | Roche | Catalogue |
|---|---|---|---|---|---|---|
| Target | Clone | | Vendor | number | Fluorophore | number |
| CD20 | SP32 | Rabbit | ABCAM | ab64088 | DISCOVERY | 7988168001 |
| | | Monoclonal | | | Rhodamine 6G | |
| CD22 | SP104 | Rabbit | ABCAM | ab240404 | DISCOVERY | 7988192001 |
| | | Monoclonal | | | DCC | |
| CD19 | SP291 | Rabbit | ABCAM | ab237772 | DISCOVERY | 7988176001 |
| | | Monoclonal | | | Red 610 | |
| CD79b | SP240 | Rabbit | ABCAM | ab245745 | DISCOVERY | 7551215001 |
| | | Monoclonal | | | Cy5 | |
| PAX5 | SP34 | Rabbit | Roche | 05552729001 | DISCOVERY | 7988150001 |
| | | Monoclonal | | | FAM | |
| CD28 | EPR22076 | Rabbit | ABCAM | ab243228 | DISCOVERY | 7988168001 |
| | | Monoclonal | | | Rhodamine 6G | |
| CD3 | SP162 | Rabbit | ABCAM | ab135372 | DISCOVERY | 7988192001 |
| | | Monoclonal | | | DCC | |
| CD8 | SP239 | Rabbit | ABCAM | ab178089 | DISCOVERY | 7988176001 |
| | | Monoclonal | | | Red 610 | |
| Ki67 | EPR3610 | Rabbit | ABCAM | ab92742 | DISCOVERY | 7988150001 |
| | | Monoclonal | | | FAM | |

Following staining, the tissue was counter-stained and cover slipped with Invitrogen ProLong Gold Antifade Mountant with NucBlue. Whole slide imaging was performed on the Zeiss Axioscan which was equipped with a Colibri light source and appropriate filters for visualizing these specific fluorophores. Quantitative image analysis was preformed using the HALO Indica Labs Hyperplex module (IndicaLabs, Albuquerque, NM). Numbers of positive cells for each immune subset and density in the entire tumor area were measured.

### Cynomolgus Toxicology Study

The cynomolgus monkey studies were conducted at facilities accredited by the Association for Assessment and Accreditation of Laboratory Animal Care and the Animal Welfare Assurance issued by the Office of Laboratory Animal Welfare, registered with the U.S. Department of Agriculture and IACUC. The study was carried out at Altasciences Preclinical Seattle (formerly SNBL USA) using male cynomolgus monkeys (Macaca fascicularis) (three animals per group). Subjects received a single dose of each test article via intravenous bolus (REGN5837) or intravenous infusion (Odronextamab via ˜30 min infusion). Combination treatment was administered sequentially with Odronextamab infusion starting 5-10 min post administration of REGN5837. Assessments of treatment related effects included body weight measurements, clinical observations, veterinary physical examinations (which included assessments of heart rate, body temperature, and respiratory rates), neuromuscular/musculoskeletal observations, clinical pathology (hematology, blood and/or tissue samples were collected for cytokine analysis, immunophenotyping analysis, histopathology, and toxicokinetic evaluation. For peripheral blood flow cytometry, blood was collected into potassium EDTA tubes; lysed; stained with CD3, CD4, CD8, CD14, CD16, CD20, CD28, and Ki67 (BD Biosciences), and CD278 (BioLegend); and analyzed with FACSCanto II flow cytometer. For cytokine analysis, blood was collected into serum separator tubes with anticoagulant. Serum was separated via centrifugation at 1000 g to 2000 g at 4° C. for 10 to 15 min and analyzed using the MSD U-Plex platform (IL-1β, IL-2, IL-6, MCP-1, and TNF-α, and IFN-γ.).

### Statistical Analysis

Sample sizes were chosen empirically to ensure adequate statistical power and were in the line with standards for the techniques used in the study. Statistical significance was determined with one-way and two-way analysis of variance (ANOVA) and the Kaplan-Meier method with long-rank test to compare survival between groups. Graph generation and statistical analyses were performed using GraphPad Prism (version 8).

### Example 3: REGN5837 Enhances Cytotoxicity Mediated by Human T Cells Activated with the CD20×CD3 Bispecific Antibody REGN1979 Against B Cell Lymphoma Cells that do or do not Express Surface CD22

T cells were isolated from freshly thawed PBMC using the EasySep Human T-Cell Isolation kit and used immediately. WSU-DLCL2/CD22^{WT} cells were labeled with 1 μM of the fluorescent dye Vybrant CFDA-SE. WSU-DLCL2/CD22K° cells were labeled with 1 μM of the fluorescent dye CellTrace Far Red. Labeled WSU-DLCL2/CD22^{WT} and WSU-DLCL2/CD22^{KO} target cells were plated at different ratios (0:100, 20:80, 40:60, 60:40, 80:20, 100:0) in round-bottom 96 well plates, and unlabeled T cells were added at a final effector to target ratio of 5:1. Target and effector cells were incubated with 5 pM CD20×CD3 (REGN1979) alone, or with different concentrations of REGN5837 (16.7 nM, 2.77 nM, 463 pM), and the plates were incubated for 72 hours at 37° C.

After incubation, the cells were washed and stained with LIVE/DEAD stain in PBS, followed by staining with a cocktail of fluorophore-labeled antibodies to CD4, CD8, and CD25 for analysis of T cell activation. Counting beads (20 μL per well) were added immediately before sample analysis on a BD Celesta flow cytometer.

WSU-DLCL2/CD22^{WT} killing was assessed by calculating the number of live, CFDA-SE labeled target cells/well by normalizing to the number of beads collected/well. WSU-DLCL2/CD22^{KO} killing cell killing was assessed by calculating the number of live, Far-Red labeled target cells/well by normalizing to the number of beads collected/well. Percent viability was normalized to the number of living target cells in the control condition (target cells in the presence of effector cells only). T cell activation was assessed by reporting the MFI of CD25 on CD4+ and CD8+ T cells.

### Results Summary:

The capacity of REGN5837 to enhance cytotoxicity mediated by human T cells activated with the CD20×CD3 bispecific antibody REGN1979 against B cell lymphoma cells that do or do not express surface CD22 was evaluated using flow cytometry. Additionally, T cell activation, as measured by upregulation of CD25 on T cells, was assessed.
5 pM REGN1979 activated and directed human T cells to kill WSU-DLCL2/CD22^{WT} and WSU-DLCL2/CD22^{KO} to a similar extent (Table 8). T cell activation in the presence of 5 pM REGN1979 alone was similar regardless of the ratio of CD22^{KO}:CD22^{WT} cells (Table 11).

REGN5837 enhanced the REGN1979 mediated killing of WSU-DLCL2/CD22^{WT} cells in a dose dependent manner; killing of WSU-DLCL2/CD22^{WT} cells was similar regardless of the presence of WSU-DLCL2/CD22^{KO} in the culture (Table 10).
REGN5837 enhanced the REGN1979 mediated killing of WSU-DLCL2/CD22^{KO} cells in cultures that contained 20% or more WSU-DLCL2/CD22^{WT} cells. REGN5837 did not enhance REGN1979 mediated killing of WSU-DLCL2/CD22^{KO} cells in the absence of any WSU-DLCL2/CD22^{WT} cells in the culture (Table 10).

In cultures containing WSU-DLCL2/CD22^{WT} cells, REGN5837 enhanced REGN1979 mediated T cell activation in a dose dependent manner, with greater T cell activation observed when higher ratios of WSU-DLCL2/CD22^{WT} cells were present. REGN5837 did not enhance REGN1979 mediated T cell activation in the absence of any WSU-DLCL2/CD22^{WT} cells in the culture (Table 11).

In summary, REGN5837 co-stimulation increased REGN1979 mediated killing of target cells lacking CD22 expression as long as CD22 expressing cells were present in the culture.

TABLE 10
% WSU-DLCL2/CD22^{KO} and WSU-DLCL2/CD22^{WT} survival

| | | Ratio CD22^{KO}:CD22^{WT} | | | | | |
|---|---|---|---|---|---|---|---|
| | | 100:0 | 80:20 | 60:40 | 40:60 | 20:80 | 0:100 |
| % CD22^{KO} | 5 pM | 58.6 | 60.15 | 61.75 | 60.55 | 59.45 | NA |
| Survival | R1979 | | | | | | |
| | only | | | | | | |
| | +16.7 nM | 45.4 | 26.95 | 19.1 | 19.85 | 20.85 | NA |
| | R5837 | | | | | | |
| | +2.8 nM | 56 | 28.8 | 28.25 | 24.1 | 25.25 | NA |
| | R5837 | | | | | | |
| | +0.46 nM | 60.1 | 37.2 | 33.9 | 31.85 | 29.9 | NA |
| | R5837 | | | | | | |
| % CD22^{WT} | 5 pM | NA | 54.75 | 55.15 | 49.9 | 48.8 | 58.75 |
| Survival | R1979 | | | | | | |
| | only | | | | | | |
| | +16.7 nM | NA | 7.95 | 8.6 | 9.45 | 11.15 | 12.6 |
| | R5837 | | | | | | |
| | +2.8 nM | NA | 13.1 | 12.45 | 12.8 | 16.45 | 22.4 |
| | R5837 | | | | | | |
| | +0.46 nM | NA | 20.6 | 19.1 | 20.15 | 19.3 | 28.5 |
| | R5837 | | | | | | |

TABLE 11
T cell activation (CD25 upregulation)

| | | Ratio CD22^{KO}:CD22^{WT} | | | | | |
|---|---|---|---|---|---|---|---|
| | | 100:0 | 80:20 | 60:40 | 40:60 | 20:80 | 0:100 |
| CD4+ | 5 pM | 4375 | 4306 | 4483 | 4463 | 3902 | 4633 |
| T cell | R1979 | | | | | | |
| acti- | only | | | | | | |
| vation | +16.7 nM | 3154 | 9853 | 12029 | 13583 | 15280 | 17661 |
| (MFI of | R5837 | | | | | | |
| CD25) | +2.8 nM | 4055 | 8275 | 10895 | 13229 | 14398 | 14845 |
| | R5837 | | | | | | |
| | +0.46 nM | 4280 | 7518 | 8522 | 11751 | 11202 | 13365 |
| | R5837 | | | | | | |
| | No Stim | 678 | 654 | 695 | 657 | 634 | 595 |
| CD8+ | 5 pM | 2502 | 2486 | 2644 | 2617 | 2791 | 2837 |
| T cell | R1979 | | | | | | |
| acti- | only | | | | | | |
| vation | +16.7 | 2159 | 4971 | 6668 | 7880 | 8661 | 9875 |
| (MFI of | nM | | | | | | |
| CD25) | R5837 | | | | | | |
| | +2.8 nM | 2510 | 4894 | 6618 | 7916 | 8413 | 9031 |
| | R5837 | | | | | | |
| | +0.46 nM | 3001 | 4254 | 4796 | 6136 | 6310 | 7919 |
| | R5837 | | | | | | |
| | No Stim | 392 | 375 | 401 | 424 | 371 | 342 |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of mediating killing of a tumor cell, inducing killing of tumor cells, or inducing T cell activation against tumor cells in a tumor in a subject, comprising administering to the subject a multispecific antigen binding molecule with a first antigen binding region specific for a target antigen and a second antigen binding region specific for a CD28 protein, wherein the tumor cell does not express or is not predicted to express the target antigen.

2-3. (canceled)

4. A method of treating cancer in a subject with a tumor, the method comprising administering to the subject a multispecific antigen binding molecule with a first antigen binding region specific for a target antigen and a second antigen binding region specific for a CD28 protein, wherein at least a subset of tumor cells in the tumor do not express the target antigen.

5. The method of claim 1, wherein the method further comprises determining that at least the subset of the tumor cells in the tumor do not express the target antigen, optionally wherein tumor cells do not express the target antigen if the expression of the target antigen is below the level of detection or below signal to noise ratio.

6. (canceled)

7. A method of selecting a subject for cancer therapy, the method comprising:
i) determining that the subject comprises a tumor comprising tumor cells that do not express a target antigen; and
ii) administering to the subject a multispecific antigen binding molecule with a first antigen binding region specific for the target antigen and a second antigen binding region specific for a CD28 protein, optionally wherein the tumor cells do not express the target antigen if the expression of the target antigen is below the level of detection or below signal to noise ratio, thereby selecting a subject for cancer therapy.

8. The method of claim 1, wherein the target antigen is:
i) a tumor associated antigen (TAA),
ii) an antigen associated with the tumor microenvironment of the tumor, optionally wherein the antigen associated with the tumor microenvironment is an antigen associated with the tumor stroma, an antigen associated with the extracellular matrix of the tumor, an antigen associated with a blood vessel in the tumor microenvironment, or an antigen associated with a cancer-associated fibroblast; or
iii) an immune antigen.

9. The method of claim 8,
wherein the target antigen is a TTA selected from CD38, EGFR, CD22, MUC16, PSMA, CA9, FOLR1, HER2, and SLAMF7, or
ii) wherein the target antigen is an antigen associated with tumor stroma selected from PSA, CEA, CA-125, CA-19, COL10, FAP, B7H3, LRRC15, and fibronectin-EDB isoform, or
iii) wherein the target antigen is an antigen associated with the extracellular matrix of the tumor selected from nectin, versican (VACN), fibronectin and a carcinoembryonic antigen-related cell adhesion molecule (CEACAM) protein, or
iv) wherein the target antigen is an antigen associated with a cancer associated fibroblast selected from α-smooth muscle actin (α-SMA), fibroblast activation protein (FAP), S100A4, platelet-derived growth factor receptors (PDGFRα/β), vimentin, PDPN, CD70, CD10, GPR77, CD10, CD74, CD146, CAV1, Saa3- and CD49e, or
v) wherein the target antigen is an antigen associated with the blood vessel in the tumor microenvironment selected from DLK1, EphA2, HBB, NG2, NRP1, NRP2, PDGFRβ, PSMA, RGS5, TEM1, VEGFR1 and VEGFR2, or
vi) wherein the immune antigen is an antigen expressed on the surface of an immune cell, and wherein the immune cell is selected from a macrophage, a neutrophil, an eosinophil, a basophil, a mast cell, a monocyte, a dendritic cell, a natural killer cell, a T cell and a B cell, or
vii) wherein the target antigen is an immune antigen selected from any one of the immune antigens listed in Table 4, or
viii) wherein the target antigen is a TTA, and the tumor is a heterogeneous tumor further comprising tumor cells that express the TAA.

10-17. (canceled)

18. The method of claim 1, wherein the tumor comprises immune cells, and wherein the immune cells are B cells and/or CD20-expressing cells.

19. (canceled)

20. The method of claim 1, wherein the tumor cells that do not express the target antigen also do not express CD20.

21. (canceled)

22. The method of claim 1, wherein the tumor microenvironment of the tumor comprises cells that express the target antigen.

23. The method of claim 1, wherein at least 20%, at least 30%, at least 40%, at least 50%, at least 60%, at least 70%, at least 80%, at least 90%, or 100% of the tumor cells in the tumor do not express the target antigen.

24-31. (canceled)

32. The method of claim 1, wherein at least 10%-100% of the tumor cells in the tumor do not express the target antigen.

33-36. (canceled)

37. The method of claim 1, i) wherein the multispecific antigen binding molecule is a bispecific antibody or bispecific antibody fragment, such as a bispecific T-engaging antibody (BiTE), a dual-affinity re-targeting molecule (DART), or a tandem diabody (TandAb), or ii) wherein the multispecific antigen binding molecule is a bispecific antibody selected from a bispecific antibody listed in Table 3.

38. (canceled)

39. The method of claim 1, wherein the multispecific antigen binding molecule is administered to the subject conjointly with a second multispecific antigen binding molecule with a first antigen binding region specific for a second target antigen and a second antigen binding region specific for a CD3 protein.

40. (canceled)

41. The method of claim 39, wherein the second target antigen is selected from any one of the TAAs listed in Table 2, or
wherein the second target antigen is a CD20 protein, or
wherein the second multispecific antigen binding molecule is selected from any one of the multispecific antigen binding molecules in Table 5.

42-43. (canceled)

44. The method of claim 39, wherein the multispecific antigen binding molecule demonstrates a costimulatory effect when administered conjointly with the second multispecific antigen binding molecule, and wherein the costimulatory effect is one or more of the following: activating T-cells, inducing IL-2 release, inducing CD25+ up-regulation in PBMCs, and increasing T-cell mediated cytotoxicity.

45. (canceled)

46. The method of claim 1, wherein the tumor cells are tumor cells of a B cell cancer.

47. (canceled)

48. The method of claim 1, wherein the tumor is a solid tumor.

49-51. (canceled)

52. The method of claim 1, wherein the method further comprises administering an additional anti-cancer agent.

53. (canceled)

54. The method of claim 52, wherein the additional anti-cancer agent is an immune checkpoint inhibitor, and wherein the immune checkpoint inhibitor is an anti-PD-1 antibody, an anti-PDL1 antibody, an anti-CTLA4 antibody, or an anti-LAG3 antibody.

55-56. (canceled)

57. A method of treating cancer in a subject with a tumor, inducing killing of tumor cells in a tumor in a subject, or inducing T cell activation against tumor cells in a tumor in a subject, the method comprising administering to the subject:
a first multispecific antigen binding molecule with an antigen binding region specific for a first target antigen and an antigen binding region specific for a CD28 protein; and
a second multispecific antigen binding molecule with an antigen binding region specific for a second target antigen and an antigen binding region specific for a CD3 protein,
wherein the first target antigen is not the same antigen as the second target antigen.

58-112. (canceled)


SEQUENCE LISTING (7 sequences)

SEQ ID NO: 1 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 4 - protein, 234 aa, Homo sapiens
MPCGLSALIMCPKGMVAVVVAVDDGDSQALAGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 150 aa, Homo sapiens
MPCGLSALIMCPKGMVAVVVAVDDGDSQALAGNKILVKQSPMLVAYDNAVNLSYNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 136 aa, Homo sapiens
MLRLLLALNLFPSIQVTGNKILVKQSPMLVAYDNAVNLSYNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - RNA, 20 nt, synthetic construct
  source 1..20: mol_type = other RNA
ccggtgcacctcaatgacag